(19) United States
(12) Patent Application Publication
(10) Pub. No.: US 2022/0290132 A1
(43) Pub. Date: Sep. 15, 2022

(54) Engineered CRISPR/Cas9 Systems for Simultaneous Long-term Regulation of Multiple Targets

(71) Applicant: The Penn State Research Foundation, University Park, PA (US)

(72) Inventors: Howard Salis, State College, PA (US); Alexander Reis, Austin, TX (US); Sean Halper, Beltsville, MD (US); Phillip Clauer, State College, PA (US); Grace E. Vezeau, Bellefonte, PA (US); Daniel P. Cetnar, Bellefonte, PA (US); Ayaan Hossain, State College, PA (US)

(21) Appl. No.: 17/633,321

(22) PCT Filed: Aug. 6, 2020
(86) PCT No.: PCT/US20/45145

§ 371 Date: Feb. 7, 2022

Related U.S. Application Data
(60) Provisional application No. 62/883,232, filed on Aug. 6, 2019.

(51) Int. Cl. C12N 15/11; C12N 9/22; C12N 15/90

(52) U.S. Cl. CPC C12N 15/11; C12N 9/22; C12N 15/907; C12N 2800/80; C12N 2310/20; A61K 38/00

(57) ABSTRACT

The invention provides CRISPR-based compositions and methods comprising non-repetitive sgRNA promoter and handle sequences for simultaneous, stable expression of multiple sgRNAs.

### CROSS REFERENCE TO RELATED APPLICATIONS

This application claims priority to U.S. Provisional Application No. 62/883,232, filed Aug. 6, 2019, which is hereby incorporated by reference herein in its entirety.

### STATEMENT REGARDING FEDERALLY SPONSORED RESEARCH OR DEVELOPMENT

This invention was made with government support under Grant No. N00014-13-1-0074 awarded by the United States Navy/ONR and under Hatch Act Project No. PEN04561 awarded by the United States Department of Agriculture/NIFA. The Government has certain rights in the invention.

### BACKGROUND OF THE INVENTION

Engineered CRISPR-based systems have been applied to bind, edit, and cut genomic DNA at specified locations (Dominguez et al., 2016, Nature reviews Molecular cell biology 17, 5; Barrangou et al., 2017, Nature microbiology 2, 17092; Halperin et al., 2018, Nature, 1; Peters et al., 2019, Nature Microbiology 4, 244-250). Many biotechnology applications require editing, modification, or gene regulation at many distinct genomic locations simultaneously. For example, to treat many genetic diseases, it will be necessary to modify nucleotide composition at several locations in a genome, particularly at locations with single nucleotide polymorphisms (Komor et al., 2016, Nature 533, 420; Hess et al., 2017, Molecular cell 68, 26-43). More generally, to reversibly alter a cell's state, it will be necessary to regulate many endogenous genes at the same time (Klann et al., 2018, Current opinion in biotechnology 52, 32-41), or to study complex gene regulatory networks or polygenic diseases (Adamson et al., 2016, Cell 167, 1867-1882. e1821; Swiech et al., 2015, Nature biotechnology 33, 102-106). When using CRISPR-based systems, targeting each distinct location in a genome requires the expression of an additional crRNA or sgRNA (Adamson et al., 2016, Cell 167, 1867-1882. e1821). While multiple guide RNAs have been co-expressed for binding, editing, or gene regulation at multiple locations, these sgRNA arrays have always contained several long DNA repeats within both the guide RNAs and the genetic parts used to express them (Yao et al., 2015, ACS synthetic biology 5, 207-212; Zhao et al., 2018, Biotechnology journal 13, 1800121; Kim et al., 2017, Microbial cell factories 16, 188; Ordon et al., 2017, The Plant Journal 89, 155-168). It is well known that genetic systems with repetitive DNA sequences are more difficult to assemble in vitro (Hughes et al., 2017, Cold Spring Harbor perspectives in biology 9, a023812). Repetitive DNA sequences also trigger homologous recombination, which can spontaneously excise DNA regions between the repetitive sequences, leading genetic instability in vivo. Homologous recombination is particularly active in microbial organisms used in biotechnology and within the viral vectors utilized for mammalian genetic engineering (Stapley et al., 2017, Phil. Trans. R. Soc. B 372, 20160455; Vos et al., 2009, The ISME journal 3, 199). There have been several published studies reporting observations that repetitive DNA sequences within engineered genetic systems have triggered spontaneous deletions that break the genetic system's intended function (Casini et al., 2018, Journal of the American Chemical Society 140, 4302-4316; Najm et al., 2018, Nature biotechnology 36, 179; Jack et al., 2015, ACS synthetic biology 4, 939-943; Brophy et al., 2014, Nature methods 11, 508; Lovett, 2004, Molecular microbiology 52, 1243-1253). Spontaneous deletions are particularly prevalent when the engineered genetic system inhibits the cell's growth rate, therefore creating selective evolutionary pressure.

Thus, there is a need in the art for improved compositions and methods to simultaneously and stably co-express a large number of guide RNAs without introducing repetitive DNA sequences, allowing for broader application of CRISPR technology. The present invention addresses this unmet need.

### SUMMARY OF THE INVENTION

In one embodiment, the invention relates to a nucleic acid molecule comprising an extra long sgRNA array (ELSA) for expression of at least two sgRNA sequences comprising: nucleotide sequences encoding two or more sgRNA sequence, wherein each sgRNA encoding nucleotide sequence is under the control of a sgRNA promoter and operably linked to a sgRNA handle sequence; wherein the ELSA comprises a maximum shared repeat length of 20 nucleotides or less. In one embodiment, the ELSA comprises a maximum shared repeat length 12 nucleotides or less.

In one embodiment, the ELSA comprises nucleotide sequences for expression of at least 5 sgRNAs.

In one embodiment, the ELSA comprises at least two sgRNA promoter sequences of SEQ ID NO:1-64.

In one embodiment, the ELSA comprises at least two sequences of SEQ ID NO:65-118.

In one embodiment, the invention relates to a system comprising at least one ELSA for expression of at least two sgRNA sequences comprising: nucleotide sequences encoding two or more sgRNA sequence, wherein each sgRNA encoding nucleotide sequence is under the control of a sgRNA promoter and operably linked to a sgRNA handle sequence; wherein the ELSA comprises a maximum shared repeat length of 20 nucleotides or less, and a RNA-guided enzyme or a nucleotide sequence encoding a RNA-guided enzyme. In one embodiment, the ELSA comprises a maximum shared repeat length 12 nucleotides or less.

In one embodiment, the ELSA comprises nucleotide sequences for expression of at least 5 sgRNAs.

In one embodiment, the ELSA comprises at least two sgRNA promoter sequences of SEQ ID NO:1-64.

In one embodiment, the ELSA comprises at least two sequences of SEQ ID NO:65-118.

In one embodiment, the nucleotide sequence encoding a RNA-guided enzyme encodes an enzyme selected from the group consisting of a Cas9 enzyme and a catalytically dead Cas9.

In one embodiment, the invention relates to a cell comprising at least one ELSA for expression of at least two sgRNA sequences comprising: nucleotide sequences encoding two or more sgRNA sequence, wherein each sgRNA encoding nucleotide sequence is under the control of a sgRNA promoter and operably linked to a sgRNA handle sequence; wherein the ELSA comprises a maximum shared repeat length of 20 nucleotides or less, and a RNA-guided enzyme or a nucleotide sequence encoding a RNA-guided enzyme. In one embodiment, the ELSA comprises a maximum shared repeat length 12 nucleotides or less.

In one embodiment, the invention relates to a method of modulating the level or activity of one or more target gene comprising contacting a sample with a system comprising at least one ELSA for expression of at least two sgRNA sequences comprising: nucleotide sequences encoding two or more sgRNA sequence, wherein each sgRNA encoding nucleotide sequence is under the control of a sgRNA promoter and operably linked to a sgRNA handle sequence; wherein the ELSA comprises a maximum shared repeat length of 20 nucleotides or less, and a RNA-guided enzyme or a nucleotide sequence encoding a RNA-guided enzyme.

In one embodiment, the one or more target gene are associated with a biological pathway or process. In one embodiment, the biological pathway or process is cellular sugar catabolism, glycolysis, pentose phosphate pathway, pyruvate metabolism, citrate cycle, glyoxylate cycle, propanoate metabolism, butanoate metabolism, inositol phosphate metabolism, amino acid biosynthesis, nucleotide biosynthesis, fatty acid biosynthesis, terpenoid biosynthesis, steroid biosynthesis, glycan biosynthesis, riboflavin biosynthesis, thiamine biosynthesis, biotin biosynthesis, folate biosynthesis, retinol biosynthesis, polyketide biosynthesis, oxidative phosphorylation, methane metabolism, sulfur metabolism, nitrogen metabolism, photosynthesis, nitrogen fixation, carbon dioxide fixation, immune response, or the inflammatory response pathway.

In one embodiment, the one or more target gene are associated with a disease or disorder. In one embodiment, the disease or disorder is obesity, arthritis, cancer, heart disease, diabetes, depression, gastrointestinal disorders, or asthma.

In one embodiment, the invention relates to a method of treating a disease or disorder in a subject in need thereof, comprising administering to the subject a CRISPR/Cas9 system comprising at least one ELSA for expression of at least two sgRNA sequences comprising: nucleotide sequences encoding two or more sgRNA sequence, wherein each sgRNA encoding nucleotide sequence is under the control of a sgRNA promoter and operably linked to a sgRNA handle sequence; wherein the ELSA comprises a maximum shared repeat length of 20 nucleotides or less, and a RNA-guided enzyme or a nucleotide sequence encoding a RNA-guided enzyme, wherein the ELSA comprises nucleotide sequence for expression of two or more sgRNA specific for genes associated with the disease or disorder. In one embodiment, the disease or disorder is obesity, arthritis, cancer, heart disease, diabetes, depression, gastrointestinal disorders, or asthma.

In one embodiment, the invention relates to a nucleic acid molecule encoding an sgRNA, comprising a targeting sequence and an sgRNA handle sequence, wherein the sequence encoding the sgRNA handle comprises a variant of SEQ ID NO:65, comprising at least 80% identity to SEQ ID NO:65. In one embodiment, the sequence encoding the sgRNA handle is SEQ ID NO:66-SEQ ID NO:118.

In one embodiment, the invention relates to an sgRNA encoded by a nucleic acid molecule comprising a targeting sequence and an sgRNA handle sequence, wherein the sequence encoding the sgRNA handle comprises a variant of SEQ ID NO:65, comprising at least 80% identity to SEQ ID NO:65. In one embodiment, the sequence encoding the sgRNA handle is SEQ ID NO:66-SEQ ID NO:118.

In one embodiment, the invention relates to nucleic acid molecule for expression of at least one sgRNA, comprising a promoter sequence of SEQ ID NO:1-64, or a variant or fragment thereof, operably linked to a sequence encoding an sgRNA.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1A through FIG. 1B, depict schematic flow diagrams of the development of ELSAs. FIG. 1A depicts a flow diagram of the computational design algorithm that utilizes toolboxes of highly non-repetitive genetic parts and 23 design rules to build easily synthesized, genetically stable ELSAs. FIG. 1B depicts a flow diagram demonstrating the generation of a toolbox of highly non-repetitive sgRNA handles by combining biophysical constraints, optimization, and machine learning across 3 design-build-test-learn cycles.

FIG. 2A and FIG. 2B depict the development of a toolbox of non-repetitive cr70 promoters. FIG. 2A depicts results from example experiments demonstrating that the promoter-driven protein expression levels (mRFP1) span a ˜100-fold dynamic range observed in E. coli during exponential growth phase in M9 minimal medium. The dark bar is the promoter strength of the commonly used J23100 control promoter from the Anderson Promoter Library. FIG. 2B depicts an evaluation of the maximum number of non-repetitive promoters for a given maximum repeat length.

FIG. 3A through FIG. 3E, depict results from example experiments demonstrating the design and characterization of non-repetitive sgRNA handles. FIG. 3A depicts the sequence design constraints and mutation frequencies for sgRNA handles across three design rounds. FIG. 3B depicts transcriptional knock-downs of mRFP1 protein expression levels using dCas9sp and either highly functional (HF), moderately functional (MF), or non-functional (NF) sgRNA handles. Bars and error bars represent the mean and standard deviation from three biological replicates. FIG. 3C depicts feature weights from linear discriminant analysis quantify nucleotide importance, showing insensitive or sensitive mutated positions. Asterisks indicate significant values.

FIG. 3D depicts the number of non-repetitive sgRNA handles sharing a maximum repeat length L. FIG. 3E depicts the efficiencies of Cas9sp cleavage using selected sgRNA handles in a 15-minute in vitro cleavage assay. Bars and error bars represent the mean and standard deviation from two replicates.

FIG. 4 depicts mutations and activities for 53-screened non-repetitive sgRNA handles. Shaded highlights show LOA-identified nucleotides critical for sgRNA function. Round 1: G53; Round 2: G27, A41, U44; Round 3: A51. Dot-parentheses structure above the WT sequence contains the repeat:anti-repeat duplex (RAR), stem loop 1 (SL1}, and stem loop 2 (SL2). The stem loop 1 structure wasn't included in the constraint in Round 1.

FIG. 5A and FIG. 5B depict in vitro Cas9 cleavage using selected sgRNA handles. FIG. 5A depicts exemplary agarose gel electrophoresis of linearized plasmid DNA when incubated with Cas9 and a complementary sgRNA. 30 nM of each respective sgRNA was incubated with 30 nM of Cas9 for 10 minutes at 25° C. in 1× NEBuffer 3.1. 3 nM of each respective linearized target DNA was then added to the reaction, and incubated for 15 minutes at 37° C. C indicates the no-Cas9 digestion control, WT indicates the wild-type sgRNA handle sequence, and numbered lanes indicate sgRNA handle variants. The uncleaved linear DNA band is 4358 bp, while the two cleaved product bands are 2979 bp and 1379 bp. This assay was repeated twice (N=2). FIG. 5B depicts a comparison of the in vitro (Cas9) and in vivo (dCas9) performances of the selected sgRNA handles.

FIG. 6A through FIG. 6D depict electrophoretic mobility shift assays of sgRNA:Cas9 binary-complex formation. FIG. 6A depicts agarose gel electrophoresis of RNAs, with and without additional Cas9. 30 nM of each RNA was incubated with 0 nM (−) or 30 nM (+) of Cas9 in 1×NEBuffer 3.1 for 10 minutes at 25° C., followed by 15 minutes at 37° C. The free sgRNA band runs between 150 and 50 bp. FIG. 6B depicts gel intensity profiles of sgRNA:Cas9 EMSAs. Gray lines indicate the normalized pixel intensity of each RNA lane with 0 nM added Cas9, and dark lines indicate the normalized pixel intensity of each RNA lane with 30 nM added Cas9. The gray shaded area on each plot represents the location of the sgRNA band. FIG. 6C depicts the percent complex formation of each sgRNA with Cas9, calculated by obtaining the free sgRNA band intensity with and without 30 nM of added Cas9. FIG. 6D depicts the functionality of sgRNAs in vivo and in vitro. Fold-change repression of mRFP1 in an in vivo reporter repression assay is shown as dark bars, and cleavage efficiency of a DNA target in vitro is shown as lighter grey bars.

FIG. 7A through FIG. 7E depict the design, expression, and application of extra-long sgRNA arrays (ELSAs). FIG. 7A depicts the basic expression unit of one sgRNA in a bacterial ELSA. FIG. 7B depicts repeat chord diagrams at L=12 for the natural S. pyogenes CRISPR locus, a 12-sgRNA ELSA using wild-type genetic parts, a 12-sgRNA ELSA using engineered genetic parts, and a 20-sgRNA ELSA using engineered genetic parts. FIG. 7C depicts the part compositions of a 20-sgRNA ELSA targeting 6 genes, called ELSA-Succinate. FIG. 7D depicts the part compositions and sgRNA read depths for a 22-sgRNA ELSA targeting 13 genes, called ELSA-Stress, and a 15-sgRNA ELSA targeting 9 genes, called ELSA-MultiAux. Bars represent the mean read depths from two biological replicates. FIG. 7E depicts RT-qPCR measurements show the relative mRNA levels of targeted genes in SJ_XTL219-RBS1 E. coli cells expressing (darker bars) ELSA-Succinate, ELSA-Stress, or ELSA-MultiAux, or (lighter grey bars) no-ELSA controls. Numeric fold-change ratios are shown. Bars and error bars represent the mean and standard deviation from three biological replicates.

FIG. 8A through FIG. 8C depict ELSA guide locations for targeted operons. Guide locations and targeted operons are shown for ELSA-Succinate (FIG. 8A), ELSA-Stress (FIG. 8B), and ELSA-MultiAux (FIG. 8C). Bars on top of the schematic show non-template (NT) binding guides, bars below the schematic show template (T) binding guides, and grey block arrows illustrate known promoters. Annotated positions are relative to a selected promoter transcription start site, usually the 5′-most promoter.

FIG. 9A through FIG. 9C depicts real-time quantitative PCR of ELSA-targeted genes. Two different inducer conditions were tested: 0.1% and 1% arabinose for ELSA-MultiAux (FIG. 9A), ELSA-Stress (FIG. 9B), and ELSA-Succinate (FIG. 9C) integrated in the E. coli SJ_XTL219 genome (the original strain with an unmodified RBS, RBSO). mRNA levels for the SJ_XTL219 control strain and the labeled ELSA strains are shown.

FIG. 10 depicts the degenerate RBS sequence used to increase dCas9 translation. Translation initiation rates (TIR) predicted by RBS calculator v2.1. RBSO is the original RBS used in the SJ_XTL219 strain. MAGE-oligol shows the degenerate RBS library designed by RBS Library Calculator. The full MAGE oligo used was: 5′-CTCTCTACTGTTTCTCCATACCCGTTTTTTTGGATAGGAGGAGGTM KRGATGGATAAGAAATACTCAATAGGCTTAGCTATCGGCACAAA-3′. RBSs 1-8 are the RBS sequences in the library.

FIG. 11A and FIG. 11B depict metabolite quantitation of ELSA-Succinate using LC-MS. FIG. 11A depicts a volcano plot of significance via Wilson's t-test versus metabolite fold change. Metabolites were detected in extracellular supernatant after 24-hour growth of SHAR1 and SHAR10 in M9+0.4% glycerol+1% arabinose in duplicate. Statistically significant metabolites level changes greater than 2-fold are colored blue. FIG. 11B depicts succinate concentrations (mM) from an exemplary quantitation experiment.

FIG. 12A and FIG. 12B depict auxotrophy testing of ELSA-MultiAux using drop-out media. FIG. 12A depicts triplicate OD₆₀₀ measurements of control strain and ELSA-MultiAux in amino acid drop-out media for the ELSA-targeted amino-acid biosynthesis pathways. Dilutions were performed at 4, 17, and 29 hours. The poor growth of both the control and the ELSA strain on the isoleucine deprived media is likely due to allosteric regulation of the ilv genes by the supplemented leucine and valine, resulting in insufficient internal isoleucine generation in the control strain. Notably, the growth on media deprived of all three associated amino acids does not suffer from this growth defect. FIG. 12B depicts a comparison of knocked down genes versus conferred growth defect on ELSA-MultiAux. Growth rates were calculated from the final plate of the drop out assay, with growth starting at 29 hours of dCas9 induction. The fold change growth rate is calculated as the ratio of ELSA growth rate and the control strains growth rate under each labeled (−AA) amino acid dropout media condition.

FIG. 13A through FIG. 13C depict comparisons of persister cell survival following antibiotic treatment. Survival frequencies of persisters from two strains, the control (SHAR02) and ELSA-Stress (SHAR11), when treated with one of three antibiotics: 100 μg/mL ampicillin (AMP), 5 μg/ml ofloxacin (OFL), or 5 μg/ml cefixime (CEF). FIG. 13A depicts representative petri dishes showing serial dilutions (white numbers indicate dilution) and colonies for 0-hour, and 6-hour antibiotic treated strains. FIG. 13B depicts colony forming units (CFU/ml). Data are the average of three biological replicates. FIG. 13C depicts the percent survival of the control and ELSA strain shows an 11-fold, 7-fold, and 21-fold reduction in viable persisters when ELSA-Stress is introduced and treated with AMP, OFL, and CEF respectively.

FIG. 14 depicts the characterization of individual sgRNAs co-expressed within ELSAs. Knock-down levels were detected from individual sgRNAs co-expressed within each ELSA by transforming a low-copy mRFP1-reporter plasmid (pSC101) into E. coli SJXTL-RBS1 strains with genome-integrated ELSAs (SHAR10-12). Each reporter plasmid uses a different sgRNA binding site, immediately downstream of the promoter, for transcriptional repression of the mRFP1 reporter. The reporter plasmids were also expressed in the control strain, E. coli SJXTL-RBS1 (SHAR02). Fold change values of mRFP1, fluo (−ELSA)/fluo (+ELSA), are reported along the top of the plots. Fluorescence was measured by flow cytometry during mid-exponential growth phase in M9 minimal media supplemented with all amino acids targeted by MultiAux and 1% arabinose. These experiments were performed in biological triplicate (N=3).

FIG. 15 depicts the characterization of individual sgRNAs in ELSA-SuccinateguldesMultiAuxhandles. An additional ELSA, ELSA-Succinate_{guides}MultiAux_{handles}, that combined non-repetitive handle sequences, found within ELSA-MultiAux, with previously verified guide RNA sequences from ELSA-Succinate, while scrambling sgRNA order. The knock-down levels from the individual sgRNAs co-expressed within the ELSA were measured using a mRFP1 reporter plasmid and flow cytometry assay, performed in biological triplicate (N=3). The light bars are the SJXTL-RBS1 control strain (SHAR02) and the dark bars are the strain containing ELSA-Succinate_{guides}MultiAux_{handles} (SHAR13). Fold-change ratios are labeled.

FIG. 16 depicts exemplary experimental results demonstrating the largest observed mRFP1 knockdown for each non-repetitive sgRNA handle across all ELSAs. The fold change values were tabulated for all of the non-repetitive sgRNA handles, as measured by the mRFP1-reporter plasmid and flow cytometry assays, and the maximum fold change observed was computed in mRFP1 knockdown. 19 non-repetitive sgRNA handles knocked down mRFP1 expression by at least 3-fold.

FIG. 17A through FIG. 17F depict exemplary experimental results demonstrating the effects of ELSAs. FIG. 17A depicts that introducing ELSA-Stress or ELSA-MultiAux into the E. coli SJ_XTL219 genome caused 242 or 60 mRNAs to be differentially expressed, respectively, as determined by transcriptome-wide RNA-Seq and a HISAT2-DESeq2 analysis pipeline (N=2 biological replicates). mRNA levels were repressed or activated by statistically significant amounts. FIG. 17B depicts that measured mRNA knock-down levels were compared using RT-qPCR or RNA-Seq data (R2=0.90, 0.98 for ELSA-Stress, ELSA-MultiAux, respectively). FIG. 17C depicts that ELSA-affected genes are counted, categorized by on-target binding, off-target binding, or indirect cascading effects. FIG. 17D depicts the functions of ELSA-affected genes are shown, categorized by their down-regulation or up-regulation. FIG. 17E depicts ELSA-based repression of targeted genes indirectly led to the regulation of other genes, for example, through co-location in operons or by cascading gene regulation. Numbers show the fold-change in mRNA knock-down or mRNA knock-up, compared to a E. coli SJ_XTL219 control. FIG. 17F depicts that ELSA-Stress created widespread changes in quorum sensing and stress response pathways. n.c. no change.

FIG. 18A and FIG. 18B depicts a comparison of RNA-Seq tools for transcriptome analysis. FIG. 18A depicts exemplary experimental results demonstrating that there was strong agreement between mapping and read counting approaches: HISAT2 coupled with featureCounts, and kallisto for all samples (R2 ranges from 0.95-0.97). Condition 1 is M9 minimal media supplemented with Leucine, Condition 2 is M9 minimal media supplemented with all targeted amino acids in ELSA-MultiAux. FIG. 18B depicts exemplary experimental results demonstrating the use of a consensus approach to identify the set of differentially expressed genes (DEGs) agreed upon between four tools: DESeq1, DESeq2, edgeR, and sleuth.

FIG. 19 depicts the characterization of off-target sites for pls81 sgRNA co-expressed in ELSA-Stress. The mRFP1 expression knock-down levels from 18 mutated, off-target sites for the plsB1 guide RNA found in ELSA-Stress were measured to study how mismatches affected guide targeting using non-repetitive sgRNA handles, using the mRFP1 reporter plasmid and flow cytometry assay. sgRNA binding site sequences are shown with off-target mismatches colored red. The light bars are mRFP1 expression levels when reporter plasmids are transformed into the E. coli SJXTL-RBS1 (SHAR02) control strain. Dark bars are mRFP1 reporter expression levels when reporter plasmids are transformed into the ELSA-Stress strain (SHAR11). Experiments were performed in biological triplicate (N=3).

FIG. 20 depicts a table of flagged candidate off-target CRISPRi sites nearby DEGs. 20 unique, candidate, off-target sites were identified that may explain the statistically significant repression (log 2FoldChange<−1.0, or 2-fold) of 15 unique DEGs. A search range between 500 bp upstream of each DEG's start codon and that DEG's stop codon was used which allowed for both canonical (NGG) and non-canonical 1 PAMs, and a maximum allowed hamming distance of 6 and 1 allowed for the distal (1:10) and proximal (11:20) regions of the off-target sequences respectively. 13 and 2 candidate DEGs, and 18 and 2 unique off-target sites were observed for ELSA-Stress and ELSA-MultiAux, respectively. Of the 20 unique off-target sites, 3 have the canonical NGG PAM (balded/underlined). 23 total GuideID/Target-DEG pairings were included, where some of the guides have the same off-target site for multiple DEGs, which in all cases, are operons with overlapping search regions. The table includes the following fields: ELSA—which ELSA was used for the search, GuideID—the identifier of the guide sequence from the ELSA, DEG—the flagged differentially expressed gene, Target—the off-target sequence, with differences between the guide highlighted red, PAM—the 3 bp sequence following the off-target sequence (canonical PAMs are balded and underlined), Location—the location of the 5′ most bp of the off-target 20mer relative to the start codon of the DEG using the coding strand of the CDS as the reference strand, Strand—the strand that the target string is on relative to the coding strand of the DEG's CDS (minus is non-template (NT) targeting), DistHD—hamming distance in the distal region (1:10), ProxHD—hamming distance in the proximal region (11:20), TotalHD—total hamming distance between off-target and guide sequences.

### DETAILED DESCRIPTION

In one aspect the invention provides an engineered CRISPR-Cas system which comprises at least one Extra Long sgRNA Array (ELSA) for simultaneous, stable expression of multiple sgRNAs. In some aspects, the ELSA of the invention comprises multiple non-repetitive sgRNA promoters, handles, terminators and spacers, allowing for simultaneous expression of multiple sgRNAs with minimal silencing due to recombination within the ELSA.

In one embodiment the system is designed to modulate or alter expression of multiple endogenous genes in concert. In some embodiments, system is designed to modulate or alter expression of multiple endogenous genes that are associated with a biological pathway or process. In some embodiments, system is designed to modulate or alter expression of multiple endogenous genes that are associated with a disease or disorder. Therefore, in various embodiments, the invention relates to methods of use of the ELSA CRISPR-based systems of the invention for modulating the level or activity of one or more genes associated with one or more pathway, process, disease or disorder.

In one embodiment, the invention relates to compositions and methods of modulating the level or activity of one or more genes for the treatment or prevention of a disease or disorder. For example, in one embodiment, the invention relates to compositions and methods for stably inhibiting and/or activating the expression or activity of multiple genes simultaneously.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

For purpose of this invention, amplification means any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. In one embodiment, the amplification method is PCR.

“Antisense,” as used herein, refers to a nucleic acid sequence which is complementary to a target sequence, such as, by way of example, complementary to a target miRNA sequence, including, but not limited to, a mature target miRNA sequence, or a sub-sequence thereof. Typically, an antisense sequence is fully complementary to the target sequence across the full length of the antisense nucleic acid sequence.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary).

“Perfectly complementary” means that all the contiguous residues of a nucleic acid sequence will hydrogen bond with the same number of contiguous residues in a second nucleic acid sequence.

“Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “conjugated” refers to covalent attachment of one molecule to a second molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

As used herein, the term “diagnosis” means detecting a disease or disorder or determining the stage or degree of a disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. The diagnostic methods may be used independently, or in combination with other diagnosing and/or staging methods known in the medical art for a particular disease or disorder.

As used herein, the phrase “difference of the level” refers to differences in the quantity of a particular marker, such as a nucleic acid or a protein, in a sample as compared to a control or reference level. For example, the quantity of a particular biomarker may be present at an elevated amount or at a decreased amount in samples of patients with a disease compared to a reference level. In some embodiments, a “difference of a level” may be a difference between the quantity of a particular biomarker present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In some embodiments, a “difference of a level” may be a statistically significant difference between the quantity of a biomarker present in a sample as compared to a control. For example, a difference may be statistically significant if the measured level of the biomarker falls outside of about 1.0 standard deviations, about 1.5 standard deviations, about 2.0 standard deviations, or about 2.5 stand deviations of the mean of any control or reference group.

The term “control or reference standard” describes a material comprising none, or a normal, low, or high level of one of more of the marker (or biomarker) expression products of one or more the markers (or biomarkers) of the invention, such that the control or reference standard may serve as a comparator against which a sample can be compared.

The term “comparator” describes a material comprising none, or a normal, low, or high level of one of more of the marker (or biomarker) expression products of one or more the markers (or biomarkers) of the invention, such that the comparator may serve as a control or reference standard against which a sample can be compared.

As used herein, the term “domain” or “protein domain” refers to a part of a protein sequence that may exist and function independently of the rest of the protein chain.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The terms “dysregulated” and “dysregulation” as used herein describes a decreased (down-regulated) or increased (up-regulated) level of expression of a miRNA present and detected in a sample obtained from subject as compared to the level of expression of that miRNA in a comparator sample, such as a comparator sample obtained from one or more normal, not-at-risk subjects, or from the same subject at a different time point. In some instances, the level of miRNA expression is compared with an average value obtained from more than one not-at-risk individuals. In other instances, the level of miRNA expression is compared with a miRNA level assessed in a sample obtained from one normal, not-at-risk subject.

The terms “determining,” “measuring,” “assessing,” and “assaying” are used interchangeably and include both quantitative and qualitative measurement, and include determining if a characteristic, trait, or feature is present or not. Assessing may be relative or absolute. “Assessing the presence of” includes determining the amount of something present, as well as determining whether it is present or absent.

“Differentially increased expression” or “up regulation” refers to expression levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments there between than a comparator.

“Differentially decreased expression” or “down regulation” refers to expression levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold lower or less, and any and all whole or partial increments there between than a comparator.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “expression” as used herein is defined as the transcription and/or translation of a particular nucleotide sequence.

As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life—eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this invention it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity.”

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors, in general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. In some embodiments, these are capable of hybridizing to the reference sequence under highly stringent conditions. Generally, in order to maximize the hybridization rate, relatively low-stringency hybridization conditions are selected: about 20 to 25° C. lower than the thermal melting point (Tm). The Tm is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15° C. lower than the Tm. In order to require at least about 70%) nucleotide complementarity of hybridized sequences, moderately-stringent washing conditions are selected to be about 15 to 30° C. lower than the Tm. Highly permissive (very low stringency) washing conditions may be as low as 50° C. below the Tm, allowing a high level of mis-matching between hybridized sequences. Those skilled in the art will recognize that other physical and chemical parameters in the hybridization and wash stages can also be altered to affect the outcome of a detectable hybridization signal from a specific level of homology between target and probe sequences. A sequence capable of hybridizing with a given sequence is referred to as the “complement” of the given sequence.

“Inhibitors,” “activators,” and “modulators” of the markers are used to refer to activating, inhibitory, or modulating molecules identified using in vitro and in vivo assays of endometriosis biomarkers. Inhibitors are compounds that, e.g., bind to, partially or totally block activity, decrease, prevent, delay activation, inactivate, desensitize, or down regulate the activity or expression of endometriosis biomarkers. “Activators” are compounds that increase, open, activate, facilitate, enhance activation, sensitize, agonize, or up regulate activity of endometriosis biomarkers, e.g., agonists Inhibitors, activators, or modulators also include genetically modified versions of endometriosis biomarkers, e.g., versions with altered activity, as well as naturally occurring and synthetic ligands, antagonists, agonists, antibodies, peptides, cyclic peptides, nucleic acids, antisense molecules, ribozymes, RNAi, microRNA, and siRNA molecules, small organic molecules and the like. Such assays for inhibitors and activators include, e.g., expressing endometriosis biomarkers in vitro, in cells, or cell extracts, applying putative modulator compounds, and then determining the functional effects on activity, as described elsewhere herein.

As used herein, an “instructional material” includes a publication, a recording, a diagram, or any other medium of expression which can be used to communicate the usefulness of a compound, composition, vector, method or delivery system of the disclosure in the kit for effecting alleviation of the various diseases or disorders recited herein. Optionally, or alternately, the instructional material can describe one or more methods of alleviating the diseases or disorders in a cell or a tissue of a mammal. The instructional material of the kit of the disclosure can, for example, be affixed to a container which contains the identified compound, composition, vector, or delivery system of the disclosure or be shipped together with a container which contains the identified compound, composition, vector, or delivery system. Alternatively, the instructional material can be shipped separately from the container with the intention that the instructional material and the compound be used cooperatively by the recipient.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

As used herein, “microRNA” or “miRNA” describes small non-coding RNA molecules, generally about 15 to about 50 nucleotides in length, preferably 17-23 nucleotides, which can play a role in regulating gene expression through, for example, a process termed RNA interference (RNAi). RNAi describes a phenomenon whereby the presence of an RNA sequence that is complementary or antisense to a sequence in a target gene messenger RNA (mRNA) results in inhibition of expression of the target gene. miRNAs are processed from hairpin precursors of about 70 or more nucleotides (pre-miRNA) which are derived from primary transcripts (pri-miRNA) through sequential cleavage by RNAse III enzymes. miRBase is a comprehensive microRNA database located at www.mirbase.org, incorporated by reference herein in its entirety for all purposes.

A “mutation,” as used herein, refers to a change in nucleic acid or polypeptide sequence relative to a reference sequence (which is preferably a naturally-occurring normal or “wild-type” sequence), and includes translocations, deletions, insertions, and substitutions/point mutations. A “mutant,” as used herein, refers to either a nucleic acid or protein comprising a mutation.

“Naturally occurring” as used herein describes a composition that can be found in nature as distinct from being artificially produced. For example, a nucleotide sequence present in an organism, which can be isolated from a source in nature and which has not been intentionally modified by a person, is naturally occurring.

The terms “isolated”, “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), single guide RNA (sgRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component.

The term “regulatory element” is intended to include promoters, enhancers, internal ribosomal entry sites (IRES), and other expression control elements (e.g. transcription termination signals, such as polyadenylation signals and poly-U sequences) as well as enhancer elements (e.g., WPRE; CMV enhancers; and the SV40 enhancer.) Regulatory elements include those that direct constitutive expression of a nucleotide sequence in many types of host cell and those that direct expression of the nucleotide sequence only in certain host cells (e.g., tissue-specific regulator sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific.

The terms “underexpress,” “underexpression,” “underexpressed,” or “down-regulated” interchangeably refer to a protein or nucleic acid that is transcribed or translated at a detectably lower level in a biological sample from a woman with endometriosis, in comparison to a biological sample from a woman without endometriosis. The term includes underexpression due to transcription, post transcriptional processing, translation, post-translational processing, cellular localization (e.g., organelle, cytoplasm, nucleus, cell surface), and RNA and protein stability, as compared to a control. Underexpression can be detected using conventional techniques for detecting mRNA (i.e., Q-PCR, RT-PCR, PCR, hybridization) or proteins (i.e., ELISA, immunohistochemical techniques). Underexpression can be 10%, 20%, 30%, 40%, 50%, 60%, 70%, 80%, 90% or less in comparison to a control. In certain instances, underexpression is 1-, 2-, 3-, 4-, 5-, 6-, 7-, 8-, 9-, 10-fold or more lower levels of transcription or translation in comparison to a control.

The terms “overexpress,” “overexpression,” “overexpressed,” or “up-regulated” interchangeably refer to a protein or nucleic acid (RNA) that is transcribed or translated at a detectably greater level, usually in a biological sample from a woman with endometriosis, in comparison to a biological sample from a woman without endometriosis. The term includes overexpression due to transcription, post transcriptional processing, translation, post-translational processing, cellular localization (e.g., organelle, cytoplasm, nucleus, cell surface), and RNA and protein stability, as compared to a cell from a woman without endometriosis. Overexpression can be detected using conventional techniques for detecting mRNA (i.e., Q-PCR, RT-PCR, PCR, hybridization) or proteins (i.e., ELISA, immunohistochemical techniques). Overexpression can be 10%, 20%, 30%, 40%, 50%, 60%, 70%, 80%, 90% or more in comparison to a cell from a woman without endometriosis. In certain instances, overexpression is 1-, 2-, 3-, 4-, 5-, 6-, 7-, 8-, 9-, 10-fold, or more higher levels of transcription or translation in comparison to a cell from a woman without endometriosis.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the terms “treat,” “ameliorate,” “treatment,” and “treating” are used interchangeably. These terms refer to an approach for obtaining beneficial or desired results including, but are not limited to, therapeutic benefit and/or a prophylactic benefit. Therapeutic benefit means eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, treatment may be administered to a patient at risk of developing a particular disease, or to a patient reporting one or more of the physiological symptoms of a disease, even though a diagnosis of this disease may not have been made.

As used herein the term “wild type” is a term of the art understood by skilled persons and means the typical form of an organism, strain, gene or characteristic as it occurs in nature as distinguished from mutant or variant forms. A “wild type” can be a base line. As used herein the term “variant” should be taken to mean the exhibition of qualities that have a pattern that deviates from what occurs in nature.

The term “or” as used herein and throughout the disclosure, generally means “and/or” unless the context dictates otherwise.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The invention is based partly on the generation of an Extra Long sgRNA Array (ELSA) CRISPR based system which can be used to stably co-express 20+ single-guide RNAs for diverse CRISPR applications. In one embodiment, the ELSA system can serve to modulate (i.e., activate or inhibit) expression of multiple target genes. Therefore, in various embodiments, the invention relates to compositions and methods for simultaneous modulating gene expression of multiple targets.

In one embodiment, the present invention is directed to methods and compositions for treatment, inhibition, prevention, or reduction of a disease or disorder using the ELSA CRISPR based system of the invention to modulate the expression of multiple target genes associated with the disease or disorder.

sgRNAs

Generally, an sgRNA is made up of two parts: a crispr RNA (crRNA), a 17-20 nucleotide sequence complementary to the target DNA, and a tracr RNA, (herein referred to as an sgRNA handle), which serves as a binding scaffold for the Cas nuclease The invention is based, in part, on the design of variant sgRNA handles that serve to bind to an RNA guided enzyme (e.g., a Cas nuclease or catalytically dead Cas nuclease), and recruit the RNA guided enzyme to a target DNA sequence. Therefore, in one embodiment, the invention relates to an sgRNA comprising at least one variant sgRNA handle.

The standard or reference sgRNA handle sequence is an RNA encoded by the sequence as set forth in SEQ ID NO:65. The invention provides variants of SEQ ID NO:65. In one embodiment, a variant of SEQ ID NO:65 comprise sequences having at least 80%, 85%, 90%, 95%, 96%, 97%, 98%, or 99% identity to SEQ ID NO:65 and further retains the function of binding to an RNA guided enzyme or homolog or ortholog thereof. Exemplary variant sgRNA handle sequences include, but are not limited to, RNA sequences encoded by SEQ ID NO:66-118. In one embodiment, the invention relates to an sgRNA comprising an RNA sequence encoded by SEQ ID NO:66-118.

The sgRNA of the invention can comprise a spacer sequence. In some embodiments, a spacer extension sequence can modify the expression of an sgRNA by reducing superhelical DNA density in the surrounding DNA regions. In some embodiments, spacer sequences are designed so that they do not bind RNA polymerase, which is the enzyme responsible for transcription. In some embodiments, spacer sequences are designed so that they do not contain the recognition sequences for restriction endonucleases. In some embodiments, spacer sequences are designed so that their nucleotide composition is greater than 30%, 35%, or 40% G or C. In some embodiments, spacer sequences are designed so that their nucleotide composition is less than 60%, 65%, or 70% G or C. In some embodiments, multiple spacer sequences are designed together so that they collectively do not share any repetitive DNA sequences above a maximum shared repeat length. In some embodiments, the maximum shared repeat length may be less than 25, 24, 23, 22, 21, 20, 19, 18, 17, 16, 15, 14, 13, 12, 11, 10, 9, 8, or less than 7 consecutive nucleotides. The spacer sequence can have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, or 7000 or more nucleotides. The spacer sequence can be less than 10 nucleotides in length. The spacer sequence can be between 10-30 nucleotides in length. The spacer sequence can be between 30-70 nucleotides in length.

The sgRNA of the invention can comprise a transcriptional terminator sequence. The transcriptional terminator sequence has chemical properties that cause RNA polymerase to dissociate from the DNA during transcriptional elongation, including a rapidly folding RNA hairpin and a RNA sequence region containing more than 50% A or U by composition. In some embodiments, the transcriptional terminator sequence has similarity to a transcriptional terminator found in the genomes of natural organisms. In some embodiments, the transcriptional terminator sequence is non-natural and was designed to possess a RNA hairpin and a sequence region containing more than 50% A or U by composition. In some embodiments, multiple transcriptional terminator sequences are designed or selected so that they collectively do not share any repetitive DNA sequences above a maximum shared repeat length. In some embodiments, the maximum shared repeat length may be less than 25, 24, 23, 22, 21, 20, 19, 18, 17, 16, 15, 14, 13, 12, 11, 10, 9, 8, or less than 7 consecutive nucleotides.

The sgRNA sequence can comprise one or more moiety that can decrease or increase the stability of a nucleic acid targeting molecule (e.g., a stability control sequence, an endoribonuclease binding sequence, a ribozyme). In one embodiment, the moiety can be a transcriptional terminator sequence. The moiety can function in a eukaryotic cell. The moiety can function in a prokaryotic cell. The moiety can function in both eukaryotic and prokaryotic cells. Non-limiting examples of suitable moieties include: a 3′ poly-adenylated tail, a sequence that forms a dsRNA duplex (i.e., a hairpin), a 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like).

sgRNA Promoters

The invention is based, in part, on the development of promoter sequences for expression of sgRNAs of the invention. Therefore, in one embodiment, the invention relates to nucleic acid molecules comprising a sequence encoding an sgRNA under the control of an sgRNA promoter of the invention. sgRNA promoters of the invention include, but are not limited to, promoter sequences as set forth in SEQ ID NO:1-64, or fragments or variants thereof. Therefore, in one embodiment, the nucleic acid molecules of the invention comprise at least one sgRNA promoter sequences selected from SEQ ID NO:1-64, or fragments or variants thereof. Fragments of the sgRNA promoter sequences may comprises at least 80%, 85%, 90%, 95%, 96%, 97%, 98%, or 99% of the full length sequence as set forth in SEQ ID NO:1-64. Variants of the sgRNA handle sequences may comprise sequences having at least 80%, 85%, 90%, 95%, 96%, 97%, 98%, or 99% identity to the sequences as set forth in SEQ ID NO:1-64, so long as the sequence retains the function of promoting expression of an encoded sgRNA.

### ELSA CRISPR Based System

In one embodiment, the ELSA CRISPR based system of the invention comprises an extra long sgRNA array (ELSA) which contains non-repetitive sgRNA handles and promoters for expression of multiple sgRNAs. This system allows for simultaneous expression of multiple sgRNAs for simultaneous regulation of multiple target nucleic acid molecules.

In various embodiments, the ELSA CRISPR based system of the invention allows for stable, simultaneous modulation of the expression level or activity of one or more gene of interest. Therefore, the present invention includes compositions and methods for modulating the level or activity of a gene or gene product in a subject, a cell, a tissue, or an organ in need thereof. In various embodiments, the compositions of the invention modulates (i.e., increases or decreases) the amount of polypeptide, the amount of mRNA, or the amount of activity of a gene or gene product, or a combination thereof. It will be understood by one skilled in the art, based upon the disclosure provided herein, that an increase in the level of a gene or gene product encompasses an increase in gene expression, including transcription, translation, or both. Similarly, a decrease in the level of a gene or gene product encompasses a decrease in gene expression, including transcription, translation, or both.

Extra Long sgRNA Arrays

The ELSA construct of the invention comprises a nucleic acid molecule that has been designed to be both functional and highly non-repetitive. The ELSA of the invention comprises sequence encoding two or more sgRNA nucleotide guide sequences, as well as two or more sgRNA handle sequences, promoters, terminators, and DNA spacers needed to independently transcribe them. The two or more sgRNA handle sequences, promoters, terminators, and DNA spacers included in the ELSA of the invention are non-repetitive such that they serve the function of allowing expression and CRISPR targeting of the encoded sgRNA, but minimize recombination events within the ELSA.

In various embodiments, the ELSA are designed to have a maximum shared repeat length of less than 25, 24, 23, 22, 21, 20, 19, 18, 17, 16, 15, 14, 13, 12, 11, 10, 9, 8, or less than 7 consecutive nucleotides. For example, for an ELSA with a maximum shared repeat length of 20, no nucleotide sequence greater than 20 nucleotides long is repeated throughout the full length of the ELSA sequence.

In one embodiment, the ELSA of the invention comprises two or more promoter sequences, sgRNA sequences, transcriptional terminator sequences, and/or spacer sequences that are selected and placed within a specific order according to one or more design criteria. In one embodiment, the ELSA of the invention comprises a nucleotide composition of between 30% and 70% G or C. In one embodiment, the ELSA of the invention is designed such that the double-stranded DNA melting temperature of each 20-base pair segment of the ELSA is between 45° C. and 65° C. In one embodiment, the ELSA of the invention is designed such that the ELSA nucleotide sequence does not contain more than 10, 11, 12, 13, 14, 15, 16, 17, 18, 19, or 20 occurrences of a repetitive DNA sequence with a length of 10, 11, 12, 13, 14, 15, 16, 17, 18, 19, or 20 nucleotides. In one embodiment, the ELSA of the invention is designed such that the ELSA nucleotide sequence does not contain one or more sequence motifs. Exemplary sequence motifs that may be excluded from an ELSA of the invention include, but are not limited to, a recognition sequence for a restriction endonuclease, and microsatellite sequences, such as sequences with more than 4 consecutive occurrences of the same nucleotide. In one embodiment, the ELSA of the invention is designed such that a combined promoter sequence, sgRNA sequence, transcriptional terminator sequence, and/or spacer sequence does not generate a sequence with more than 50% similarity to a promoter sequence or 50% similarity to a transcriptional terminator sequence.

In one embodiment, the ELSA of the invention comprises sequence encoding between 2 to 100,000 sgRNA sequences. In one embodiment, the ELSA of the invention comprises sequence encoding at least 2, 3, 4, 5, 6, 7, 8, 10, 11, 12, 13, 14, 15, 16, 17, 18, 19, 20, 21, 22, 23, 24, 25, 26, 27, 28, 28, 30, or more than 30 unique sgRNA sequences, wherein each sgRNA sequences is under the control of a non-repetitive promoter and is operably linked to at least one of a non-repetitive sgRNA handle, a non-repetitive terminator and a spacer. Therefore, in one embodiment, the ELSA comprises at least 2, 3, 4, 5, 6, 7, 8, 10, 11, 12, 13, 14, 15, 16, 17, 18, 19, 20, 21, 22, 23, 24, 25, 26, 27, 28, 28, 30, or more than 30 unique non-repetitive sgRNA promoter sequences and at least 2, 3, 4, 5, 6, 7, 8, 10, 11, 12, 13, 14, 15, 16, 17, 18, 19, 20, 21, 22, 23, 24, 25, 26, 27, 28, 28, 30, or more than 30 unique non-repetitive sgRNA handle sequences for expression of multiple sgRNA.

Exemplary non-repetitive sgRNA promoter sequences that can be included in an ELSA of the invention include, but are not limited to, promoter sequences as set forth in SEQ ID NO:1-64, or fragments or variants thereof. Therefore, in one embodiment, the ELSA comprises at least 2, 3, 4, 5, 6, 7, 8, 10, 11, 12, 13, 14, 15, 16, 17, 18, 19, 20, 21, 22, 23, 24, 25, 26, 27, 28, 28, 30, or more than 30 non-repetitive sgRNA promoter sequences selected from SEQ ID NO:1-64, or fragments or variants thereof. Fragments of the sgRNA promoter sequences may comprises at least 80%, 85%, 90%, 95%, 96%, 97%, 98%, or 99% of the full length sequence as set forth in SEQ ID NO:1-64. Variants of the sgRNA handle sequences may comprise sequences having at least 80%, 85%, 90%, 95%, 96%, 97%, 98%, or 99% identity to the sequences as set forth in SEQ ID NO:1-64, so long as the sequence is non-repetitive with other sgRNA promoter sequences included on an ELSA and further retains the function of promoting expression of an sgRNA.

Exemplary non-repetitive sgRNA handle sequences that can be included in an ELSA of the invention include, but are not limited to, handle sequences as set forth in SEQ ID NO:65-118, or fragments or variants thereof. Therefore, in one embodiment, the ELSA comprises at least 2, 3, 4, 5, 6, 7, 8, 10, 11, 12, 13, 14, 15, 16, 17, 18, 19, 20, 21, 22, 23, 24, 25, 26, 27, 28, 28, 30, or more than 30 non-repetitive sgRNA handle sequences selected SEQ ID NO:65-118, or fragments or variants thereof. Fragments of the sgRNA handle sequences may comprises at least 80%, 85%, 90%, 95%, 96%, 97%, 98%, or 99% of the full length sequence as set forth in SEQ ID NO:65-118. Variants of the sgRNA handle sequences may comprise sequences having at least 80%, 85%, 90%, 95%, 96%, 97%, 98%, or 99% identity to the sequences as set forth in SEQ ID NO:65-118, so long as the sequence is non-repetitive with other sgRNA handle sequences included on an ELSA and further retains the function of binding to an RNA guided enzyme or homolog or ortholog thereof.

### Guide Sequences

The systems and sgRNAs of the invention may include any crRNA sequence. The terms crRNA, guide sequence and guide RNA are used interchangeably. In general, a guide sequence is any polynucleotide sequence having sufficient complementarity with a target polynucleotide sequence to hybridize with the target sequence and direct sequence-specific binding of a CRISPR complex to the target sequence, in some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustaiW, Clustal X, BLAT, Novoalign, ELAND (Illumina, San Diego, Calif.), SOAP, and Maq. In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. In some embodiments, the guide sequence is 10-30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, or an assessment of modulation of the level of the target's expression or activity.

Cleavage or modulation of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence may be selected to target any target sequence. In some embodiments, the target sequence is a sequence within a genome of a cell.

In various embodiments, the ELSA of the invention encode multiple sgRNAs which target multiple genes in a pathway or process, or multiple genes associated with a disease or disorder. Exemplary pathways or processes that can be targeted by an ELSA of the invention include, but are not limited to, amino acid, biosynthesis, cellular stress response, and cellular metabolite synthesis or digestion as described below. However, these pathways or processes are not limiting, as any pathway or process involving two or more genes can be targeted for disruption using an ELSA of the invention.

ELSA-Succinate

In one embodiment, the ELSA of the invention encodes two or more sgRNAs specific for one or more genes involved in a metabolite biosynthesis pathway. In one embodiment, the ELSA of the invention comprises a sequence encoding 20 sgRNAs targeting 6 genes in the succinate biosynthesis pathway, ackA, ic1R, poxB, pta, sdhC, sdhD (ELSA-Succinate). In one embodiment, the ELSA-succinate comprises a sequence as set forth in SEQ ID NO:119.

ELSA-MultiAux

In one embodiment, the ELSA of the invention encodes two or more sgRNAs specific for one or more genes involved in the amino acid biosynthesis pathway. In one embodiment, the ELSA of the invention comprises a sequence encoding 15 sgRNAs targeting 9 genes in the amino acid biosynthesis pathway, hisD, proC, lysA, tyrA, aroF, pheA, leuA, ilvD, argH (ELSA-MultiAux). In one embodiment, ELSA-MultiAux comprises two or more nucleic acid molecules that are integrated into the host genome at two or more different locations. In one embodiment, ELSA-MultiAux comprises SEQ ID NO:120 and SEQ ID NO121.

ELSA-Stress

In one embodiment, the ELSA of the invention encodes two or more sgRNAs specific for one or more genes involved in pH homeostasis, quorum sensing, stress response, or essential membrane biosynthesis. In one embodiment, the ELSA of the invention comprises a sequence encoding 22 sgRNAs targeting 13 genes responsible for pH homeostasis, quorum sensing, stress response, and essential membrane biosynthesis, adiA, ansP, dgkA, ic1R, marR, mreC, narQ, plsB, wzb, ycfS, yncE, yncG, and yncH (ELSA-Stress). In one embodiment, ELSA-Stress comprises two or more nucleic acid molecules that are integrated into the host genome at two or more different locations. In one embodiment, ELSA-stress comprises SEQ ID NO:122 and SEQ ID NO:23.

RNA Guided Enzyme

In some embodiments, the RNA-guided enzyme is a Cas9 endonuclease. In some embodiments, the RNA-guided nuclease is a Cpf 1 nuclease. Other RNA-guided nucleases may be used. In some embodiments, the Cas9 endonuclease or Cpf 1 endonuclease is selected from S. pyogenes Cas9, S. aureus Cas9, N. meningitides Cas9, S. thermophilus CRISPR1 Cas9, S. thermophilus CRISPR 3 Cas9, T. denticola Cas9, L. bacterium ND2006 Cpfl and Acidaminococcus sp. BV3L6 Cpfl.

In one embodiment, the system of the invention comprises a Cas9 enzyme or a homolog, an ortholog or mimic thereof. Orthologs of Cas9 may be from a genus which includes but is not limited to Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter. In some embodiments, the Cas9 enzyme, or a homolog, an ortholog or mimic thereof binds to the DNA via the sgRNA, and has cleavage or nickase activity, such that a break or nick is introduced at the target site.

In some embodiments, the Cas9 enzyme comprises catalytically dead Cas9 or a homolog, an ortholog or mimic thereof. Catalytically dead Cas9 mimics include, but are not limited to, proteins or peptides which are capable of interaction with an sgRNA to target a site of interest. Catalytically dead or inactive Cas9, and homologs, orthologs or mimics thereof are referred to herein collectively as “dCas9.”

In some aspects, dCas9 binds to the DNA via the sgRNA, but dCas9 lacks cleavage or nickase activity. In one embodiment dCas9 or ortholog thereof has a diminished nuclease activity of at least 60%, 70%, 80%, 90%, 91%, 92%, 93%, 94%, 95%, 96, 97%, 98%, 99% or 100% as compared with a wild-type Cas9 enzyme or ortholog. In one embodiment, a dCas9 comprises one or more mutations in its catalytic domain which disrupt or inactivate the nuclease activity of the Cas9 enzyme.

Nucleic Acid Molecules

In some embodiments, the composition of the invention comprises an isolated nucleic acid molecule encoding one or more of an sgRNA or ELSA described herein. In one embodiment, the composition comprises a nucleic acid molecule encoding an sgRNA comprising a variant sgRNA handle of the invention. In one embodiment, the composition comprises one or more isolated nucleic acid molecules encoding at least 2, 3, 4, 5, 6, 7, 8, 10, 11, 12, 13, 14, 15, 16, 17, 18, 19, 20, 21, 22, 23, 24, 25, 26, 27, 28, 28, 30, or more than 30 unique sgRNA sequences, wherein each sgRNA sequences is associated with a non-repetitive promoter and sgRNA handle. In one embodiment, the nucleic acid molecule comprises at least 2, 3, 4, 5, 6, 7, 8, 10, 11, 12, 13, 14, 15, 16, 17, 18, 19, 20, 21, 22, 23, 24, 25, 26, 27, 28, 28, 30, or more than 30 unique non-repetitive sgRNA promoter sequences selected from SEQ ID NO:1-64, or fragments or variants thereof. In one embodiment, the nucleic acid molecule comprises at least 2, 3, 4, 5, 6, 7, 8, 10, 11, 12, 13, 14, 15, 16, 17, 18, 19, 20, 21, 22, 23, 24, 25, 26, 27, 28, 28, 30, or more than 30 unique non-repetitive sgRNA handle sequences selected from SEQ ID NO:65-118, or fragments or variants thereof.

Further, the invention encompasses an isolated nucleic acid having substantial sequence identity to a nucleotide sequence disclosed herein. In some embodiments, the isolated nucleic acid molecule comprises one or more sgRNA promoter sequence having at least 80%, 85%, 90%, 95%, 96%, 97%, 98%, or 99% sequence identity with a sgRNA promoter sequence selected from SEQ ID NO:1-64. In some embodiments, the isolated nucleic acid molecule comprises one or more sgRNA handle sequence having at least 80%, 85%, 90%, 95%, 96%, 97%, 98%, or 99% sequence identity with a sgRNA handle sequence selected from SEQ ID NO:65-118.

In one embodiment, the system comprises a combination of nucleic acid molecules, wherein each nucleic acid molecule comprises one or more non-repetitive sgRNA promoter and one or more non-repetitive sgRNA handle for expression of at least one sgRNA. In one embodiment, the system combination of nucleic acid molecules, wherein each nucleic acid molecule comprises wherein each nucleic acid molecule comprises two or more non-repetitive sgRNA promoters and two or more non-repetitive sgRNA handle for expression of at least two sgRNA.

In some aspects the composition of the present invention comprises one or more vectors for expression of one or more ELSA described herein. Vectors allow or facilitate the transfer of an entity from one environment to another. It is a replicon, such as a plasmid, phage, or cosmid, into which another DNA segment may be inserted so as to bring about the replication of the inserted segment. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety.

In some embodiments, a vector comprises one or more regulatory elements. Regulatory elements include those that direct constitutive expression of a nucleotide sequence in many types of host cell and those that direct expression of the nucleotide sequence only in certain host cells (e.g., tissue-specific regulator sequences). In various embodiments, the vector comprises one or more promoters, enhancers, internal ribosomal entry sites (IRES), and other expression control elements (e.g. transcription termination signals, such as polyadenylation signals and poly-U sequences) and enhancer elements (e.g., WPRE; CMV enhancers; and the SV40 enhancer.) Examples of promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EFlα promoter. It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Vectors can be designed for expression of ELSAs in prokaryotic or eukaryotic cells. For example, ELSAs can be expressed in bacterial cells such as Escherichia coli, insect cells (using baculovirus expression vectors), yeast cells, or mammalian cells. Alternatively, the recombinant expression vector can be transcribed and translated in vitro, for example using T7 promoter regulatory sequences and T7 polymerase.

Vectors may be introduced and propagated in a prokaryote or prokaryotic cell, in some embodiments, a prokaryote is used to amplify copies of a vector to be introduced into a eukaryotic cell or as an intermediate vector in the production of a vector to be introduced into a eukaryotic ceil (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pR(T5 (Pharmacia, Piscataway, N.J.) that fuse glutathione S-transferase (GST), maltose E binding protein, or protein A, respectively, to the target recombinant protein. In some embodiments, a vector is a yeast expression vector. Examples of vectors for expression in yeast Saccharomyces cerivisae include pYepSecl (Baldari, et al., 1987. EMBO J. 6: 229-234), pMFa (uijan and Herskowitz, 1982. Cell 30: 933-943), pJRY88 (Schultz et al, 1987. Gene 54: 1 13-123), pYES2 (Invitrogeii Corporation, San Diego, Calif.), and picZ (InVitrogen Corp, San Diego, Calif.). In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 4th ed., Cold Spring Harbor Laboratory, Cold Spring Harbor Laboratory Press, Cold Spring Harbor, N.Y., 2012.

In some embodiments, the recombinant mammalian expression vector is capable of directing expression of the nucleic acid in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Caiame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Grass, 1990. Science 249: 374-379) and the a-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other embodiments of the invention may relate to the use of viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. Tissue specific promoters and/or stage specific promotes may be used to provide temporal and/or spatial control, e.g., by controlling expression of one or more of the sgRNA or the RNA-guided enzyme.

In some embodiments, the composition comprises one or more vectors encoding one or more ELSA CRISPR based system components described herein. For example, in one embodiment, one or more ELSA and a RNA-guided enzyme could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a RNA-guided enzyme and one or more ELSA.

### Generating Non-Repetitive ELSAs

The invention is based, in part, on the development of a method of generating non-repetitive functional sequences for use in an ELSA of the invention. The method of generating non-repetitive functional sequences can be used for generating non-repetitive promoter sequences, sgRNA handles, spacers, or other functional sequences. In one embodiment, a desired function is interaction with a desired protein (e.g., an RNA polymerase or a RNA-guided enzyme.)

In one embodiment, the method comprises generating a pool of variants of a parental sequence, performing RNA structure prediction and Monte Carlo optimization on the pool of variants to identify a subset of variant sequences that satisfy sequence and structural design constraints for retaining a desired function, and eliminating sequences having a shared repeat length greater than a predetermined maximum shared repeat length, thereby generating a pool of non-repetitive functional sequences. In one embodiment, the method includes using a machine learning algorithm to successively improve one or more design constraint across two or more rounds of a design-build-test-learn cycle. Machine learning algorithms that can be used to improve one or more design constraints include, but are not limited to, linear discriminant analysis (LDA), normal discriminant analysis (NDA), discriminant function analysis, Fisher's linear discriminant to identify the mutated nucleotide positions that were associated with breaking sgRNA handle function.

In one embodiment, the invention provides toolboxes of non-repetitive functional sequences generated according to the methods of the invention. A toolbox of non-repetitive functional sequence may comprise at least 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 or more non-repetitive functional sequences, or any number therebetween. In one embodiment, one or more toolbox of non-repetitive functional sequences can be used to generate an ELSA of the invention. For example, in one embodiment, a first toolbox of non-repetitive sgRNA handle sequences and a second toolbox of non-repetitive sgRNA promoter sequences are combined in an algorithm to generate an ELSA sequence.

Therefore, in one embodiment, the invention further comprises one or more software algorithms to process data received from an input source and output an ELSA design. The software algorithms may be executed on an appropriate computing device. Some or all of the software algorithms may be executed on a remote computing device, for example on a server or cloud computing instance connected to the Internet. The software algorithms of the present invention may incorporate machine learning algorithms, big data algorithms, or data modeling algorithms.

In one embodiment, the input source is a user, and the data received is one or more desired target gene or protein. In one embodiment, the software algorithm of the invention (i) identifies one or more target-specific guide sequence to the input target(s), (ii) eliminates candidate guide RNA sequences predicted to have substantial off-target binding activity, (iii) minimizes mis-hybridization events during DNA fragment synthesis via ligation assembly or polymerase cycling assembly; (iv) removes polymeric sequences prone to DNA replication error; (v) minimizes the reduced expression of sgRNAs by premature transcriptional termination or anti-sense RNA expression and (vi) outputs a predicted ELSA nucleotide sequence.

### Methods

In one embodiment, the invention provides a method of regulating the level or activity of multiple target genes simultaneously. For example, in some embodiments, the method is used to modulate the expression of multiple genes associated with a pathway, process, or disease.

In some embodiments, the method comprises introducing to a cell or subject one or more ELSA described herein, or one or more nucleic acid molecules encoding one or more ELSA described herein. For example, in one embodiment, the method comprises administering an ELSA comprising sgRNAs targeting multiple genes associated with a pathway, or process to modulate the pathway, or process. In one embodiment, the method comprises administering an ELSA comprising sgRNAs targeting multiple genes associated with a disease or disorder to treat or prevent the disease or disorder. The method of use of the ELSA is not limited, and therefore the ELSA may be used in any method or process in which modulation of multiple genes is desired, including, but not limited to, gene therapy, CAR T therapy, basic biological research, development of biotechnology products, agricultural applications, and treatment of diseases, among others.

In one embodiment, the invention provides a method of treating a subject for a disease or disorder, comprising modulating gene expression of one or more disease-associated genes by administering to the subject at least one polynucleotide encoding an ELSA of the invention, wherein the ELSA comprises at least two sgRNAs specific for the one or more disease-associated genes. Use of the present system in the manufacture of a medicament for such methods of treatment are also provided.

In some embodiments, one or more vectors driving expression of one or more elements of an ELSA CRISPR system are introduced into a host cell such that expression of the elements of the ELSA CRISPR system direct formation of a CRISPR complex at one or more target sites. Delivery vehicles, vectors, particles, nanoparticles, formulations and components thereof for expression of one or more elements of a CRISPR system are as used in the foregoing documents, such as WO 2014/093622 (PCT/US2013/074667).

One or more ELSA constructs may be used to target CRISPR activity to multiple different, corresponding target sequences within a cell. For example, a single ELSA vector may comprise about or more than about 1, 2, 3, 4, 5, 6, 7, 8, 9, 10, 11, 12, 13, 14, 15, 16, 17, 18, 19, 20, or more guide sequences, wherein each of the guide sequences is under the control of a non-repetitive sgRNA promoter and non-repetitive sgRNA handle.

Two or more encoding components of the ELSA CRISPR-based system of the invention may be delivered separately or together. In one embodiment, a construct encoding a RNA-guided enzyme might be administered at least 1-12 hours prior to the administration of an ELSA construct. Alternatively, a construct encoding a RNA-guided enzyme and an ELSA construct can be administered together. In one embodiment, at least one additional administrations of a construct encoding a RNA-guided enzyme and/or an ELSA construct might be useful to achieve the most efficient levels of gene expression.

In one aspect, the invention provides methods for using one or more elements of a CRISPR system. The CRISPR complex of the invention provides an effective means for modulating expression of one or more genes in a cell. The CRISPR complex of the invention has a wide variety of utility including modifying (e.g., inactivating or activating) a target polynucleotide in a multiplicity of cell types. As such the CRISPR complex of the invention has a broad spectrum of applications in, e.g., gene therapy, drug screening, disease diagnosis, and prognosis.

The method comprises increasing or decreasing expression of a target polynucleotide by using a CRISPR complex that binds to target sequences within, flanking or adjacent to the polynucleotide. In some methods, a target polynucleotide can be inactivated to effect the modification of the expression in a cell. For example, upon the binding of a CRISPR complex to a target sequence in a cell, the target polynucleotide is inactivated such that the sequence is not transcribed, the coded protein is not produced, or the sequence does not function as the wild-type sequence does. For example, a protein or microRNA coding sequence may be inactivated such that the protein or microRNA or pre-microRNA transcript is not produced. In some methods, a control sequence can be inactivated such that it no longer functions as a control sequence. As used herein, “control sequence” refers to any nucleic acid sequence that effects the transcription, translation, or accessibility of a nucleic acid sequence. Examples of a control sequence include, a promoter, a transcription terminator, and an enhancer are control sequences.

In some methods, a target polynucleotide can be activated to effect the modification of the expression in a cell. For example, upon the binding of a CRISPR complex to a target sequence in a cell, the target polynucleotide is activated such that the sequence is transcribed and the coded protein is produced. For example, a protein or microRNA coding sequence may be activated such that the protein or microRNA or pre-microRNA transcript is produced. In one embodiment, a negative regulator of a protein or microRNA coding sequence may be inactivated, and as a consequence the protein or microRNA or pre-microRNA transcript is produced. In some methods, a silent or repressed sequence can be activated such that it is expressed. In some methods, a control sequence can be activated such that it controls the expression of one or more genes or gene products.

The target polynucleotide of a CRISPR complex can be any polynucleotide endogenous or exogenous to the target cell. For example, the target polynucleotide can be a polynucleotide residing in the nucleus of a target cell. In one embodiment, the ELSA CRISPR based system of the invention is designed to target two or more targets within the same cell such that the single ELSA construct modulates multiple targets in a pathway or process simultaneously.

In one embodiment, one or more targeted gene is a disease-associated gene. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

In one embodiment, the compositions and methods of the invention result in increased expression of a gene or gene product relative to the level of a comparator control. In one embodiment, the gene or gene product is increased by at least 1.1 fold, 1.2 fold, 1.3 fold, 1.4 fold, 1.5 fold, 1.6 fold, 1.7 fold, 1.8 fold, 1.9 fold, 2.0 fold, 2.5 fold, 3.0 fold, 3.5 fold, 4.0 fold, 4.5 fold, 5.0 fold, 6.0 fold, 7.0 fold, 8.0 fold, 9.0 fold, 10 fold, 15 fold, 20 fold, 25 fold, 30 fold, 35 fold, 40 fold, 45 fold, 50 fold, or greater than 50 fold relative to a comparator control. In one embodiment, a comparator control is the level of expression of the gene or gene product prior to administration of the ELSA CRISPR-based system of the invention. In one embodiment, a comparator control is a positive control, a negative control, a historical control, a historical norm, or the level of another reference molecule in the biological sample.

In one embodiment, the compositions and methods of the invention result in decreased expression of a gene or gene product relative to the level of a comparator control. In one embodiment, the gene or gene product is decreased by at least 1.1 fold, 1.2 fold, 1.3 fold, 1.4 fold, 1.5 fold, 1.6 fold, 1.7 fold, 1.8 fold, 1.9 fold, 2.0 fold, 2.5 fold, 3.0 fold, 3.5 fold, 4.0 fold, 4.5 fold, 5.0 fold, 6.0 fold, 7.0 fold, 8.0 fold, 9.0 fold, 10 fold, 15 fold, 20 fold, 25 fold, 30 fold, 35 fold, 40 fold, 45 fold, 50 fold, or greater than 50 fold relative to a comparator control. In one embodiment, a comparator control is the level of expression of the gene or gene product prior to administration of the ELSA CRISPR-based system of the invention. In one embodiment, a comparator control is a positive control, a negative control, a historical control, a historical norm, or the level of another reference molecule in the biological sample.

### Genome Editing

The present disclosure provides strategies and techniques for the targeted, specific alteration of the genetic information (genome) of living organisms. As used herein, the term “alteration” or “alteration of genetic information” refers to any change in the genome of a cell. In the context of treating genetic disorders, alterations may include, but are not limited to, insertion, deletion and correction. As used herein, the term “insertion” refers to an addition of one or more nucleotides in a DNA sequence. Insertions can range from small insertions of a few nucleotides to insertions of large segments such as a cDNA or a gene. The term “deletion” refers to a loss or removal of one or more nucleotides in a DNA sequence or a loss or removal of the function of a gene. In some cases, a deletion can include, for example, a loss of a few nucleotides, an exon, an intron, a gene segment, or the entire sequence of a gene. In some cases, deletion of a gene refers to the elimination or reduction of the function or expression of a gene or its gene product. This can result from not only a deletion of sequences within or near the gene, but also other events (e.g., insertion, nonsense mutation) that disrupt the expression of the gene. The term “correction” as used herein, refers to a change of one or more nucleotides of a genome in a cell, whether by insertion, deletion or substitution. Such correction may result in a more favorable genotypic or phenotypic outcome, whether in structure or function, to the genomic site, which was corrected. One non-limiting example of a “correction” includes the correction of a mutant or defective sequence to a wild-type sequence, which restores structure or function to a gene or its gene product(s). Depending on the nature of the mutation, correction may be achieved via various strategies disclosed herein. In one non-limiting example, a missense mutation may be corrected by replacing the region containing the mutation with its wild-type counterpart. As another example, duplication mutations (e.g., repeat expansions) in a gene may be corrected by removing the extra sequences.

In some aspects, alterations may also include a gene knock-in, knock-out or knock-down. As used herein, the term “knock-in” refers to an addition of a DNA sequence, or fragment thereof into a genome. Such DNA sequences to be knocked-in may include an entire gene or genes, may include regulatory sequences associated with a gene or any portion or fragment of the foregoing. For example, a cDNA encoding the wild-type protein may be inserted into the genome of a cell carrying a mutant gene. Knock-in strategies need not replace the defective gene, in whole or in part. In some cases, a knock-in strategy may further involve substitution of an existing sequence with the provided sequence, e.g., substitution of a mutant allele with a wild-type copy. On the other hand, the term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant sequence. Finally, the term “knock-down” as used herein refers to reduction in the expression of a gene or its gene product(s). As a result of a gene knockdown, the protein activity or function may be attenuated or the protein levels may be reduced or eliminated.

Genome editing generally refers to the process of modifying the nucleotide sequence of a genome, preferably in a precise or pre-determined manner. Examples of methods of genome editing described herein include methods of using site-directed nucleases to cut deoxyribonucleic acid (DNA) at precise target locations in the genome, thereby creating single-strand or double-strand DNA breaks at particular locations within the genome. Such breaks can be and regularly are repaired by natural, endogenous cellular processes, such as homology-directed repair (HDR) and non-homologous end joining (NHEJ), as recently reviewed in Cox et al., Nature Medicine 21(2), 121-31 (2015). These two main DNA repair processes consist of a family of alternative pathways. NHEJ directly joins the DNA ends resulting from a double-strand break, sometimes with the loss or addition of nucleotide sequence, which may disrupt or enhance gene expression. HDR utilizes a homologous sequence, or donor sequence, as a template for inserting a defined DNA sequence at the break point. The homologous sequence can be in the endogenous genome, such as a sister chromatid. Alternatively, the donor can be an exogenous nucleic acid, such as a plasmid, a single-strand oligonucleotide, a double-stranded oligonucleotide, a duplex oligonucleotide or a virus, that has regions of high homology with the nuclease-cleaved locus, but which can also contain additional sequence or sequence changes including deletions that can be incorporated into the cleaved target locus. A third repair mechanism can be microhomology-mediated end joining (MMEJ), also referred to as “Alternative NHEJ,” in which the genetic outcome is similar to NHEJ in that small deletions and insertions can occur at the cleavage site. MMEJ can make use of homologous sequences of a few base pairs flanking the DNA break site to drive a more favored DNA end joining repair outcome, and recent reports have further elucidated the molecular mechanism of this process; see, e.g., Cho and Greenberg, Nature 518, 174-76 (2015); Kent et al., Nature Structural and Molecular Biology, Adv. Online doi: 10.1038/nsmb.2961(2015); Mateos-Gomez et al, Nature 518, 254-57 (2015); Ceccaldi et al., Nature 528, 258-62 (2015). In some instances, it may be possible to predict likely repair outcomes based on analysis of potential microhomologies at the site of the DNA break.

Each of these genome editing mechanisms can be used to create desired genomic alterations. A step in the genome editing process can be to create one or two DNA breaks, the latter as double-strand breaks or as two single-stranded breaks, in the target locus as near the site of intended mutation. This can be achieved via the use of site-directed polypeptides, as described and illustrated herein.

### Administration

The ELSA CRISPR-based system, comprising for instance a vector encoding a RNA-guided enzyme, and an ELSA vector for expression of two or more sgRNA, can be delivered using any suitable vector, e.g., plasmid or viral vectors, such as adeno associated vims (AAV), lentivirus, adenovirus or other viral vector types, or combinations thereof. The ELSA CRISPR based system can be packaged into one or more vectors, e.g., plasmid or viral vectors. In some embodiments, the vector, e.g., plasmid or viral vector is delivered to the tissue of interest by, for example, an intramuscular injection, while other times the delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated, the degree of transformation/modification sought, the administration route, the administration mode, the type of transformation/modification sought, etc.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, and/or other compounds known in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

In an embodiment herein the delivery is via an adenovirus, which may be at a single booster dose containing at least 1×10⁵ particles (also referred to as particle units, pu) of adenoviral vector. In an embodiment herein, the dose is at least about 1×10⁶ particles, at least about 1×10⁷ particles, at least about 1×10⁸ particles, at least about 1×10⁹ particles, or at least about 1×10¹⁰ particles of the adenoviral vector.

In an embodiment herein the delivery is via a plasmid. In such plasmid compositions, the dosage should be a sufficient amount of plasmid to elicit a response. The dosage and frequency of administration is within the ambit of the medical or veterinary practitioner (e.g., physician, veterinarian), or scientist skilled in the art. Plasmids of the invention will generally comprise (i) at least two non-repetitive sgRNA promoters; (ii) sequence encoding at least two sgRNAs, operably linked to said promoters; (iii) at least two non-repetitive sgRNA handles, wherein each sgRNA is operably linked to a non-repetitive sgRNA handle sequence; (iv) a selectable marker; (v) an origin of replication; and (vi) a transcription terminator downstream of and operably linked to (ii). The plasmid can also encode the RNA-guided enzyme, but this may instead be encoded on a different vector.

RNA delivery is a useful method of in vivo delivery. It is possible to deliver the ELSA construct into cells using liposomes or nanoparticles. Thus delivery of the ELSA CRISPR system, such as a RNA-guided enzyme and/or and ELSA construct of the invention may be in RNA form and via microvesicles, liposomes or nanoparticles. For example, mRNA encoding an RNA-guided enzyme and one or more ELSA construct can be packaged into liposomal particles for delivery in vivo. Liposomal transfection reagents such as lipofectamine from Life Technologies and other reagents on the market can effectively deliver RNA molecules into cells.

Means of delivery of RNA also include delivery of RNA via nanoparticles (Clio, S., Goldberg, M., Son, S., Xu, Q., Yang, F., Mei, Y., Bogatyrev, S., Langer, R. and Anderson, D., Lipid-like nanoparticles for small interfering RNA delivery to endothelial cells, Advanced Functional Materials, 19: 31 12-3118, 2010) or exosomes (Schroeder, A., Levins, C, Cortez, C, Langer, R., and Anderson, D., Lipid-based nanotherapeutics for siRNA delivery. Journal of Internal Medicine, 267: 9-21, 2010, PMID: 20059641). Indeed, exosomes have been shown to be particularly useful in delivery siRNA, a system with some parallels to the CRISPR system. For instance, Ei-Andaloussi S, et al. (“Exosome-mediated delivery of siRNA in vitro and in vivo.” Nat Protoc. 2012 December; 7(12):2112-26. doi: 10.1038/nprot.2012.131. Epub 2012 Nov. 15) describes how exosomes are promising tools for drag delivery across different biological barriers and can be harnessed for delivery of siRNA in vitro and in vivo. Their approach is to generate targeted exosomes through transfection of an expression vector, comprising an exosomal protein fused with a peptide ligand. The exosomes are then purified and characterized from transfected cell supernatant, then RNA is loaded into the exosomes. Delivery or administration according to the invention can be performed with exosomes.

### Treatment Methods

In one embodiment, the present invention provides methods for treatment, inhibition, prevention, or reduction of a disease or disorder using the ELSA CRISPR-based system of the invention. One of skill in the art, when armed with the disclosure herein, would appreciate that the treating a disease or disorder encompasses administering to a subject an ELSA CRISPR-based system of the invention which comprises sequence encoding at least two sgRNA molecules targeting one or more gene or regulatory region of a gene associated with the disease or disorder to be treated. Additionally, as disclosed elsewhere herein, one skilled in the art would understand, once armed with the teaching provided herein, that the present invention encompasses a method of preventing a wide variety of diseases where increased expression and/or activity of a gene or decreased expression and/or activity of a gene mediates, treats or prevents the disease. Further, the invention encompasses treatment or prevention of such diseases discovered in the future.

For example, in one embodiment, the compositions and methods of the invention are useful for treating or preventing a disease or disorder associated with the immune response, inflammation, or the gut microbiome. Exemplary diseases associated with the stress response include, but are not limited to, obesity, arthritis, cancer, heart disease, diabetes, depression, gastrointestinal disorders, and asthma.

### Pharmaceutical Compositions

The present invention includes pharmaceutical compositions comprising one or more ELSA of the invention. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intratumoral, epidural, intracerebral, intracerebroventricular, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Metabolic Engineering of Organisms

The present invention also pertains to methods for alteration of the metabolism of organisms with the objective of manufacturing chemical compounds. In one embodiment, the present invention provides methods to increase or decrease the expression levels of targeted enzymes inside cells. In one embodiment, the present invention includes methods to target modifications to the expression levels of selected enzymes for the purposeful redirection of carbon, energy, and redox flows inside cells, enabling the accumulation of desired compounds or metabolites. One of skill in the art, when armed with the disclosure herein, would appreciate that altering the expression levels of two or more enzymes can alter a cell's metabolic state so that the cell produces substantially higher or lower amounts of a desired compound or metabolite.

For example, in one embodiment, the compositions and methods of the invention are useful for modifying enzyme expression levels involved in cellular sugar catabolism, glycolysis, pentose phosphate pathway, pyruvate metabolism, citrate cycle, glyoxylate cycle, propanoate metabolism, butanoate metabolism, inositol phosphate metabolism, amino acid biosynthesis, nucleotide biosynthesis, fatty acid biosynthesis, terpenoid biosynthesis, steroid biosynthesis, glycan biosynthesis, riboflavin biosynthesis, thiamine biosynthesis, biotin biosynthesis, folate biosynthesis, retinol biosynthesis, polyketide biosynthesis, oxidative phosphorylation, methane metabolism, sulfur metabolism, nitrogen metabolism, photosynthesis, nitrogen fixation, and carbon dioxide fixation.

For example, in one embodiment, the compositions and methods of the invention are useful to modifying cells to accumulate desired compounds, including, but not limited to, adipic acid, malonic acid, propanol, methylacrylate, acrylic acid, acrylonitrile, ethanolamine, 3-hydroxypropanal, acetol, glycerone, methylglyoxal, glycerate, hyaluronic acid, acetyl acrylic acid, propionic acid, lactic acid, 1,3-butadiene, butanone, 2-butanol, 3-methyl-1-butanol, 2-ketoisocaproate, isovalerate, acetolactate, isobutanol, isobutylene, 2-ketoisovalerate, L-leucine, L-valine, 4-methyl-2-pentanone, terephthalic acid, dihydrobenzenediol, caffeic acid, phenol, dopamine, vanillin, catechol, tyrosol, shikimate, 3-dehydroshikimate, benzaldehyde, phenylethanol, benzyl alcohol, aniline, 4-aminophenylalanine, formic acid, ethanol, farnesol, isopentenol, 1,2-propanediol, hydroxypropionic acid, and succinate.

### Kits

The present invention also pertains to kits useful in the methods of the invention. Such kits comprise various combinations of components useful in any of the methods described elsewhere herein, including for example, compositions comprising at least one non-repetitive sgRNA promoter, handle, or combination thereof for use in the methods of the invention. For example, in one embodiment, the kit comprises components useful for generating an ELSA of the invention. In one embodiment, the kit comprises an ELSA of the invention.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Simultaneous Regulation of Many Genes Using Highly Non-Repetitive Extra-Long sgRNA Arrays

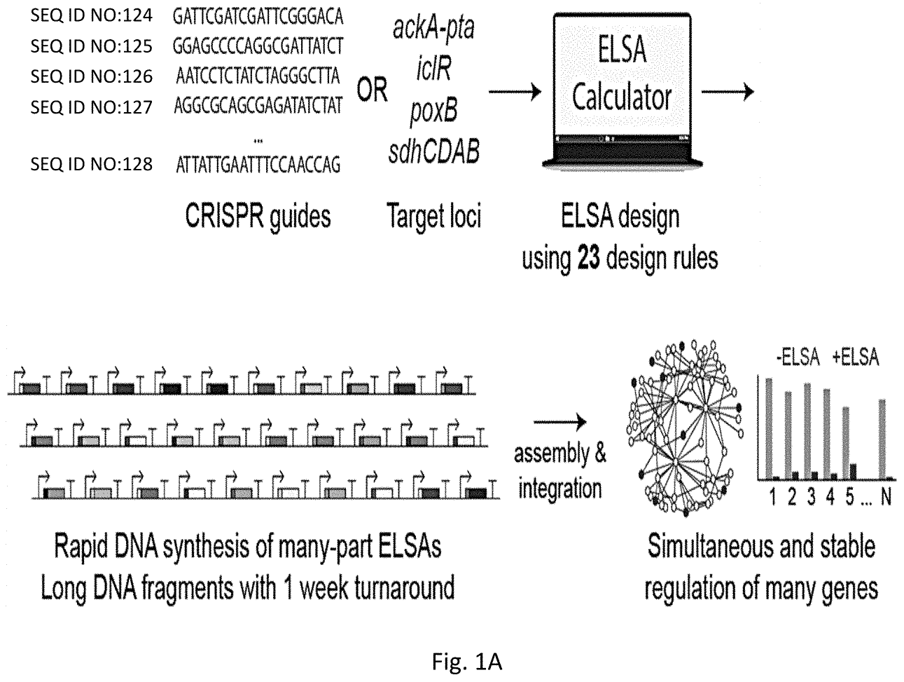

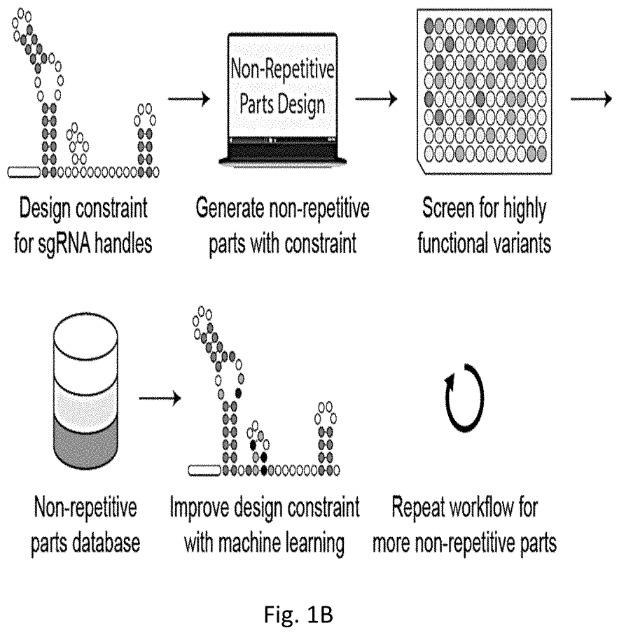

In this work, a scalable approach was developed for co-expression of many single-guide RNAs within extra-long sgRNA arrays (ELSAs), here utilizing deactivated Cas9 from Streptococcus pyogenes to target 22 distinct genomic sites for transcriptional knock-downs. ELSAs are readily synthesized, assembled, integrated into an organism's genome, and expressed to knock down a set of targeted genes simultaneously (FIG. 1A). To do this, the entire DNA sequence must be rationally designed to be both functional and highly non-repetitive, including the single-guide RNAs' 20-nucleotide guide sequences, the sgRNAs' 61-nucleotide handle sequences as well as the promoters, terminators, and DNA spacers needed to independently transcribe them. Toolboxes of highly non-repetitive genetic parts were constructed and combined with an automated design algorithm, to generate ELSA sequences utilizing distinct promoters, sgRNA handles, terminators, and neutral DNA spacers that altogether do not share repetitive DNA sequences (FIG. 1B). Additional design rules are applied to ensure sgRNA expression and minimal off-target sgRNA activity across a selected organism's genome.

Collectively, the experimental results show that ELSAs can be used to regulate many targeted genes simultaneously and to stably introduce highly selective, multi-gene phenotypes without substantial off-target CRISPRi activity. Using the methods of the invention, ELSAs with as many as 100 distinct genetic parts can be designed, synthesized and integrated into the E. coli genome without undesired homologous recombination events. A sequence-structure-function design constraint was also established for Cas9sp sgRNA handles that can now guide the engineering of modified sgRNAs, including activators, switches, and sensors. The design constraint is outstandingly degenerate; more than 10 billion sequences have the necessary nucleotide contacts and overall RNA structure to bind Cas9sp. From that large sequence space, there are more than 100,000 non-repetitive sgRNA handle sequences with a maximum shared repeat of 20 base pairs. These estimates suggest the potential to simultaneously co-express many thousands of sgRNAs in ELSAs without introducing repetitive DNA. The ability to target so many distinct genomic sites would unlock several truly large-scale CRISPR applications, for example, controlling all central metabolic flows from one programmable ELSA, implementing sophisticated genetic circuits with thousands of regulators, and simultaneously editing thousands of SNPs to manipulate cell state.

The materials and methods employed in these experiments are now described.

Characterization of Constitutive Promoters.

Promoters were ordered from IDT as oligonucleotides. Pairs of oligos were annealed and ligated into a BamHI-XbaI-digested flexible test plasmid (pFTV1), replacing the original J23100 promoter, to express a mRFP1 reporter protein. The plasmids were transformed into E. coli K-12 MG1655, and grown in supplemented M9 minimal media over a 16-hour period maintained in the exponential growth phase by multiple serial dilutions. The cells were subsequently sampled, fixed in 1×PBS with 2 mg/mL kanamycin, and their mRFP1 reporter levels measured using flow cytometry. Flow cytometry measurements were performed using a BD LSR Fortessa. 100,000 events were recorded, measurements were filtered to remove non-cell events, geometric means of fluorescence distributions and biological replicates were computed, and cell autofluorescence of untransformed DH10B cells was subtracted to obtain the final reported mean fluorescence values.

Computational Design of Non-Repetitive sgRNA Handles.

First, the original S. pyogenes terminator hairpin was removed from the sgRNA, leaving a 61-nucleotide core handle sequence. For each design round, a custom Python script generated diversified sgRNA handles using Monte Carlo sampling, introducing a selected number of mutations at randomized positions. Mutated sgRNA handle sequences were then compared to the design constraint. If a mutation was located in a conserved base pairing, its complementary nucleotide was also mutated to maintain base pairing. If a mutation was located at an essential nucleotide position, then the mutation was reverted with a high probability. Both the minimum free energy and centroid RNA structures of the mutated sgRNA handles were calculated, and they were accepted only if both RNA structures matched the proposed structural design constraint. Mutated sgRNA handles were then added to the toolbox of non-repetitive sgRNA handles when their maximum shared repeat was L or smaller. To create additional test data, it was allowed that non-repetitive sgRNA handles could mutate at most one essential nucleotide as defined by the design constraint. Lastly, non-repetitive sgRNA handles were matched with terminators from an existing toolbox (Chen et al., 2013, Nature methods 10, 659), ensuring that appending the two sequences did not alter either their minimum free energy or centroid RNA structures and that the resulting toolbox of sgRNA handle-terminator sequences had a maximum shared repeat of L or smaller.

Cloning the sgRNA Handle Test System.

Unless stated otherwise, Escherichia coli K-12 DH10B (Thermo Fisher Scientific) was used for cloning. The non-repetitive sgRNA handles were synthesized as 3-sgRNA arrays on either pUC19 cloning vectors (Genscript) or as gBlock gene fragments (Integrated DNA Technologies or IDT). An existing 3-plasmid test system including pAN-PBAD-sgRNA-A2T (ColE1), pAN-PTet-dCas9 (p15A), and pAN-PA2-RFP (pSC101) was provided by the Voigt lab (Addgene). The sgRNA-expressing plasmids (ColE1) were assembled using ligase cycling reaction (LCR) (Kok et al., 2014, ACS synthetic biology 3, 97-106). Briefly, the sgRNAs and plasmid backbone were PCR amplified with Phusion DNA polymerase (NEB), 5′ phosphates were added via T4 polynucleotide kinase (NEB), and 60 nucleotide oligos were used to mediate blunt-ended ligation using Taq ligase (NEB), resulting in scarless insertion of the sgRNAs downstream of the Ara-pBAD promoter. The mRFP1-expressing target plasmid (pAN-PA2-RFP) was modified to introduce an EcoRI cut site downstream of the constitutive PA2 promoter. The resulting target plasmid was restriction digested with NheI and EcoRI, and oligonucleotides were annealed and inserted into the backbone using T4 DNA ligase (NEB) for each unique target sequence.

Characterization of the Non-Repetitive sgRNA Handles.

Escherichia coli BW27783 (CGSC 12119)43 was used for characterizing the sgRNA handles to ensure strong induction of the pBAD promoter. The BW27783 cells were chemically co-transformed with pAN-PTet-dCas9, and the modified pAN-PBAD-sgRNA and pAN-PA2-RFP plasmids, and plated on ampicillin, kanamycin, and spectinomycin plates. Picked colonies (N=3) were used to inoculate 700 μL LB cultures, and were grown at 37° C. for 9 hours in a shaker incubator. Subsequently, 5 μL of cells were diluted into 195 μL M9 minimal media with 0.4% glycerol, appropriate antibiotics, 20 mM arabinose (Sigma Aldrich), and 1.25 ng/mL anhydrous tetracycline (aTc) (or no inducers for the uninduced condition) in 96-well microplates. The cells were incubated at 37° C. for 5 hours and the OD600 and mRFP1 fluorescence (Ex. 584 nm, Em. 607 nm) was recorded using a TECAN M1000 Infinite plate reader. At the end of the 5-hour growth, cells were mid-exponential phase, and a second identical dilution was done. The second plate was incubated for 12 hours. At the end of the 12-hour culture period, all cells were mid-exponential phase, 20-40 μL of the cell culture was diluted into 200 μL 1×PBS with 2 mg/mL kanamycin for flow cytometry. Flow cytometry measurements and analysis were performed same as before for promoter characterization.

Linear Discriminant Analysis.

Within each design round, the sgRNA handle sequences tested were converted into binary signal vectors, where a value of 1 at position j indicated the presence of the same nucleotide as the WT sgRNA sequence and a value of 0 indicated a mutation. The induced mRFP1 fluorescence values of each sgRNA handle were used to assign each handle into one of two classes, where sgRNA handles with an induced RFP fluorescence of less than or equal to 100 fluo were labeled ‘functional’, or were otherwise ‘non-functional’. Linear discriminant analysis (LDA) was used to infer relative importance (weights) of not changing the nucleotide at each of the positions in the sequence (features). Using LDA with an automatically inferred shrinkage parameter (via Ledoit-Wolf lemma; Ledoit et al., 2004, Journal of multivariate analysis 88, 365-411) helped us select features that were most informative in the classification task for each of the rounds, making the models statistically robust, while eliminating the need for hyper-parameter optimization. For each of the rounds, 10,000 different instances of the LDA model were trained on a random subset of 80% of the binary signal matrix and tested on the entire signal dataset. The instances were optimized using eigenvalue decomposition, and all models with the highest F1 score were extracted as an ensemble. The arithmetic mean of feature weights learned by the models in the ensemble was taken as the predicted importance of not changing nucleotides at different positions and filtered them with the median absolute deviation (MAD) test with a cut-off of 3 to retain the most statistically important features.

In Vitro Cas9 Cleavage Assay.

Linear amplicons were constructed, consisting of the T7 promoter, 2 guanosine residues to promote efficient transcription initiation by T7 RNA polymerase (5′-AAGCTAATACGACTCACTATAGG-3′, transcription start site underlined), and the sgRNA guide and handle. The sgRNAs were transcribed using a HiScribe™ T7 High Yield RNA Synthesis Kit (NEB), and purified via phenol:chloroform extraction followed by ethanol precipitation. Gel electrophoresis was used to confirm the integrity of each sgRNA transcript. Each sgRNA was resuspended to 300 nM in 1×TE buffer, and annealed to renature the RNA by heating to 95° C. for 5 minutes and cooling at 0.2° C. increments per minute to 25° C. The modified pAN-PA2-RFP vectors used for in vivo characterization were used as the target DNA sequence for the in vitro Cas9 cleavage assay. The plasmid vector was linearized by digesting with NdeI (NEB) for 6 hours. In vitro Cas9 cleavage reactions were performed on this linearized target DNA using purified S. pyogenes Cas9 nuclease (NEB). Equimolar sgRNA and Cas9 (30 nM) were incubated in 1×NEBuffer 3.1 (NEB, 100 mM NaCl, 50 mM Tris-HCl, 10 mM MgCl₂, 100 μg/mL BSA, pH 7.9) in a total volume of 30 μL for 10 minutes at 25° C. to facilitate sgRNA loading. 3 nM of the corresponding linearized target DNA was subsequently added, and each reaction was incubated for 15 minutes at 37° C. After digesting with Cas9, 1 μL of Proteinase K (NEB) was added to each reaction, and incubated for 10 minutes at room temperature. The digestion products of each reaction were visualized by running on a 1×TBE, 1% agarose (SeaKem LE, Lonza), 1× GelStar (Lonza) gel. Digital photographs were taken of the gels using a blue light trans-illuminator with an orange filter, and the intensities of the digested product bands were quantified using GelAnalyzer to determine the degree of digestion. For each of the two cleaved bands, the following formula was used to determine the percent cleavage.

$$\% cleavage = \frac{\frac{{I}_{n}}{{len}_{n}}}{\frac{{I}_{n}}{{len}_{n}} + \frac{{I}_{N D}}{{len}_{N D}}}$$

Iₙ is the intensity of a given product band, I_ND is the intensity of the uncleaved plasmid band, and lenₙ and len_{ND} are the lengths of the given product band (2979 or 1379 bp) and the uncleaved plasmid band (4358 bp), respectively. The cleavage efficiency of each Cas9 cleavage reaction was reported as the average of the cleavage efficiencies, determined using both product bands, across two independent replicates.

Electrophoretic Mobility Shift Assay.

Electrophoretic mobility shift assays (EMSAs) were performed to measure the equilibrium formation of sgRNA:Cas9 binary complex (RNP). sgRNAs were produced using in vitro transcription. Briefly, linear DNA templates were constructed combining a T7 promoter, guide RNA sequence, and a selected non-repetitive sgRNA handle. sgRNAs were transcribed using the HiScribe™ T7 High Yield RNA Synthesis Kit (NEB), and purified using phenol:chloroform extraction and ethanol precipitation. Following synthesis and confirmation of transcript integrity via agarose gel electrophoresis, sgRNAs were re-folded at a concentration of 300 nM in 1×TE buffer. Binding assays were performed with 30 nM sgRNA, with or without 30 nM Cas9, in 1× NEBuffer 3.1 buffer (NEB, 100 mM NaCl, 50 mM Tris-HCl, 10 mM MgCl¬2, 100 μg/mL BSA, pH 7.9) in a total volume of 30 μL. Reactions were incubated at 25° C. for 10 minutes, followed by 37° C. for 15 minutes. sgRNA bands were visualized, with and without added Cas9, by running each reaction on a gel containing 1×TAE, 1% agarose (SeaKem LE, Lonza), and 1× GelStar fluorescent dye (Lonza). Digital photographs were taken of gels using a blue light trans-illuminator with an orange filter. Fluorescent band intensities were quantified using GelAnalyzer to determine the amount of unbound sgRNA. The percent complex formation was calculated as the following:

$$\% Cas 9 : sgRNA complex = 1 - \frac{{I}_{A}}{{I}_{B}}$$

I_{A} is the intensity of the free sgRNA band when incubated with 30 nM of Cas9. I_{B} is the intensity of the free sgRNA band when no Cas9 is present during incubation.

Software for ELSA Design.

A software implementation of the design algorithm, called the ELSA Calculator, is available at salislab.net/software. Python source code and a Dockerfile are available at github.com/hsalis/SalisLabCode. The algorithm uses a genetic algorithm to determine the optimal selection and configuration of non-repetitive parts to maximize the probability of synthesis success and genetic stability. Synthesis success is determined by assessing the ELSA sequences for features that inhibit DNA fragment synthesis. These features include repeats, highly structured DNA regions, highly variable GC content regions, highly variable melting temperature regions, and DNA sequence runs (e.g. poly-N). Guides for the ELSAs were preferentially selected to target the non-template strand within or immediately downstream of each promoter expressing the targeted gene.

Construction of the ELSA Strains.

ELSAs were cloned via Gibson assembly into one of two in-house integration vectors containing a resistance marker (KanR or CmR) and 500 bp homology arms to either the intergenic region between galM and gpmA (ACR_IV1) or the intergenic region between yciL and tonB in the E. coli genome. ELSAs were integrated into SJ_XTL219 cells38 via phage λRed recombination. Briefly, SJ_XTL219 cells were transformed with the pORTMAGE-2 plasmid36 (Addgene plasmid #72677), grown overnight at 30° C., and then diluted and grown to an OD of 0.4-0.6. The cells were then heat shocked at 42° C. for 15 minutes, then put on ice for 10 minutes. Cells were centrifuged, washed twice and resuspended with sterile ultrapure water. 50-100 ng of linearized ELSA DNA with flanking homology arms was added to 25 μL of resuspended cells. After one minute of incubation, the cells were electroporated at 1800V and added to 1 mL SOC media for 1-2 hours recovery. 200 μL of recovering cells were plated on selective media containing 25 μg/mL kanamycin (ACR_IV1) or 15 μg/mL chloramphenicol (ACR_IV2). Integration was confirmed by colony PCR. Successfully integrate strains were cured of pORTMAGE-2 by growing the cells on non-selective media at 37° C. for 24-48 hours.

RT-qPCR of Targeted Genes.

All ELSA-containing strains were grown with 25 μg/mL kanamycin and SJ_XTL219 was used as a control throughout. Strains were initially grown to stationary phase for 9 hours in LB, followed by serial dilution into cultures grown using M9 minimal media with 0.5 mM leucine. To measure mRNA levels, strains containing ELSA-MultiAux were grown using M9 minimal media with all targeted amino acids at 0.5 mM (arginine, histidine, isoleucine, leucine, lysine, phenylalanine, proline, tryptophan, and tyrosine). Strains were grown for approximately 15 hours with serial dilution to maintain them in exponential growth phase. After reaching an OD of 0.2, total RNA was extracted using Total RNA Purification Kit (Norgen Biotek Corp.) and DNA was removed using TURBO DNA-free™ Kit (Thermo Fisher Scientific). RNA integrity was confirmed by agarose gel electrophoresis. cDNA of the total RNA samples was produced using High-Capacity cDNA Reverse Transcription Kit (Thermo Fisher Scientific). SYBR Green Real-Time PCR Master Mix (Thermo Fisher Scientific) with custom primers was used for real-time quantitative PCR for all targeted genes on a StepOnePlus™ Real-Time PCR System (Applied Biosystems™). All genes and samples were quantified in biological triplicate unless otherwise stated. A custom python script was used to calculate relative mRNA levels and fold-change knockdown of the ELSA strains relative to the control strain using the AACT method.

Characterization of Individual sgRNAs and plsB1 Off-Targets Using Reporter Plasmids.

The mRFP1 reporter plasmid pAN-PA2-RFP was modified by introducing desired sgRNA binding sites in between NheI and EcoRI restriction sites using annealing of oligonucleotides, digestion, and ligation. Separate reporter plasmids were transformed into the control strain, SHAR02 (SJ_XTL219 galM<KanR MCS> gmpA RBS1-dCas9) and corresponding ELSA strains. All cells were grown in 96-well microtiter plates using M9 minimal media, 1% arabinose and all amino acids, incubated at 37° C. shaking for 11 to 12 hours, serially diluted at least once to maintain cultures in the exponential growth phase, and harvested. Single-cell mRFP1 fluorescence levels were recorded using flow cytometry as before. All characterization was performed in biological triplicate (N=3).

Multiplex Automated Genome Editing (MAGE) to Increase dCas9 Expression.

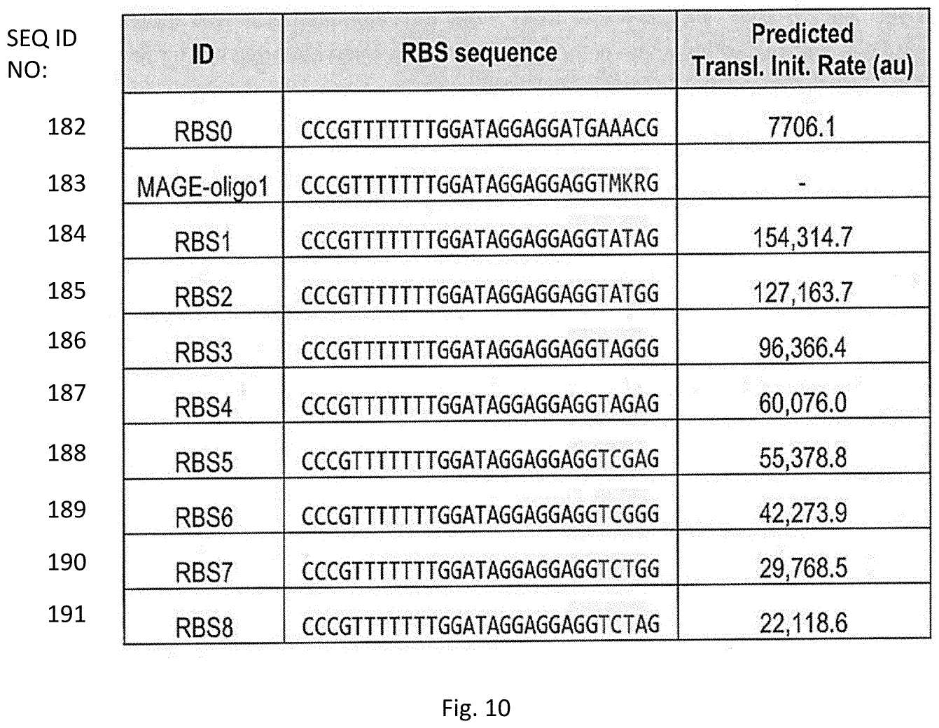

10 cycles of MAGE (Wang et al., 2009, Nature 460, 894) was used to enrich the RBS sequence with a MAGE oligo containing a degenerate RBS sequence (FIG. 10). Unless stated otherwise, all steps used 50 μg/mL ampicillin for selection of the pORTMAGE plasmid. ELSA-containing and control SJ_XTL219 strains were transformed with pORTMAGE2 and grown overnight in a 30° C. shaker in LB. Cells were then diluted 100-fold in 5 mL SOC and grown for 2-4 hours, until the OD reached 0.5-0.7. Cells were then induced via 42° C. water bath for 15 minutes and chilled on ice for 10 minutes. Next, 1 mL of cells were transferred to chilled microcentrifuge tubes and spun in a chilled centrifuge for 30 seconds. The supernatant was removed and the cells were resuspended in 1 mL chilled sterile water and spun down three times. After the final spin, cells were resuspended in 50 uL 2 uM degenerate RBS oligo and transferred to electroporation cuvettes on ice. Cells were electroporated at 1700V, immediately resuspended in 1 mL SOC, and transferred to a culture tube. After 1-1.5 hours of recovery in a 30° C. shaker, the cells were diluted with 4 mL of SOC, beginning a new round of growth for the next cycle. Overnight cultures were saved as glycerol stocks after each day. This cycle was repeated 10 times. The product of the tenth cycle was plated on 25 μg/mL kanamycin plates and colonies were picked for colony PCRs and sequencing. Successfully modified RBS variants were cured of pORTMAGE as described previously.

Auxotrophy Assay

Growth curves were measured of ELSA-MultiAux in supplemented M9 minimal media with either full amino acid supplementation, no amino acid supplementation, or single amino acid drop-outs. Picked colonies of ELSA-MultiAux (N=3) were used to inoculate 700 μL LB cultures and were grown at 37° C. for 9 hours. 5 μL of cells were added into 195 μL M9 minimal media with 0.4% glycerol, appropriate antibiotics, 1% w/v arabinose (Sigma Aldrich), and appropriate full, none, or dropout amino acids sets in 96-well microplates. Each dropout media included all, but one of the following amino acids at 0.5 mM: arginine, histidine, isoleucine, leucine, lysine, proline, phenylalanine, tyrosine, and valine. The cells were incubated at 37° C. for a total of 40 hours and maintained in the exponential growth phase by periodic serial dilution. Growth curves were used to calculate specific growth rates.

Genetic Integrity Assay after Adaptation.

ELSA-containing strains were subjected to an extended period of growth, followed by assessment of their genomic integrity by colony PCR and Sanger sequencing. For ELSA-Stress and ELSA-MultiAux, 3 colonies were picked of each and were grown for a total of 48 hours in 5 mL LB media with appropriate antibiotics at 37° C., and maintained in the exponential phase of growth by repeated serial dilutions every 12 hours. The cells were then diluted to an OD of 0.01 in 5 mL M9, 0.4% glycerol, 1% arabinose and appropriate antibiotics and grown for a total of 55 hours, and maintained in the exponential growth phase by repeated serial dilutions about every 16 hours. Colony PCRs were performed on the resulting colonies to amplify the sgRNA arrays. PCR products were Sanger sequenced, reads were aligned to the reference genome, and their integrity was confirmed. A similar procedure was performed for ELSA-Succinate, except the growth adaptation time was extended to 72 hours in LB media and an additional 72 hours in supplemented M9 minimal media.

Metabolite Quantitation.

Metabolic levels were quantified in ELSA-Succinate and control strain SJ_XTL219-RB S1 using LC-MS. Following adaptation, ELSA-Succinate and control cells were grown in 5 mL LB+1% arabinose at 37 C and 300 rpm shaking for 8 hours. Cultures were then centrifuged and the pellets washed with 1 mL PBS. The pellets were centrifuged again, the PBS removed, and then the culture was resuspended in 5 mL M9, 0.4% glycerol, 1% arabinose, and appropriate antibiotic. Cultures were grown for 24 hours in the same media, followed by centrifugation. Filter-sterilized supernatants and a succinate calibration curve were run on a Thermo Scientific UltiMate 3000 HPLC using a Waters XSelect HSS T3 XP column, followed by a Thermo Scientific Exactive Plus MS. The resulting data were processed using Proteowizard 3.0.18294 MSConvert and MS-DIAL v3.20 into calculated peaks and areas that were then mapped to metabolites of interest.

Persister Formation Assay.

The control SJ_XTL219 RBS-1 (SHAR02) and ELSA-Stress (SHAR11) strains were grown in LB to stationary phase and diluted 1:10 in fresh LB containing one of the three antibiotics: 100 μg/mL ampicillin (AMP), 5 μg/mL ofloxacin (OFL), or 5 μg/mL cefixime (CEF). 20 μL of 0-hour and 6-hour treatments were spot-plated across multiple serial dilutions. For the 6-hour treatment, the AMP treated cells were directly diluted and spot plated, and the OFL/CEF treated cells were washed with PBS, diluted and spot plated. Colony counts were taken after 16 hours of incubation. The serial dilutions containing ˜10-100 colonies were used for the counts. Specifically, the 0-hour counts used the 6× serial dilution plates, the 6-hour AMP used the 2× serial dilutions and 1× dilution for the control and ELSA strains respectively, and the 6-hour OFL/CEF both used the 1× dilution for both strains. The number of colony forming units (CFU/mL) was computed and averaged across three biological replicates. Percent survival rate is the relative survival rate, of a given strain, following 6-hours of antibiotic treatment (CFU at 0-hour/CFU at 6-hour following treatment).

RNA-Seq.

Total RNA was extracted from cultured strains as previously described and its integrity confirmed using a Bioanalyzer (Agilent). Ribosomal RNA depletion was carried out using the Ribo-Zero rRNA Removal Kit for Bacteria (Illumina), and using ethanol precipitation instead of column-based purification to retain short RNAs, including sgRNAs. The integrity of rRNA depleted samples was again confirmed using the Bioanalyzer, followed by library preparation performed by the Penn State Genomics Core Facility. RNA-Seq was carried out using the TruSeq Stranded mRNA Kit (Illumina) and an Illumina NextSeq using 75 bp paired-end sequencing, obtaining ˜20 to 40 million raw reads per sample.

Quality Control of Raw Sequencing Reads.

FASTQ files were initially processed using Trimmomatic46 v0.38 to remove adapter sequences, trim low quality beginning and ends of reads, trim reads with low average quality, and filter out short reads. Illumina-specific adapter sequences were trimmed from the reads using the universal Illumina adapter sequences provided by Trimmomatic (TruSeq3-PE.fa), with a maximum of 2 mismatches, an accuracy match score of 30 between the two adapter ligated reads for PE palindrome read alignment, and an accuracy match score of 10 between any adapter sequence with a read. Bases were trimmed from the start of reads if the Phred quality score was below 33, and trimmed from the end of reads if the threshold quality was below 30. Reads were clipped if the average quality dropped below 15 within any 4-nucleotide sliding window. Post-trimmed reads were dropped if shorter than 36 nucleotides. There were ˜20 to 38 million trimmed reads per sample.

Read Filtering and Partitioning.

Following initial read processing, a kmer filtering approach (BBDuk) was used to filter out rRNA, ncRNA and ELSA RNA in unique, consecutive steps. Reference multi-FASTA files were generated for filtering as follows. The rRNA reference included all ‘rRNA’ features from the Escherichia coli K-12 sub. str. MG1655 RefSeq genome (NC_000913.3). The ncRNA reference included ‘ncRNA’, ‘tmRNA’, and ‘tRNA’ features in the RefSeq genome. Separate references were created for ELSA-MultiAux and ELSA-Stress using the contiguous genome-integrated sequences. For each step, all reads that contained a 31-mer match to the corresponding reference, with one mismatch allowed, were filtered. Less than <1% of the quality-controlled reads were flagged and filtered as rRNA, 19-28% of the remaining reads were filtered out as ncRNA, and a subsequent 2-3% of the reads were then filtered out as ELSA RNA. The remaining unfiltered RNA reads, corresponding to the E. coli transcriptome, were earmarked for downstream transcriptome quantification.

ELSA Read Depth Analysis.

Sequencing coverage (or read depth) was used to confirm sgRNA expression and to identify any read-through or anti-sense transcription across the ELSAs. A short-read aligner, BWA-MEM v0.7.17, was used to map the ELSA RNA reads to the ELSA reference sequences, using a minimum seed length of 31 nucleotides, and both the forward (R2 file) and reverse (R1 file) paired-end reads as input. Supplementary alignments (0x800) were removed, and the remaining aligned reads were sorted and indexed (SAMtools v1.9). A custom Python script using pysam, the python-interface to SAMtools, was then used to obtain the read depth at each position across the ELSAs. For each read pair, at least one of the two reads in each read pair was required to have a MAPQ score of 55 or greater. For stranded analysis, the SAM flag for the first read in the mate pair was used to determine if the RNA read was derived from the coding strand (0x20) or from the template (reverse) strand (0x10), and the depth count of the corresponding strand was incremented for the mapped fragment. DNAplotlib, a Python library for visualization of genetic constructs and associated data47, was used to plot the read depth trace aligned with the ELSA SBOL Visual compliant diagram for the first replicate of ELSA-MultiAux and ELSA-Stress.

Transcriptome Quantification.

The reference transcriptome was created by extracting the coding strand sequences of all ‘CDS’ features from the ReqSeq genome (NC_000913.3) and was used for all subsequent alignment. Two independent mapping approaches were used for quantifying transcriptome abundances from the earmarked files generated above in the read filtering and partitioning step. Kallisto v0.44.0 was invoked with the ‘quant’ command with 500 bootstrap samples and by specifying strand specific reads, with the first read as the forward read as before with BWA-MEM. Separately, HISAT2 v2.1.0 was used to align the reads to the reference transcriptome, specifying paired end reads as before. SAMtools view and merge commands were subsequently used to separate the HISAT2-aligned reads that corresponded to the coding strand sequences from those that corresponded to the reverse strand. A read summarization program, featureCounts, then counted the HISAT2 transcript-mapped paired end reads to obtain a final fragment count for all genes.

Differential Expression Analysis.

Three R packages, DESeq1 v1.32.0, DESeq2 v1.20.0, and edgeR v3.22.5, were used to calculate the differential expression of genes from the HISAT2-aligned reads, all using default settings. A fourth R package, sleuth v0.30.0, was used for differential analysis of the kallisto-aligned reads, using default settings. All genes that were identified as significantly differentially expressed (p<0.05) using all four methods were taken as the consensus differentially expressed genes (DEGs). Consensus DEGs with more than a 2-fold change in expression were flagged for analysis.

Deg Classification.

All DEGs were assigned a functional category based on available databases (EcoCYC48, RegulonDB49) and existing literature. If the gene was a negative regulator associated with a particular function, the functional classification of that gene was assigned to the opposite of the gene's differential expression sign (e.g. tqsA was upregulated and is a negative regulator of quorum sensing; therefore, quorum sensing was classified as being downregulated). DEGs targeted by the ELSA were flagged as “ELSA targeted” (on-target). Candidate off-target sgRNA binding sites affecting DEGs were identified by examining the sequences surrounding repressed DEGs (2-fold or higher) from 500 base pairs upstream of the DEG's start codon to the DEG's stop codon. Sequences were labeled as candidate off-target sgRNA binding sites if they contained at most 1 PAM-proximal mismatches or at most 6 PAM-distal mismatches, compared to any co-expressed sgRNAs, for all canonical and non-canonical PAMs (Farasat et al., 2016, PLoS Computational Biology 12, e1004724). DEGs that were not “ELSA targeted” or “off-target” were classified as “indirect.” This analysis was performed for ELSA-MultiAux and ELSA-Stress.

The results of the experiments are now described.

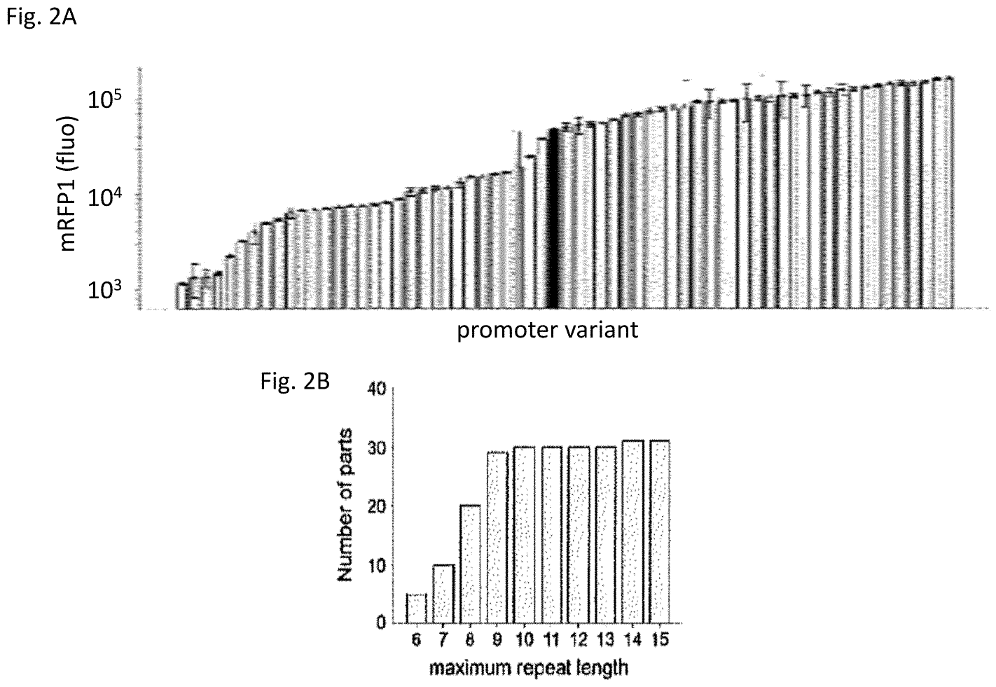

64 constitutive bacterial promoters were designed and constructed that do not share more than 22 base pairs of the same consecutive DNA sequence, called the maximum shared repeat length L. The promoters' transcription rates were characterized using an mRFP1 fluorescent protein reporter assay and flow cytometry. It was found that their transcription rates varied across a 140-fold range (FIG. 2); 33 of these promoters had higher transcription rates than a common reference promoter, J23100. An L of 22 base pairs is sufficient to reduce the rate of homologous recombination to about 1 in 20,000 in rec+E. coli strains (Shen et al., 1986, Genetics 112, 441-457). However, a genetic system must be even more non-repetitive (an L of 12 base pairs) to ensure its successful synthesis and assembly using non-clonal DNA fragments with a 5-day synthesis turnaround (Hughes et al., 2017, Cold Spring Harbor perspectives in biology 9, a023812; Tang et al., 2016, Nature materials 15, 419). The toolbox has 56 promoter sequences that met this more stringent definition of non-repetitiveness (Table 1); 29 of them had higher transcription rates than the J23100 reference promoter (FIG. 2). Over 50 highly non-repetitive intrinsic transcriptional terminators and neutral DNA spacers were then identified using this more stringent definition of non-repetitiveness (L=12), leveraging existing toolboxes (Chen et al., 2013, Nature methods 10, 659) and bioinformatic design algorithms (Casini et al., 2014, ACS synthetic biology 3, 525-528). Altogether, these toolboxes of non-repetitive genetic parts are sufficient to express at least 29 transcriptional units without introducing more than 12 base pairs of repetitive DNA.

TABLE 1
Non-Repetitive Promoter Sequences

| SEQ | | |
|---|---|---|
| ID | | |
| NO: | Name | Sequence |
| 1 | pSH001 | TTTATAGGTTCACTGTAGAATCATACAATGGACTAA |
| 2 | pSH002 | TTTATGAGAGTATTCCTCCGATTTACAATGAGACTA |
| 3 | pSH003 | TTTATACGGTTCTTACGAAATAATACAATGGCTTTA |
| 4 | pSH004 | TTTATAGACTCCAGTAGTGTGGATACAATGCTAGCG |
| 5 | pSH005 | TTGACATGTTCCCAATAAGAGCAGACTATGCTTAGC |
| 6 | pSH006 | TTTATGGGACGGTTTATCAATACTACAATGCTTAGC |
| 7 | pSH007 | TTTATAACTTTACTACAGGGAGATACAATGACTAGC |
| 8 | pSH008 | TTGACAACAATCTGTAGCAGTTCGACTATGCTCTAG |
| 9 | pSH009 | TTTATACAATAAGTTCGTTGTCGTACAATGATCATA |
| 10 | pSH010 | TTTATATGACTTACCACTATTGGTACAATGGCCTAG |
| 11 | pSH011 | TTTATGGATTTTACCAACCGAGGTACAATGCCCTAA |
| 12 | pSH012 | TTTATGACTCGTAGCGTTCAGTATACAATGCCTGAG |
| 13 | pSH013 | TTGACAAAGAGATTTTCACTCGGGACTATGCTAGGG |
| 14 | pSH014 | TTTATGTTGAATAGTATCCACGCTACAATGCGGATA |
| 15 | pSH015 | TTTATATCGTCACACTGAAGAGTTACAATGTCTCAG |
| 16 | pSH016 | TTGACAGGGCAATAAATCGTTACGACTATGTCTAGC |
| 17 | pSH017 | TTTATATAGATAGCAGATTGACCTACAATGCATGTA |
| 18 | pSH018 | TTGACATGCGTTGAAACAGTAACGACTATGCAATAG |
| 19 | pSH019 | TTGACACCTGTGAGATTCATAGAGACTATGTCCTTA |
| 20 | pSH020 | TTTATGCGACTGATAACCTGTTGTACAATGCTCAGC |
| 21 | pSH021 | TTTATACTCAATACGGTGTCTGATACAATGTCGTAG |
| 22 | pSH022 | TTTATGCCACGATAAGTGTTACTTACAATGCTGCTA |
| 23 | pSH023 | TTGACAGAGTCAGAAACTTTACCGACTATGATCTAG |
| 24 | pSH024 | TTGACAGACTCGCAGTTTCAATAGACTATGCCTAGC |
| 25 | pSH025 | TTGACATATTACAACTCTGCTGAGACTATGCGTAGC |
| 26 | pSH026 | TTTATGAAGTTCTCTGAAACAGATACAATGCTAGC |
| 27 | pSH027 | TTTATATTCAGACTCGGTATAGGTACAATGCTAGC |
| 28 | pSH028 | TTGACACTGTAACTGCGAATAGAGACTATGCTAGC |
| 29 | pSH029 | TTTACGCCGTGAAGTAATACAGATACTATGCTAGC |
| 30 | pSH030 | TTTACGAAGGAACTGTCTATAGGTACAATGCTAGC |
| 31 | pSH031 | TTTATGACTTTCGTAGGCATAGATACAATGCTAGC |
| 32 | pSH032 | TTTATAAGCAACTTCGGTATAGGTACAATGCTAGC |
| 33 | pSH033 | TTGACATGGCTGTATCACATAGGGACTATGCTAGC |
| 34 | pSH034 | TTTACGTCGTTATCAGCGACCGATACTATGCTAGC |
| 35 | pSH035 | TTTACGTACTGGTGAACTATAGGTACAATGCTAGC |
| 36 | pSH036 | TTGACATGACTCTCCAGCTGTGCTATAATTGTACT |
| 37 | pSH037 | TTGACATTTCGTCAAGAGTCGACTATAATATCGCG |
| 38 | pSH038 | TTGACATGAGCTCGTCGTCAGGATATATAGCTTT |
| 39 | pSH039 | TTGACATGAAGTGTTAGACGTCATATAATCGTGGT |
| 40 | pSH040 | TTGACATAGGCAAGCCAGTATAGTATAATCACATA |
| 41 | pSH041 | TTGACAGTCCTCGAACACCTCTATATAATAGTGTC |
| 42 | pSH042 | TTGACAGTAGATCAGAGGGTTGCTATAATCGACAG |
| 43 | pSH043 | TTGACACTACCGAGACAGTGACATATAATAGGACC |
| 44 | pSH044 | TTGACACGATGCTTGCTGCTACCTATAATAACATA |
| 45 | pSH045 | TTGACAACTGCTCAGCGAAATACTATAATGACTAC |
| 46 | pSH046 | TTGACAGGTGAACGCTCAGCTCTTATAATGCCTAT |
| 47 | pSH047 | TTGACACTGGCCTGACAAGTCCATATAATGATGTC |
| 48 | pSH048 | TTGACACTATGGTCCGCAAGCATTATAATGCTCTG |
| 49 | pSH049 | TTGACAAAGTACTACTGTATTAGTATAATTGTCAT |
| 50 | pSH050 | TTGACATGCGTGATTTAACATTCTATAATTGCACA |
| 51 | pSH051 | TTGACATAAGTCGTATTCAAAGATATAATATAGGT |
| 52 | pSH052 | TTGACAGTTGTGTTATCCGGCCATATAATATCTCT |
| 53 | pSH053 | TTGACAGTGTGCTAAAATTTGTCTATAATGAGTAC |
| 54 | pSH054 | TTGACAGCATCTGCTTTGTCACCTATAATTCAATG |
| 55 | pSH055 | TTGACAGACCTTATCTACATGGTTATAATCTGAAT |
| 56 | pSH056 | TTGACACTTTGCACATGTCCCGTTATAATCATGAT |
| 57 | pSH057 | TTGACACGGATCTTCGCTGAACGTATAATGAGAAA |
| 58 | pSH058 | TTGACACAGCCCAGCCGGAGAGTATAATCCTATT |
| 59 | pSH059 | TTGACAATCGCTGTCTACGTGAATATAATGAATTT |
| 60 | pSH060 | TTGACATTAGCACTTGAGCTGATTATAATGGGCCG |
| 61 | pSH061 | TTGACAGAGGCAGTACTACCGTTTATAATTCGGAC |
| 62 | pSH062 | TTGACACCTCATCTTATAGTTCCTATAATTTCTAT |
| 63 | pSH063 | TTGACACCGGGTTGAATACTATCTATAATGTACGG |
| 64 | pSH064 | TTGACACATTAGGATGGACGTATTATAATATGCCC |

Next, a toolbox of highly non-repetitive sgRNA handles capable of being loaded into Cas9sp to form active ribonucleoprotein (RNP) complex was designed and characterized. Currently, when multiple sgRNAs are expressed, they all use the same 61-nucleotide handle sequence, which is a fusion between the wild-type crRNA and tracrRNA, and often includes the tracrRNA's wild-type transcriptional terminator. A rational strategy was employed to design many sgRNA handles with maximally different nucleotide sequences, while maintaining their functionality. In this approach, RNA structure prediction (Lorenz et al., 2011, Algorithms for Molecular Biology 6, 26) and Monte Carlo optimization were applied to generate mutated sgRNA handle sequences that all satisfy a proposed sequence and structural design constraint. From this large set of candidate sgRNA handles, non-repetitive sgRNA handles with a desired maximum shared repeat length were identified and characterized (Table 2).

TABLE 2
Non-repetitive sgRNA handles

| SEQ ID | |
|---|---|
| NO: | Sequence |
| 65 | GTTTTAGAGCTAGAAATAGCAAGTTAAAATAAGGCTAGTCCGTTATCAACTTG |
| | AAAAAGTG |
| 66 | GTTCTAGAGCTCGAAAGAGCAACTTAGAATAAGCCTAATCCCTGATCAACTTG |
| | AAAAAGTC |
| 67 | GTTCTAGAGCTGGTAACAGCAAGTTAGAATAAGTCTAGTCCATTATCAACTGG |
| | AAACAGTG |
| 68 | GTTTTTGAGCGAGAAATCGCAAGTAAAAATAAGGCTCGTCCGTTAACAAGTTG |
| | AAAAACTG |
| 69 | GTTTTATAGCTAGAAATAGCAAGATAAAATAAGGCTAGTCCATTATCAACTTG |
| | AAAAAGTG |
| 70 | GTTTTGCAGCTAGAAATAGCAAGGCAAAATAATGCTAGTCCGTTCCCAACTTG |
| | AAAAAGTG |
| 71 | GTTTTAGATCACGAAAGTGAAAGTTAAAATAAGCCTAGCCCGTTACCAACTGG |
| | AAACAGTG |
| 72 | GTTTTGGAGCTAGAAATAGCAAGTCAAAATAAGGCTAGTCCGTTCTCAACTTG |
| | AAAAAGTG |
| 73 | GTTTTAGAGATGGAAACATCAAGTTAAAATAAGGCAAGTCCGTTAACAACTCG |
| | AAAGAGTG |
| 74 | GTGTTAGAGTTGGAAACAACAAGTTAACATAAGGCTACTCGGATTTCAACGTG |
| | AAAACGTC |
| 75 | GTTTTAGAGCTAGCAATAGCAAGCTAAAATAATGCTAGTCCGTTATTAACTTG |
| | AAAAAGTG |
| 76 | GGTTTAGAGTTAGAAATAACAAGTTAAACTAAGGCTAGTCCGTTATAAACTTG |
| | AAAAAGTC |
| 77 | GTTTTAGAGCTTGAAAAAGCAAGTTAAAATTAGGCTAGTCCGTTAACAACTTG |
| | AAAAAGTG |
| 78 | GTATTAGAGCTAGAAATAGCAACTTAATATAAGGCTAGTCGGTTATCACCTTG |
| | AAAAAGGG |
| 79 | GTTTTCGAGCTAGTAATAGCAAGTGAAAATGAAGTTAGTCCGTTAGCAAACTG |
| | AAAAGTTA |
| 80 | GTTGTAGATCTAGAAATAGAATGTTACAATTAGGCTAGTCCGTTATGAACATG |
| | AAAATGTG |
| 81 | GTTTGAGAGATCGAAAGATCAAGTTCAAACAAGTCTAGTCCGTTGTGAACCTG |
| | AAAAGGTG |
| 82 | GTTTTAGAGCTACACATAGCAAGTTAAAATAAAGGTAGTCCGTTATCAGTTTG |
| | AAAAAACG |
| 83 | GTTGTAGAGCTAGAAATAGCGAGTTACAATAAGGCTAGTCCGTTATGAACTTG |
| | AAAAAGTG |
| 84 | GTTTTAGAGTGAGAAATCACAAGTTAAAATAAGGCTAGACCGTTATCAACTAG |
| | AAATAGTG |
| 85 | GTTTAAGGGTTAGAAATAACAAGTTTAAATAAGGCAAGTCCGTTATCAAGTGG |
| | CAACACTC |
| 86 | GCTTTAGACCTTGAAAAAGGAAGTTAAAGTAAGGCTAGTCCGTTATGACCTTG |
| | AAAAAGGG |
| 87 | GTTTTACACCTAGAAATAGGAAGGTAAAATAAGGCTGGTCCGTTATCACCTCG |
| | AAAGAGGG |
| 88 | GTTGTAGAGCTAGCAATAGCAGGTTACAATAAGGCTCGTCCGTTATAAACATG |
| | AAAATGTG |
| 89 | GATTTCGAGCTAGGCATAGCAAGTGAAATTAAGGCTGGTCCATTAACACCTTG |
| | AAAAAGGG |
| 90 | GCTTTACAGCTAGAAATAGCAGGGTAAAGTAAGGCTAGTCCGTAATAAACGTG |
| | AAAACGTG |
| 91 | GTTTCAGAGCAAGAAATTGCAAGTTGAAATAAGGCTAGTCCGTTAAAAACTTG |
| | AAAAAGTG |
| 92 | GTATCTGAACTCGACAGAGTAAGTAGATATAAGGCCAGTCCGTTAGCAACTTG |
| | AAAAAGTC |
| 93 | GTTTTAGACCTAGAAATAGGAAGTTAAAATAAGGCTAGTTCGTTATCATCTTG |
| | AAAAAGAG |
| 94 | CTTTTAGAGATAGAAATATCAAGTTAAAAGAAGGCTAGTCCGTTACCAACTTG |
| | AAAAAGTG |
| 95 | GATTTAGAGCTGGAAACAGCAAGTTAAATTAAGGCTAGTCCGTTATCAGCTTG |
| | AAAAAGCG |
| 96 | GTTGTAGAGGAAGAAATTCCAAGTTACAATGAGGCTAGTCCGTGATGAACTTG |
| | AAAAAGTG |
| 97 | GCTTTATATCTAGAAATAGAGAGATAAAGTAAGGCAAGTCCGTTATCATCTGG |
| | AAACAGAC |
| 98 | GTTTAAGAGCTAGAAATAGCACGTTTAAATAAGGCTAGTCCGTTTTCAACTTG |
| | AAAAAGTG |
| 99 | GTTTTACAGCTAGTGATAGCAAGGTAAAATAAGGCTAGTCCCAAATCAACTTG |
| | AAAAAGTG |
| 100 | GCTTTAGAGCTAGAAATAGCAGGTTAAAGTAAGGCCAGTCCGTAATAAACTGG |
| | AAACAGTG |
| 101 | GTGTTAGAGTCAGATATGACATGTTAACATTAGGCTAGTCCGGGGTGAAGTTG |
| | AAAAACTG |
| 102 | TTACTAGAGTGACAAATCACAAGTTAGTAAAAGGCTAGACCGTTATAATCCCG |
| | AACGGGAG |
| 103 | TTTTCAGATTTGGAAACAAAACGTTGAAAAAAGGCAAGTCCGTTATGAACGCG |
| | AAAGCGTG |
| 104 | GTATGCGAGGTAGAAATACCCAGTGCATATCAGGCTAGTCCGATATCATGTTG |
| | AAGAACAG |
| 105 | CGGTTAGGATAAGAAATTATAAGTTAACCGTAGGCTAGCCCGTTATAAACTGG |
| | AAACAGTG |
| 106 | GATGTAGATGTAGAAATACAAGGTTACATTAAGGCCCGTCCGTAATCAACTTG |
| | AAGAAGTG |
| 107 | GTTTTGGACCTAGAAATAGGAAGTCAAAATAAGGCTGGACCGACATGTAATCG |
| | AAAGATTT |
| 108 | CCTTAAGAGCTAGCAATAGCAAGTTTAAGGAAGGCAAGCCCGTTATCATCCTG |
| | AATAGGAC |
| 109 | GTAATAGAGATGGATACATCAAGTTATTATAAGGCTCGACCGTTAACAGTCTG |
| | AAAAGACG |
| 110 | GTTGGAGAGCAAGACATTGCAAGTTCCAATAAGGCGTGTCCGATAAAAGCTTG |
| | AGAAAGCA |
| 111 | ATCTGAGAGCCAAAAATGGCAAGTTCAGATAAGGCCAGACCGTTACCAGCTTA |
| | AATAAGCG |
| 112 | GCTTCAGATCCAGAAATGGAAAGTTGAAGTGAGGCAGGTCCGGTAGCAACTC |
| | GAAAGAGTG |
| 113 | AGTTTAGAGAATGCAAATTCAAGTTAAACTAAGGCGAGTCCGGTATAATCGTG |
| | TAAACGAG |
| 114 | GGAATAGAAAACAAAAGTTTAAGTTATTCTAAGGCCAGTCCGGAATCATCCTA |
| | AAAAGGAG |
| 115 | GTGCTAGAGTCGTAAACGACAAGTTAGCATTAGGCTTGTCCGCAATGAACCTG |
| | AAAAGGTG |
| 116 | CATTTTGGCGTCGAAAGACGAAGTAAAATGAAGGCGAGACCGATATCAACTG |
| | GAAGCAGTG |
| 117 | TTTTTAGAGGAAGGAATTCCAAGTTAAAAAAAGGCAGGACCGGGAACATGTT |
| | GAAAAACAG |
| 118 | CTTACCGAACTAGGAATAGTAAGTGGTAAGAAGGCCTGACCGTAATAAGCCTG |
| | AAAAGGCG |

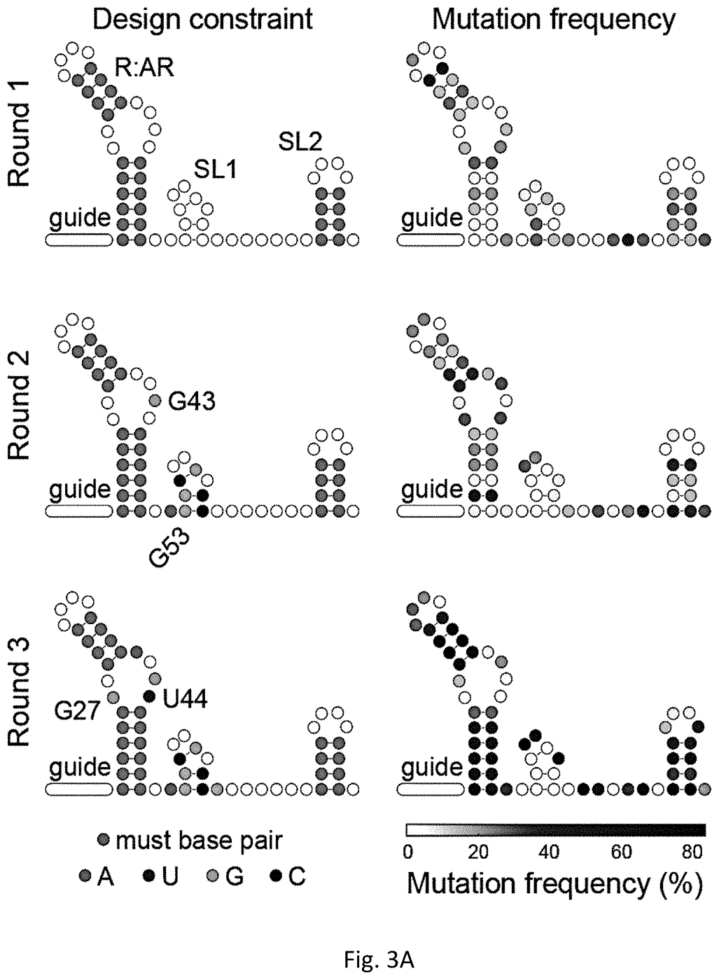

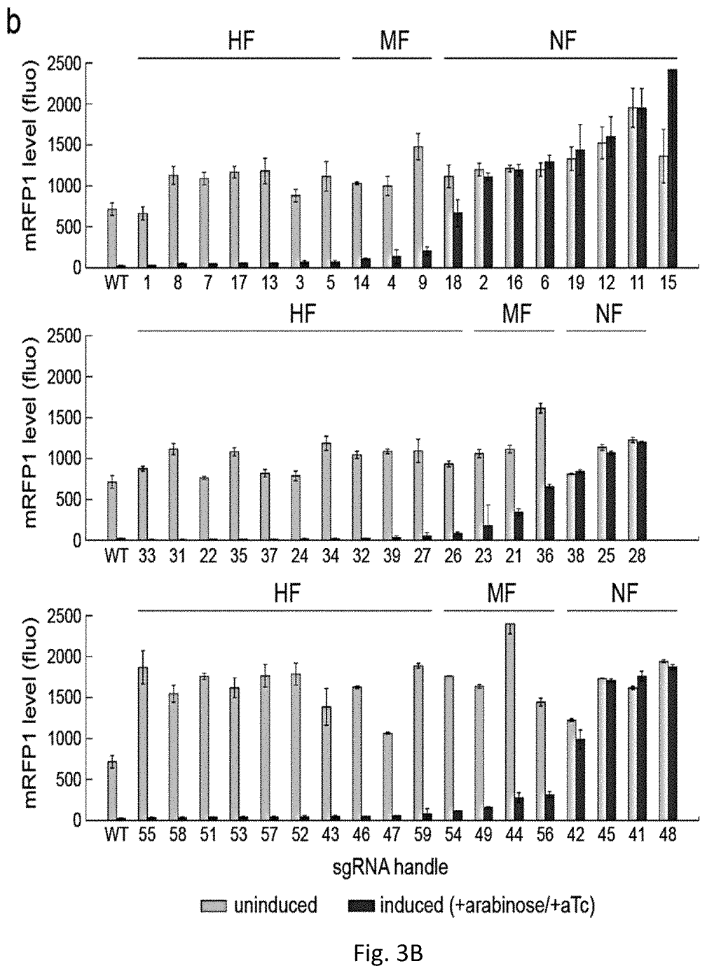

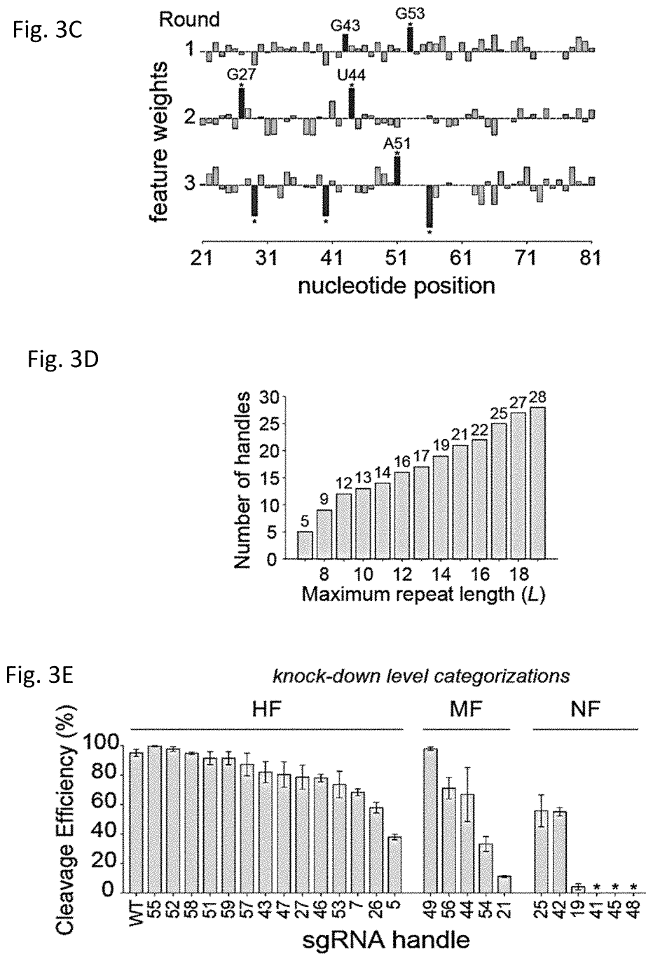

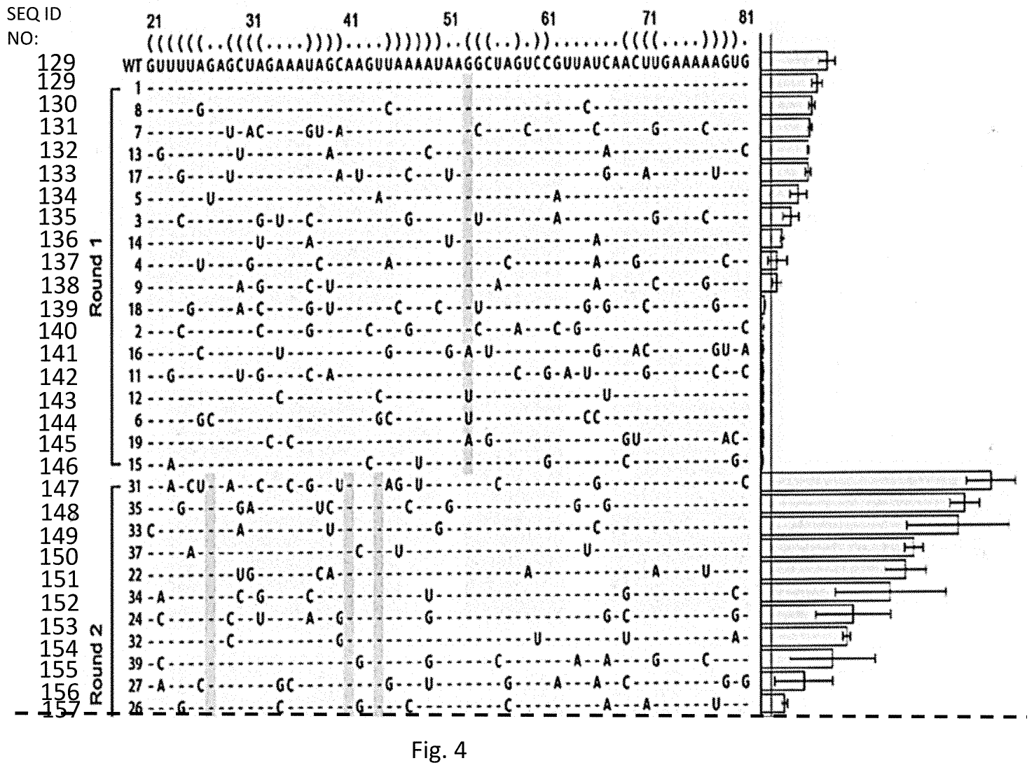

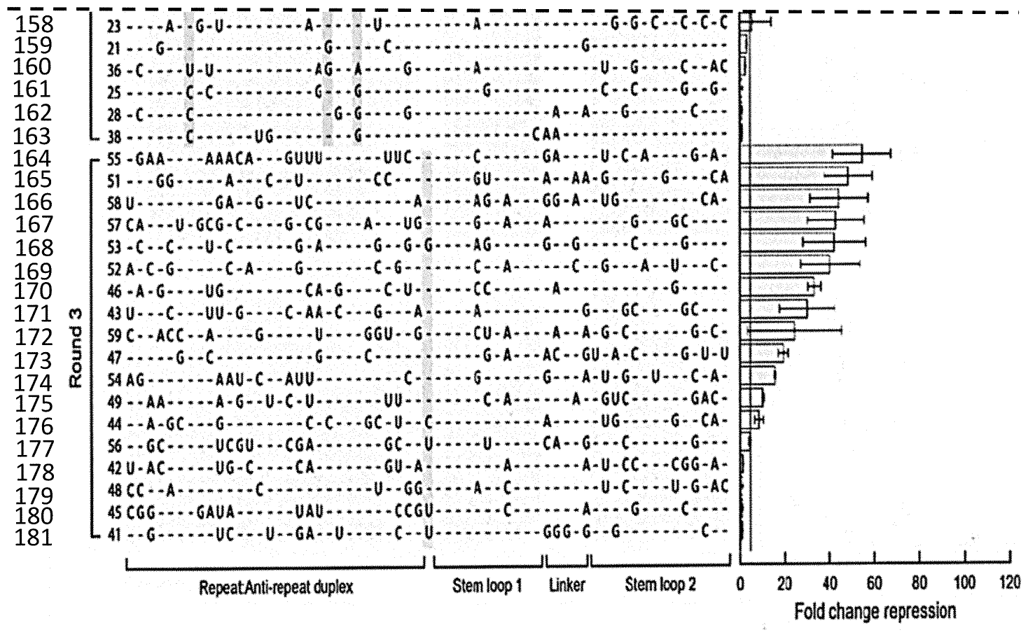

Based on structural and biochemical data (Jiang et al., 2016, Science 351, 867-871; Jinek et al., 2014, Science 343, 1247997; Briner et al., 2014, Molecular cell 56, 333-339; Nishimasu et al., 2014, Cell 156, 935-949), it was initially hypothesized that functional sgRNA handles must fold into the wild-type RNA structure (FIG. 3A). In the first round, 18 sgRNA handle sequences were designed, containing an average of 7 mutations and a maximum shared repeat of 20 nucleotides (FIG. 4). The ability to knock-down expression of a plasmid-encoded mRFP1 reporter protein within an E. coli strain that uses inducible plasmid-encoded expression of deactivated Cas9sp (dCas9sp) (Nielsen et al., 2014, Molecular systems biology 10, 763) was measured. 7 diversified sgRNA handles were labeled highly functional as they knocked down mRFP1 expression between 10 to 25-fold, comparing induced to non-induced cells maintained in the exponential growth phase (FIG. 3B). To compare, the wild-type sgRNA handle knocked down mRFP1 expression by 30-fold under the same conditions. The remaining sgRNA handles were labeled either moderately functional if they somewhat knocked down reporter expression (7-9 fold) or non-functional if they displayed no knock-down effect. Overall, the data indicated that over 50% of the sgRNA handle's nucleotide positions could be mutated without compromising function (FIG. 4), though it was clear that the initial design constraint could be improved by further specifying the nucleotide positions that make essential contacts with Cas9sp.

Next, machine learning was applied to successively improve the design constraint across three rounds of a design-build-test-learn cycle, using linear discriminant analysis (LDA) to identify the mutated nucleotide positions that were associated with breaking sgRNA handle function. From the round 1 dataset, LDA determined that mutating nucleotides G43 and G53 resulted in greatly reduced knock-down activity (FIG. 3C). In round 2, the design of non-repetitive sequences was repeated, using an improved design constraint that prevented mutation of G43, G53, and modifications to the non-canonical structure in SL1. 17 non-repetitive sgRNA handle variants were selected with an average of 8 nucleotide mutations and their ability to knock-down mRFP1 expression in E. coli was characterized. 11 diversified sgRNA handles were highly functional, knocking down mRFP1 expression between 10 and 102-fold (FIG. 3B). From the round 2 dataset, LDA determined that mutating nucleotides G27 and U44 resulted in lower knock-down levels. These essential nucleotides were incorporated into the design constraint and then the rational design approach was repeated. For round 3, the number of mutations was doubled, and an even greater degree of non-repetitiveness was specified with a maximum shared repeat of only 12 nucleotides. 18 of these diversified sgRNA handle sequences were selected for characterization, and it was found that over 66% of them were highly functional, even though they were heavily mutagenized, with an average of 17 mutated positions spread evenly across the R:AR, SL1, and SL2 hairpins (FIG. 3A and FIG. 3B). Using the round 3 dataset, LDA could identify only one more essential nucleotide at A51 (FIG. 3C).

Overall, 28 highly functional, highly diversified sgRNA handle sequences were designed and characterized that can be collectively co-expressed for many-gene regulation. This toolbox is non-repetitive with an L of 20 nucleotides, enabling them to be integrated together into a single genomic loci without introducing genetic instability; the chance of triggering homologous recombination is about 1 in 50,00022. 16 of these diversified sgRNA handles are even more non-repetitive (L=12), enabling them to be readily synthesized together within a single non-clonal DNA fragment with a quick turnaround time (FIG. 3D).

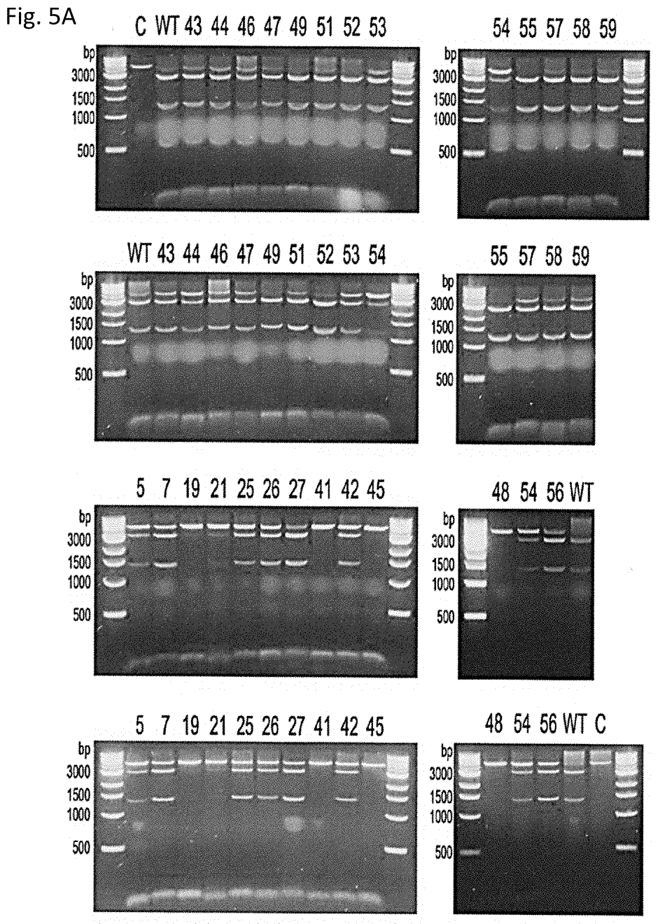

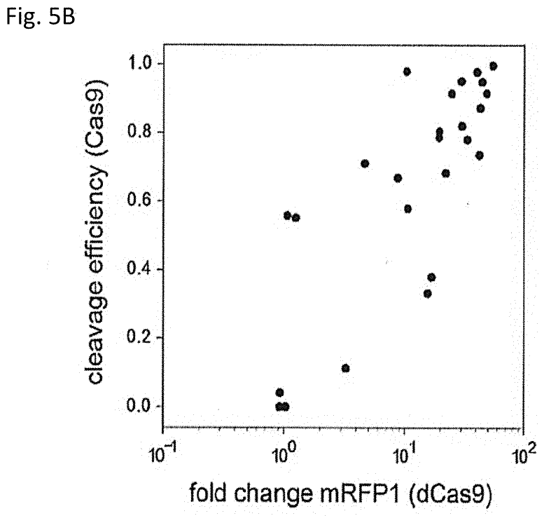

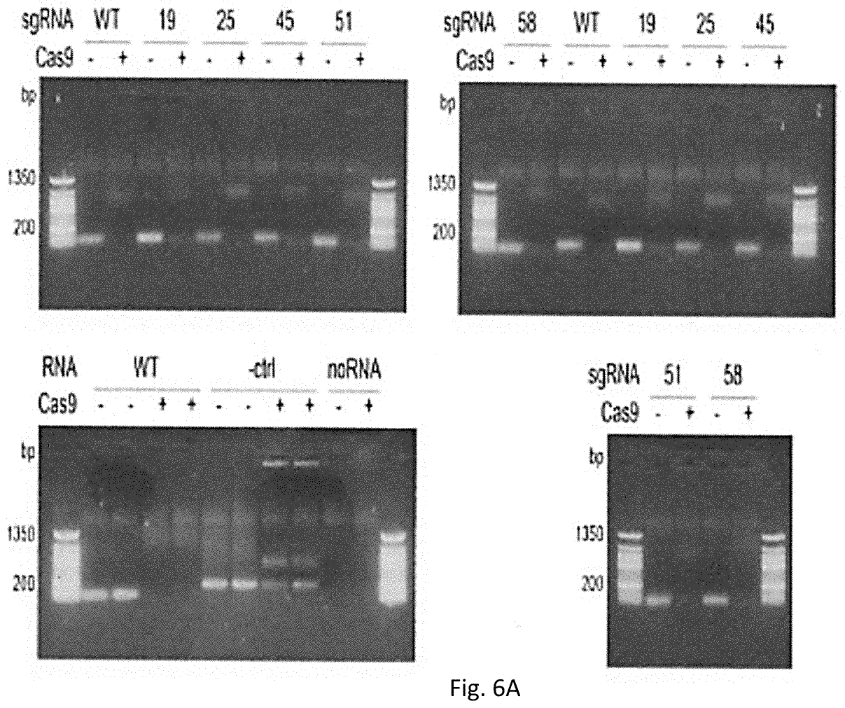

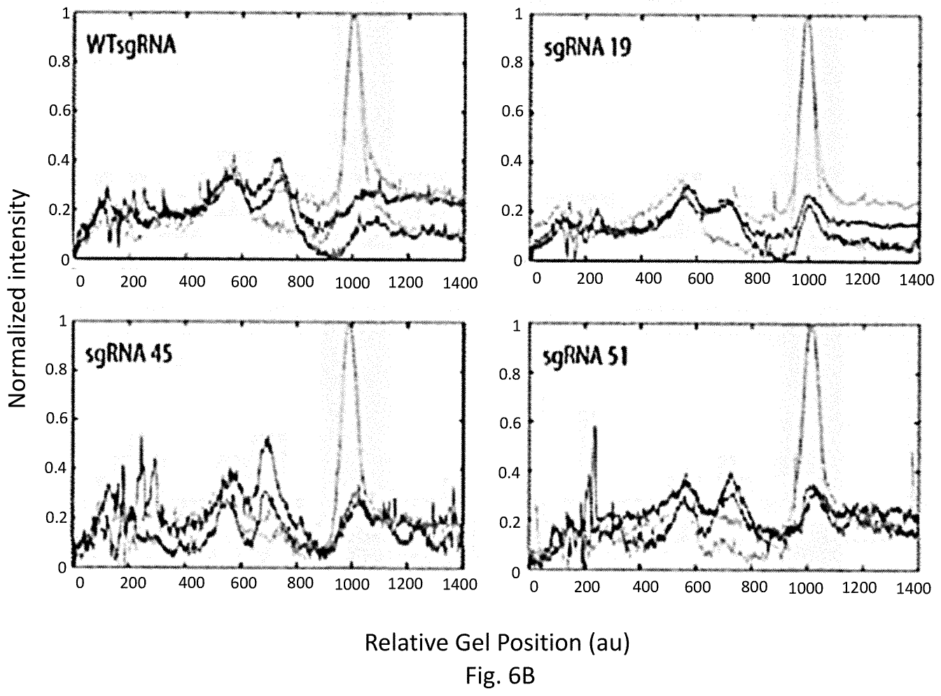

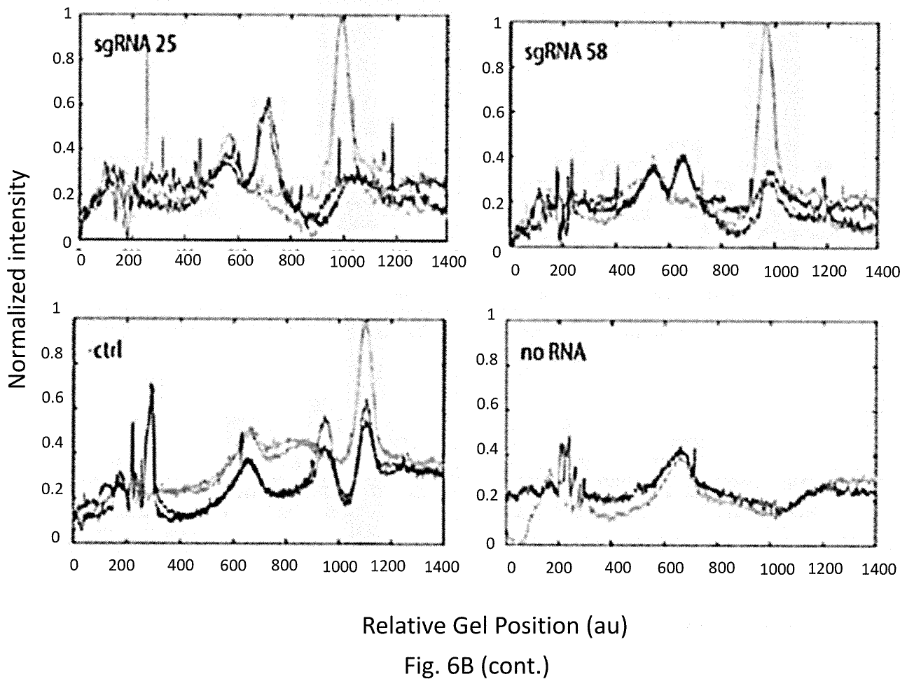

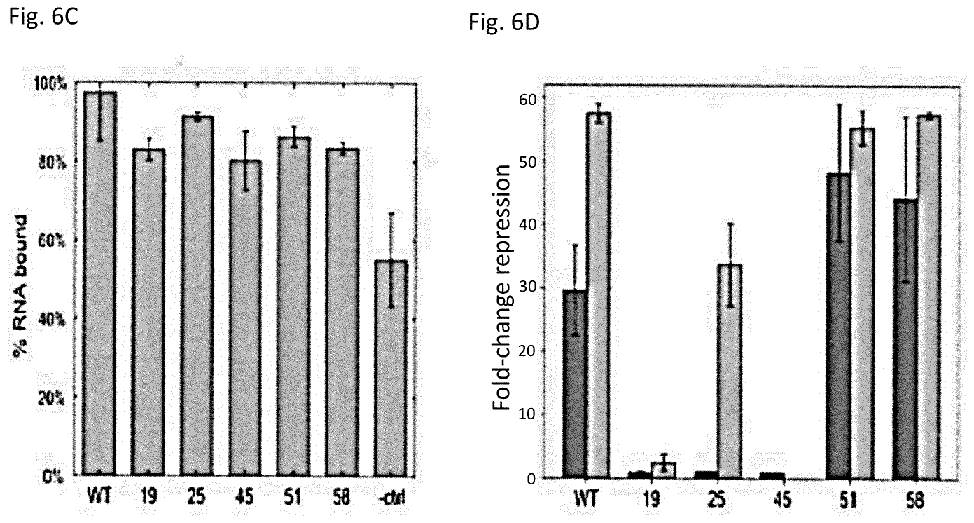

Without being bound by theory, it was hypothesized that the non-repetitive sgRNA handles could also be used with endonuclease active Cas9sp to cleave DNA sites. 15-minute cleavage assays were performed on DNA templates using 26 diversified sgRNA handles, including the wild-type. With a few exceptions, high correspondence was found between a handle's ability to repress mRFP1 expression and its cleavage efficiency (FIG. 3E, FIG. 5A and FIG. 5B). Relatedly, without being bound by theory, it was hypothesized that non-functional sgRNA handles could not bind or cleave DNA because the handle mutations could disrupt their ability to load into Cas9_{SP}, which is an essential step towards forming active RNP complex (Jinek et al., 2014, Science 343, 1247997). Electrophoretic mobility shift assays were performed to measure the fraction of Cas9_{SP} and sgRNA that can self-assemble into RNP complex at equilibrium within a buffered solution. Surprisingly, only small differences were found in RNP complex formation using either the highly functional or non-functional sgRNA handles from the toolbox (80-90% bound), compared to a wild-type handle sequence (˜98% bound) and a non-CRISPR structured RNA used as a negative control (55% bound) (FIG. 6A through FIG. 6D), suggesting that the RNA sequence-structure features responsible for loading into Cas9sp had not been disrupted. Altogether, the data show that the highly functional, diversified sgRNA handles bind to (d)Cas9sp and mediate either transcriptional knock-downs or DNA cleavage (Dagdas et al., 2017, cience advances 3, eaao0027). In contrast, the non-functional sgRNA handles bind to Cas9sp, but are incapable of correctly guiding Cas9sp to cognate DNA sites, for example, by disrupting the conformational switch in apo-Cas9 that enables them to unwind PAM-containing DNA templates (Anders et al., 2014, Nature 513, 569) or by preventing the formation of stable R-loops during the binding process (Farasat et al., 2016, PLoS Computational Biology 12, e1004724).

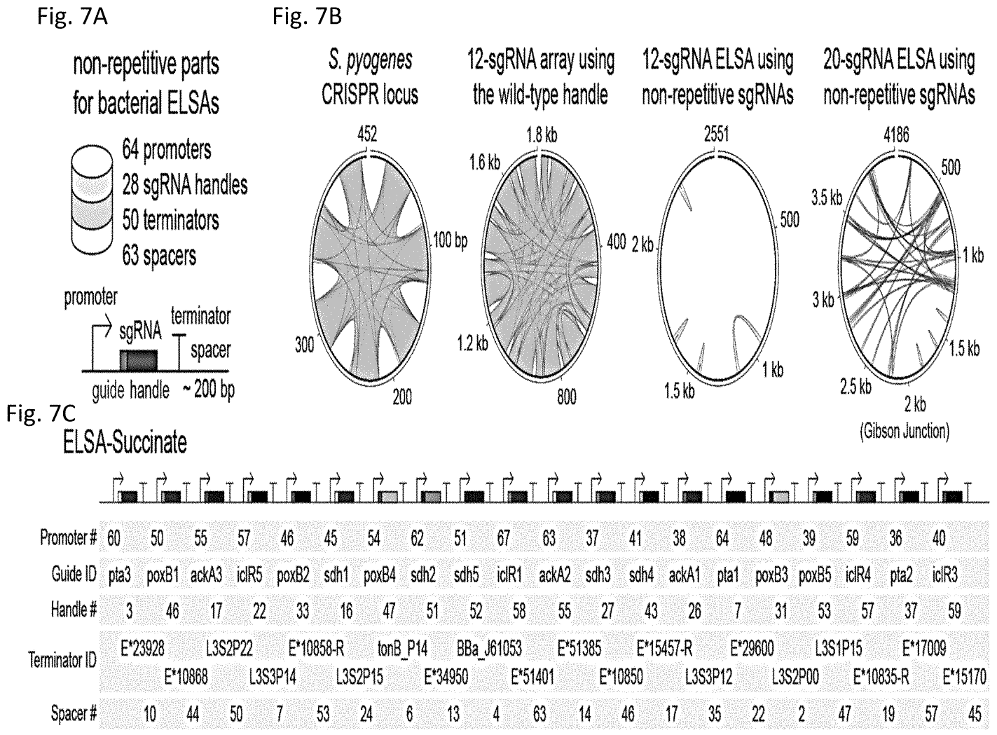

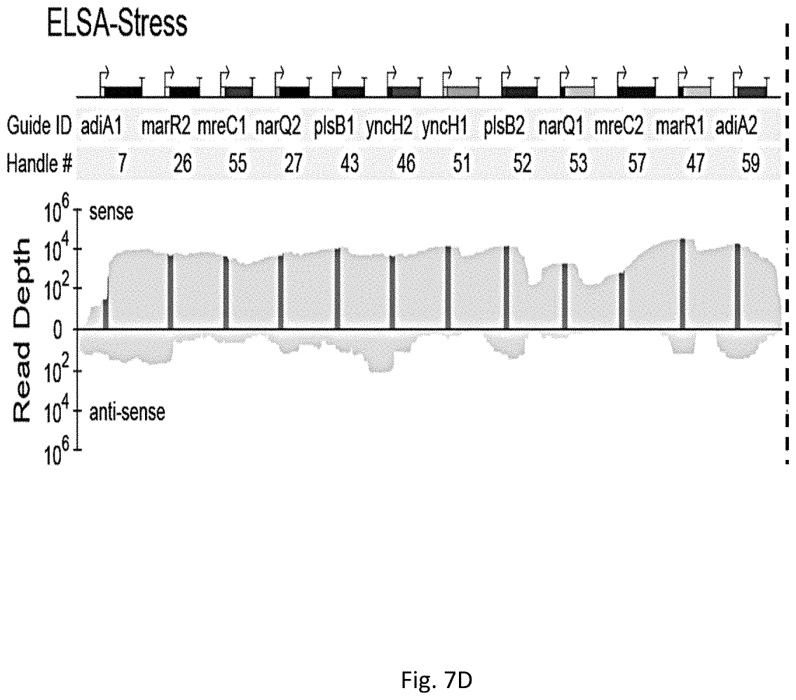

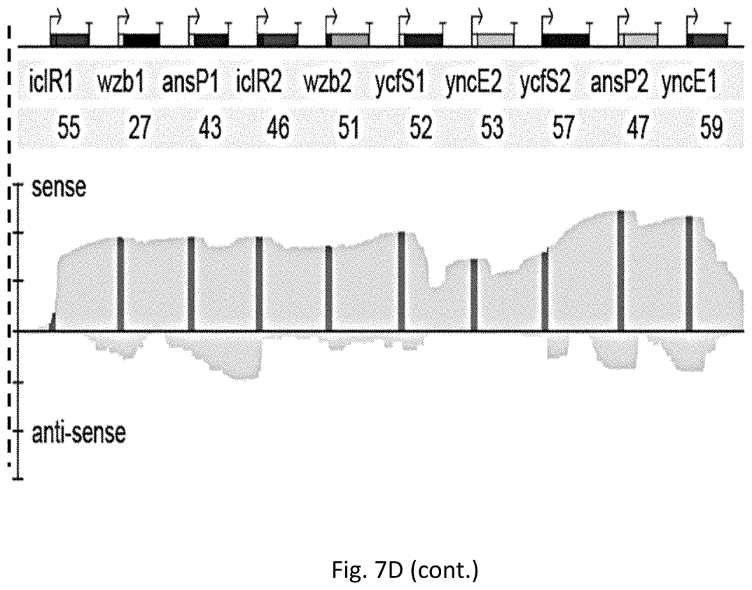

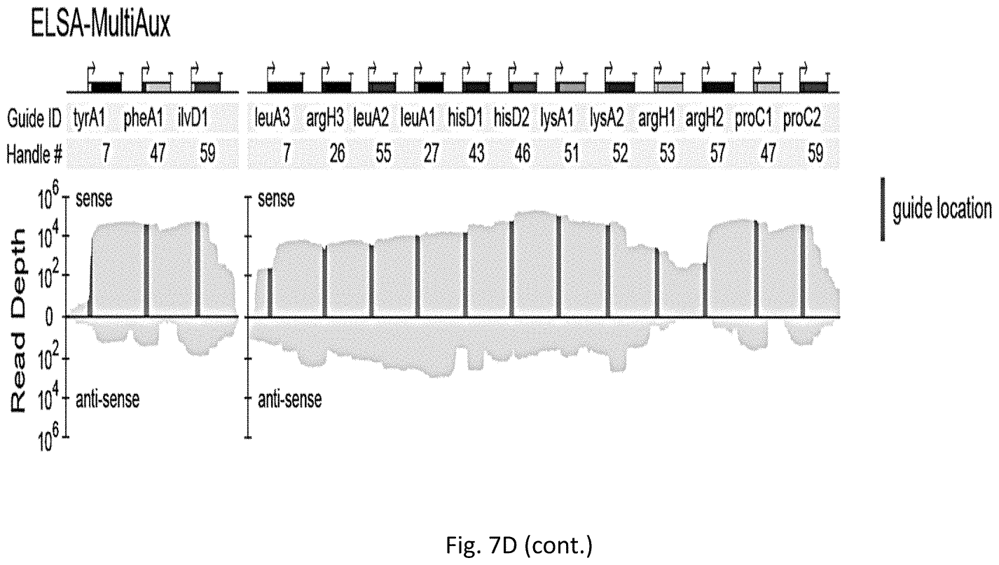

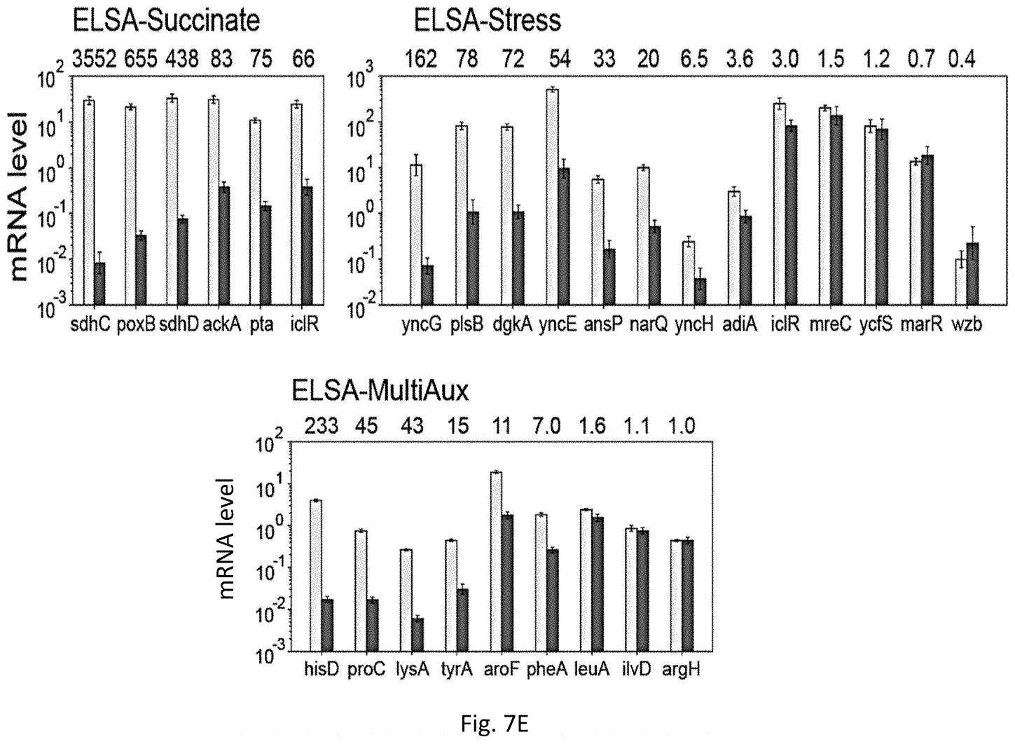

An integrated computational-experimental workflow was developed to co-express up to 22 sgRNAs within extra-long sgRNA arrays (ELSAs) (FIG. 7A through FIG. 7E). The targeted genomic regions are inputted into an optimization algorithm, called the ELSA Calculator, that selects the sgRNAs' guide sequences, identifies the optimal ordering of genetic parts, and generates an ELSA sequence (Methods). ELSAs are designed using the toolboxes of non-repetitive genetic parts (FIG. 7A) to maximally satisfy 23 design rules. Together, the algorithm (i) eliminates candidate guide RNA sequences predicted to have substantial off-target binding activity, according to a biophysical model of CRISPR/Cas9 activity, called the Cas9 Calculator (Farasat et al., 2016, PLoS Computational Biology 12, e1004724); (ii) minimizes mis-hybridization events during DNA fragment synthesis via ligation assembly or polymerase cycling assembly (Hughes et al., 2017, Cold Spring Harbor perspectives in biology 9, a023812); (iii) removes polymeric sequences prone to DNA replication error (Jack et al., 2015, ACS synthetic biology 4, 939-943); and (iv) minimizes the reduced expression of sgRNAs by premature transcriptional termination or anti-sense RNA expression (Brophy et al., 2016, Molecular systems biology 12, 854). Prior to the algorithm's development, manually designed ELSAs had a high risk of synthesis failure and contained several undesired genetic elements, particularly internal anti-sense promoters.

Using the ELSA calculator, a 4186 bp ELSA was designed co-expressing 20 sgRNAs utilizing 100 non-repetitive genetic parts (promoters, sgRNA guides, sgRNA handles, transcriptional terminators, and neutral DNA spacers) with a maximum shared repeat of only 16 base pairs. The algorithmic design enables rapid construction and genome-integration of this complex genetic system. The two synthesized DNA fragments were used to build the integration vector in a 3-part Gibson assembly, and employed the pORTMAGE system (Nyerges et al., 2016, Proceedings of the National Academy of Sciences 113, 2502-2507) to insert the integration cassette into the E. coli genome with an overall design-to-test time of about 14 days. In contrast, because of their highly repetitive DNA sequences, the same facile workflow could not be applied to building the natural S. pyogenes CRISPR locus (containing seven 36 bp repeats) or a 20-sgRNA ELSA that repeatedly used the original sgRNA handle (containing twenty 61 bp repeats) (FIG. 7B).

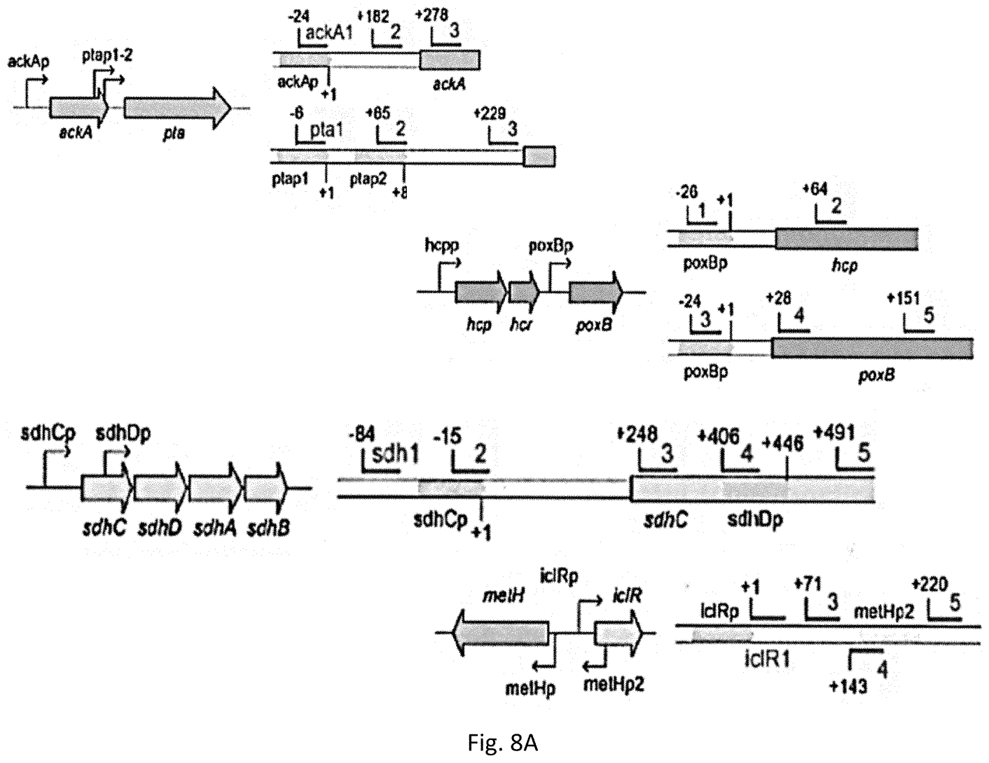

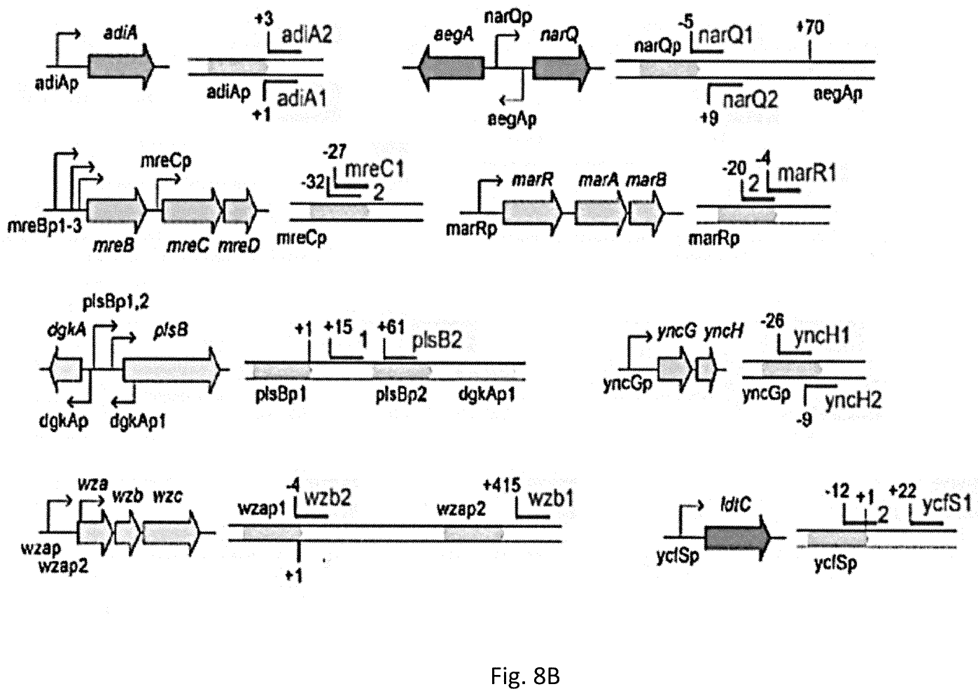

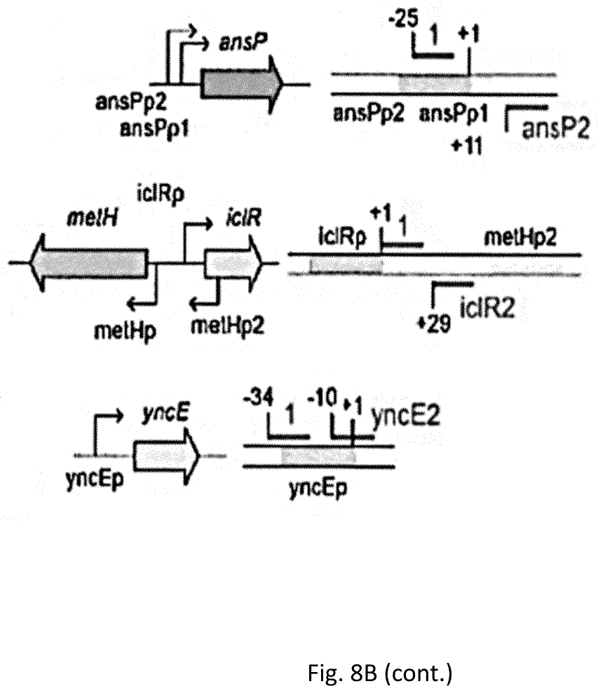

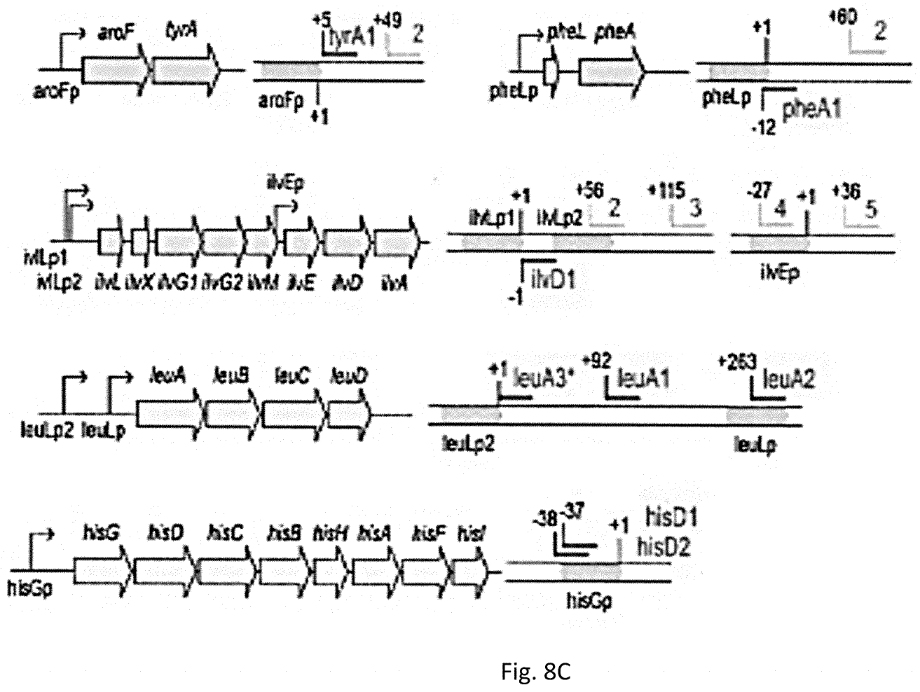

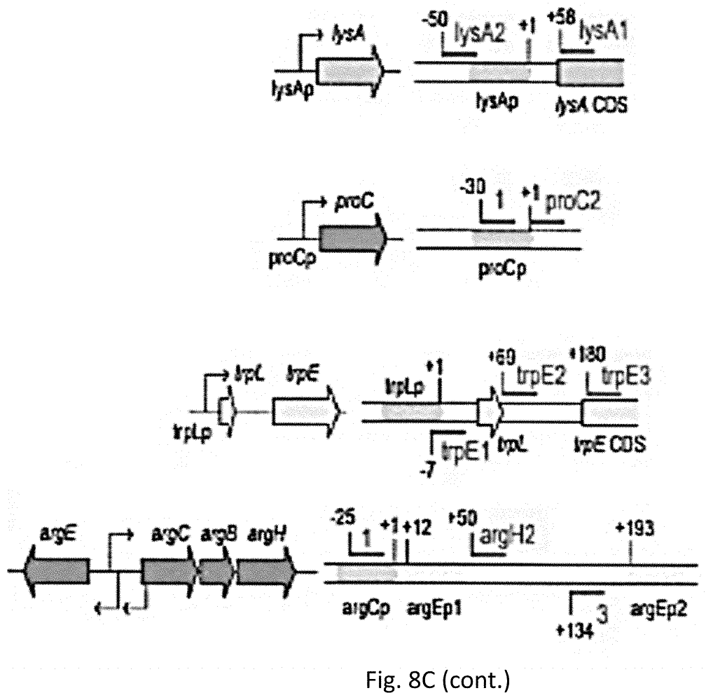

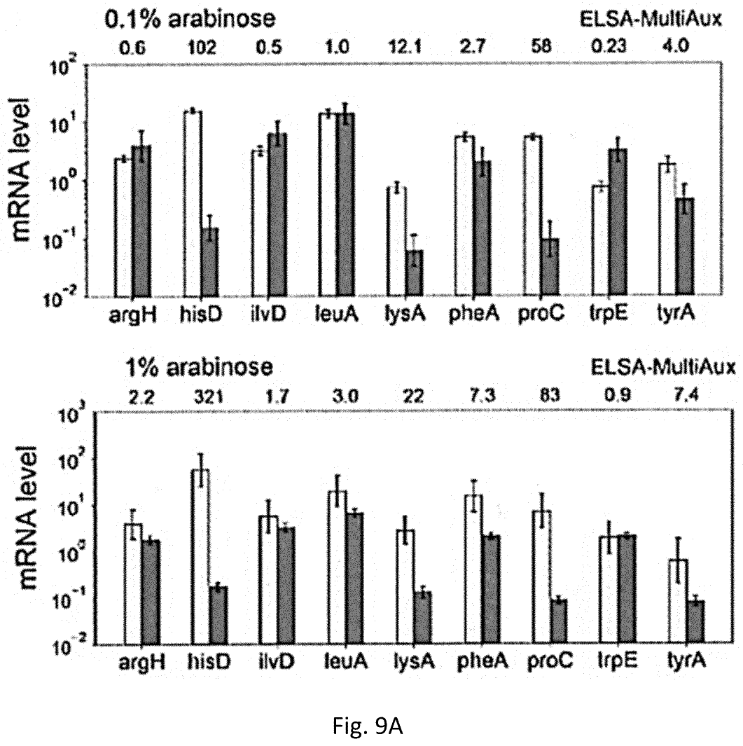

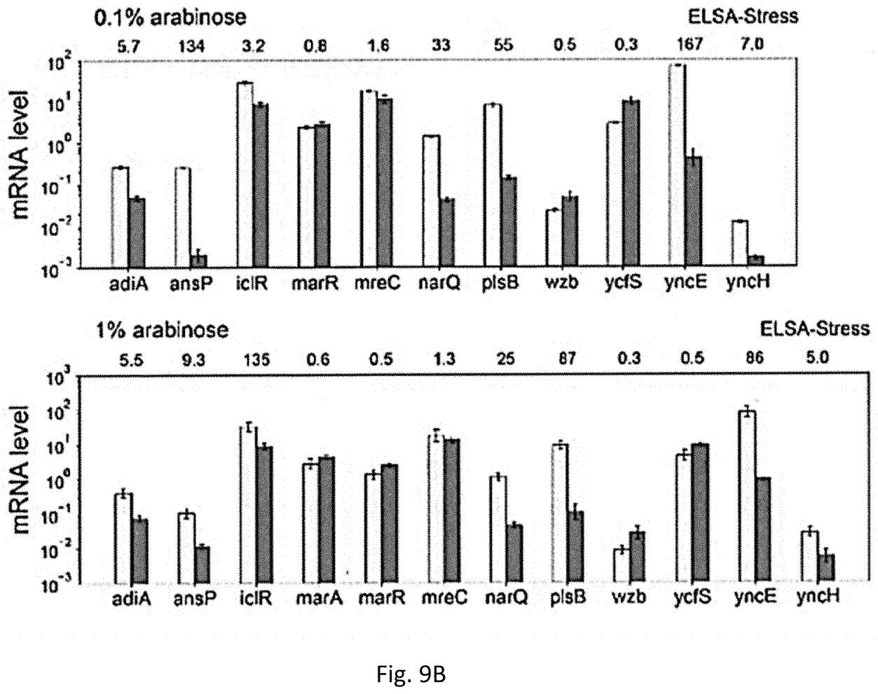

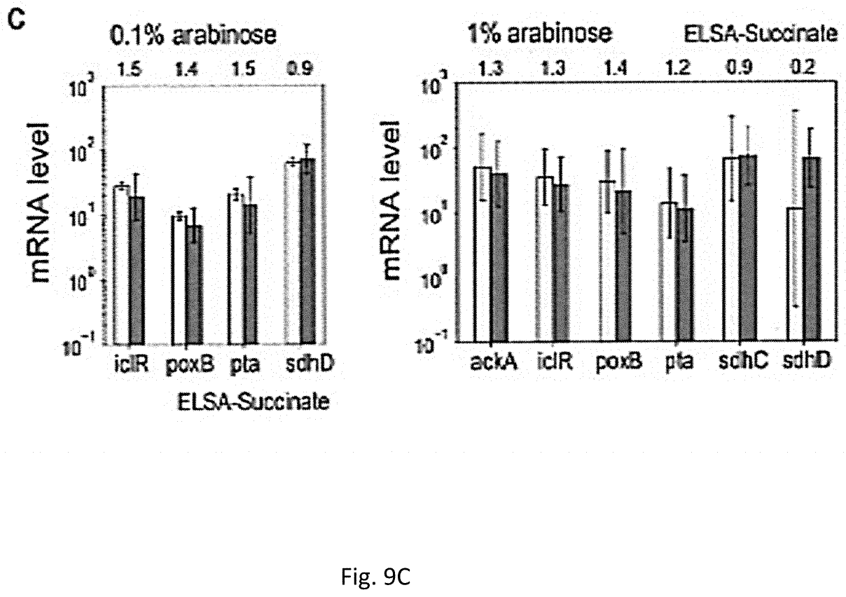

This workflow was used to design, build, and characterize ELSAs for three demonstrative applications (FIG. 7C and FIG. 7D). In the first example, a 20-sgRNA ELSA (ELSA-Succinate) was designed to simultaneously knock-down the expression of 6 genes (ackA, ic1R, poxB, pta, sdhC, sdhD), necessary for E. coli to over-produce succinic acid (Lin et al., 2005, Biotechnology and bioengineering 89, 148-156). This host E. coli strain (SJ_XTL219) expresses deactivated Cas9sp using an arabinose-inducible promoter, enabling inducible transcriptional knock-downs (Li et al., 2016, Scientific reports 6, 39076). Multiple sgRNAs were expressed per gene to repress transcriptional initiation at all known promoters driving gene expression. Additional sgRNAs were also expressed to knock-down expression during transcriptional elongation (FIG. 8A through FIG. 8C). Initially, the targeted genes were not appreciably knocked-down (1.4-fold maximum), according to RT-qPCR measurements (FIG. 9A through FIG. 9C). With so many sgRNAs expressed, it was hypothesized that there was an insufficient concentration of dCas9sp inside the cell to fully mediate transcriptional repression, creating a scarce shared resource (Chen et al., 2018, bioRxiv). Therefore, the RBS Library Calculator (Farasat et al., 2014, Molecular systems biology 10, 731) and pORTMAGE (Nyerges et al., 2016, Proceedings of the National Academy of Sciences 113, 2502-2507) were applied to introduce a mutated ribosome binding site into the E. coli SJ_XTL219 genome and thereby increase dCas9sp expression by about 20-fold, creating a new strain SJ_XTL219-RBS1 (FIG. 10). The RT-qPCR measurements were then repeated on ELSA-Succinate in SJ_XTL219-RBS1 and all six genes were simultaneously knocked-down by 65 to 3552-fold (FIG. 7E).

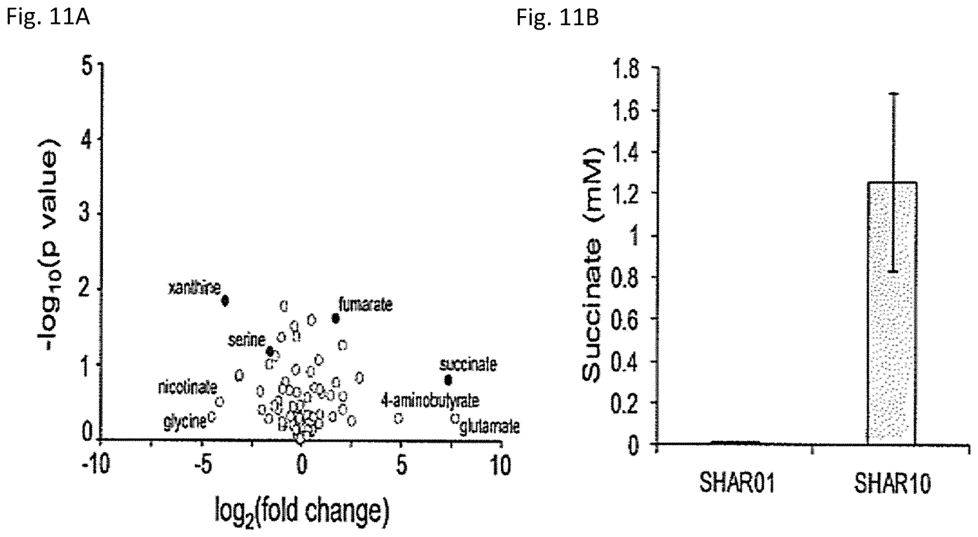

Metabolomics measurements were then applied to characterize how ELSA-Succinate affected cellular metabolite levels. First, the strain was adapted over a 3-day period in induced conditions, growing in M9 minimal media with glycerol with repeated serial dilutions, followed by confirmation of ELSA genomic integrity by sequencing. 24-hour cultures were then carried out in induced conditions using the same media, measuring metabolite levels in the culture supernatant using LC-MS3 in quantitation mode. Succinic acid titers increased by over 150-fold from about 0.008 to 1.25 mM. Intriguingly, several additional metabolites were found to have altered levels, including higher amounts of fumaric acid, glutamic acid, and 4-Aminobutyric acid as well as lower amounts of acetic acid, xanthine, glycine, serine, and niacin (FIG. 11). By simultaneously exerting control over several enzyme expression levels, a single ELSA could fundamentally rewire the cell's central metabolic flows.

In a second example, a second 15-sgRNA ELSA (ELSA-MultiAux) was designed to simultaneously knock-down the expression of 9 genes (hisD, proC, lysA, tyrA, aroF, pheA, leuA, ilvD, argH) (FIG. 8C), responsible for amino acid biosynthesis. Both RT-qPCR and RNA-Seq were carried out to measure the sgRNA expression levels and the targeted mRNAs expression levels. All 15 sgRNAs were consistently well-expressed, though interestingly, long transcripts were detected that contained multiple sgRNAs, likely due to incomplete transcriptional termination (FIG. 7D). When integrated into the dCas9 over-expression strain (SJ_XTL219-RBS1), ELSA-MultiAux simultaneously knocked down the expression of 7 genes by 1.6 to 233-fold (FIG. 7E).

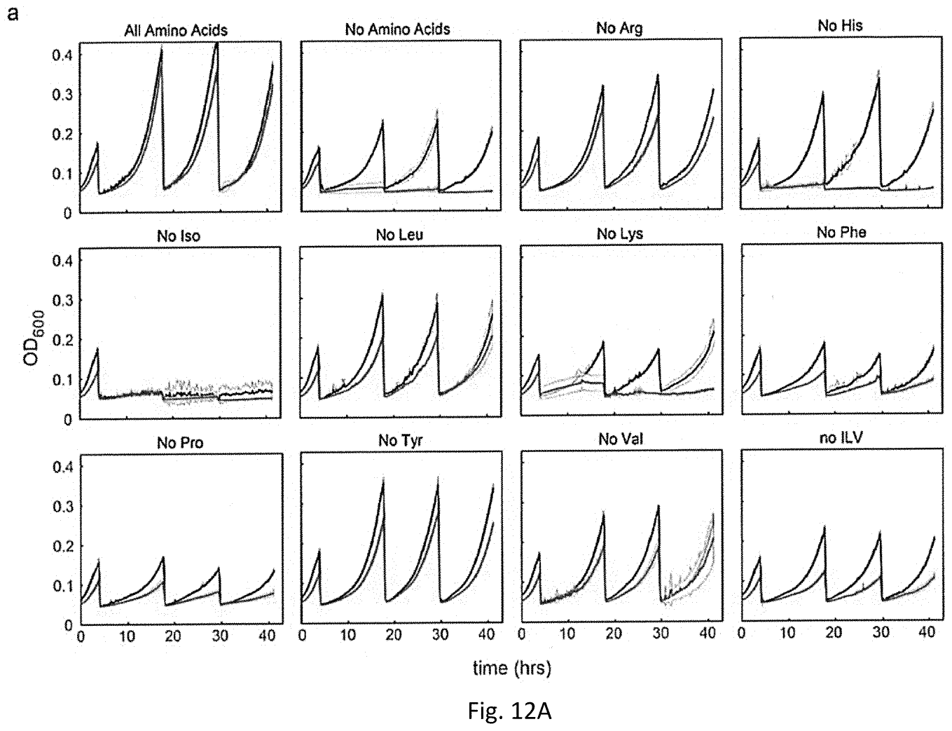

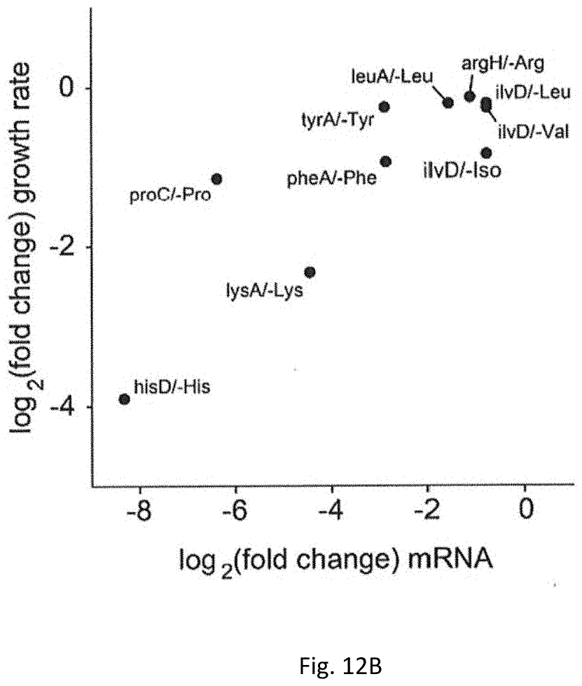

As expected, without an amino acid source, this strain had a highly selective bacteriostatic phenotype; after 18 hours in induced conditions, its growth rate dropped by 100-fold, compared to SJ_XTL219, and never recovered for a period of 44 hours (FIG. 12). Interestingly, when the strain was grown on media missing a single amino acid, there was a quantitative relationship between the strain's growth rate and the knock-down level of the enzyme responsible for the amino acid's biosynthesis (FIG. 12). There were also no detectable genomic mutations in ELSA-MultiAux after a 44-hour continuous culture in induced conditions, confirming the genetic stability of ELSAs under highly selective growth conditions.

Finally, in a third example, a 22-sgRNA ELSA (ELSA-Stress) was designed to simultaneously knock-down the expression of 13 genes (adiA, ansP, dgkA, ic1R, marR, mreC, narQ, plsB, wzb, ycfS, yncE, yncG, and yncH) (FIG. 8B), responsible for pH homeostasis, quorum sensing, stress response, and essential membrane biosynthesis. As before, both RT-qPCR and RNA-Seq were carried out to characterize its functional effects. All 22 sgRNAs were consistently well-expressed with previously observed amounts of incomplete transcriptional termination (FIG. 7D). When ELSA-Stress was integrated into the dCas9 over-expression strain (SJ_XTL219-RB S1), it simultaneously knocked down the expression of 9 genes by 3 to 162-fold (FIG. 7E).

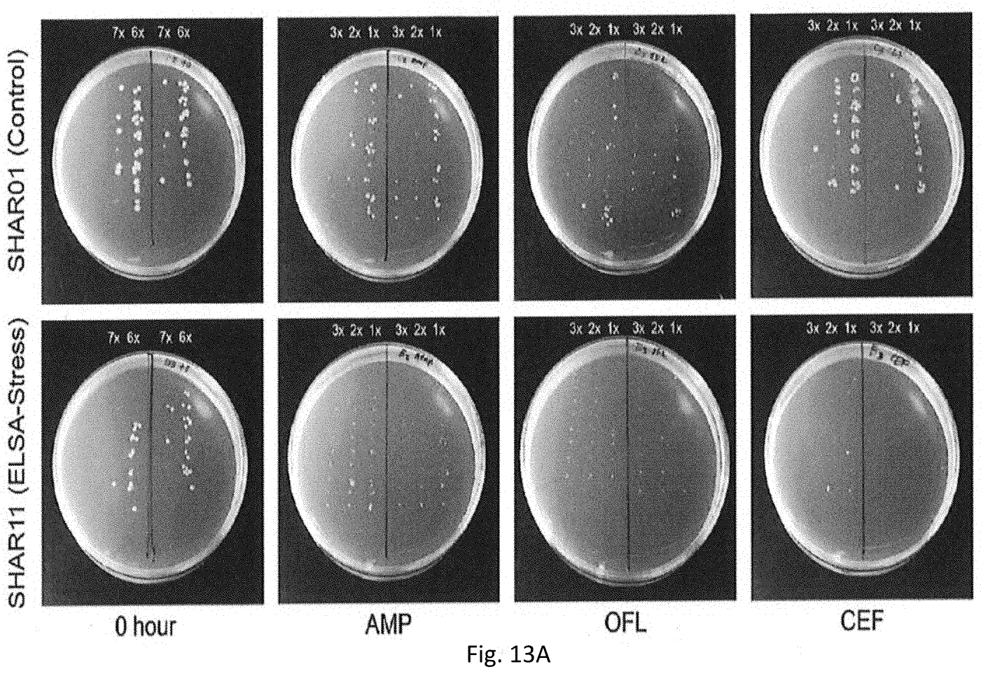

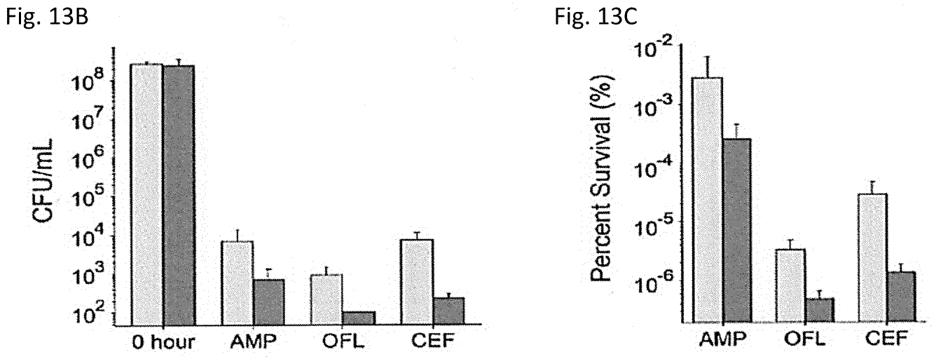

It was found that ELSA-Stress greatly inhibited the strain's ability to survive antibiotic treatment, reducing persister cell formation and survival. When stationary-phase cultures were treated with either 100 μg/mL ampicillin, 5 μg/mL ofloxacin, or 5 μg/mL cefixime, the strain expressing ELSA-Stress had a 11-fold, 7-fold, or 21-fold reduction in viable persister cells respectively, compared to a SJ_XTL219-RB S1 control strain (FIG. 13). Notably, there were no detectable genomic mutations in ELSA-Stress even after 50 hours of continuous culturing in induced conditions, again confirming the strain's genetic stability in a highly selective condition.

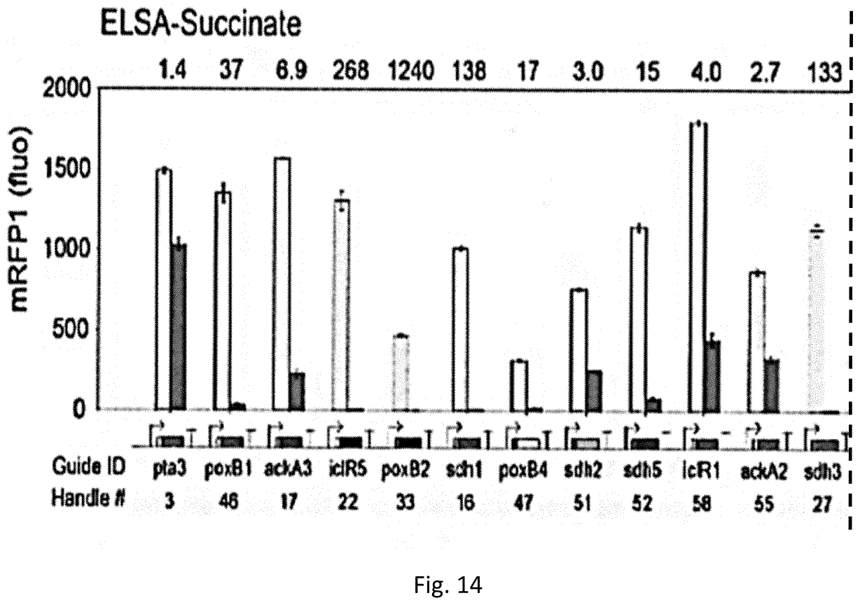

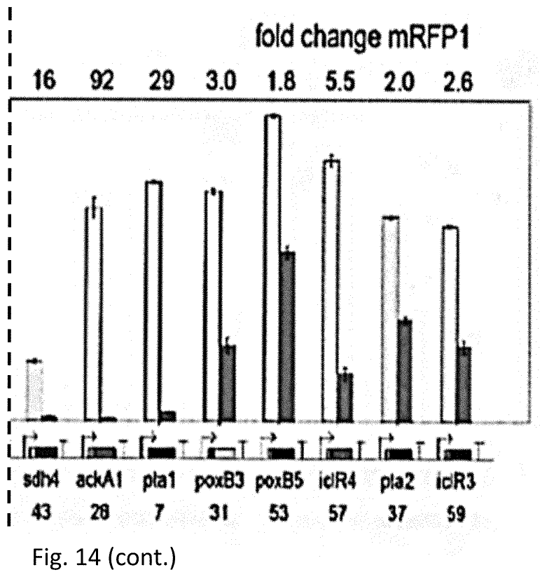

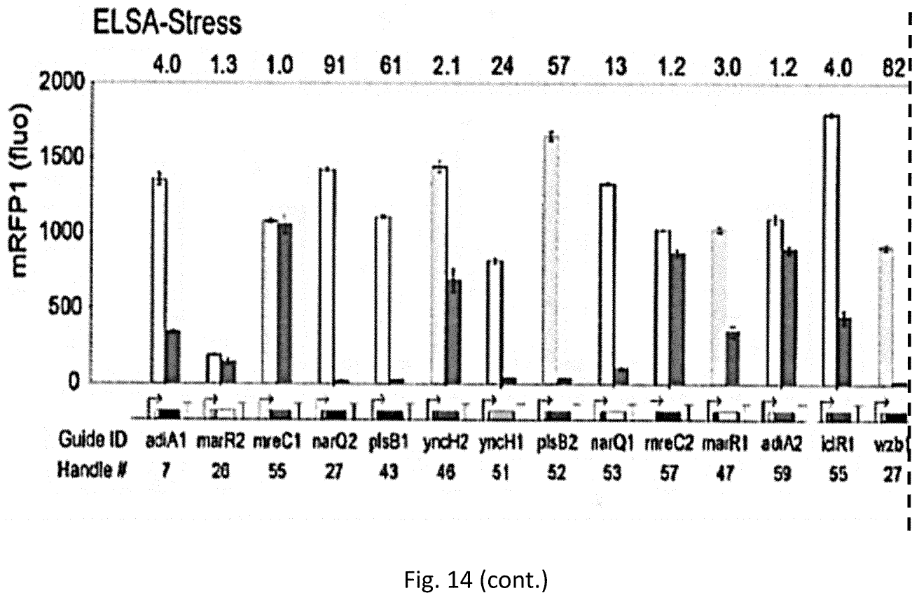

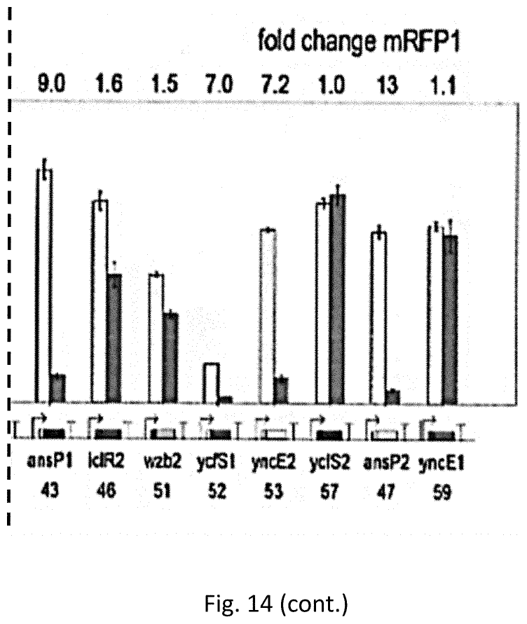

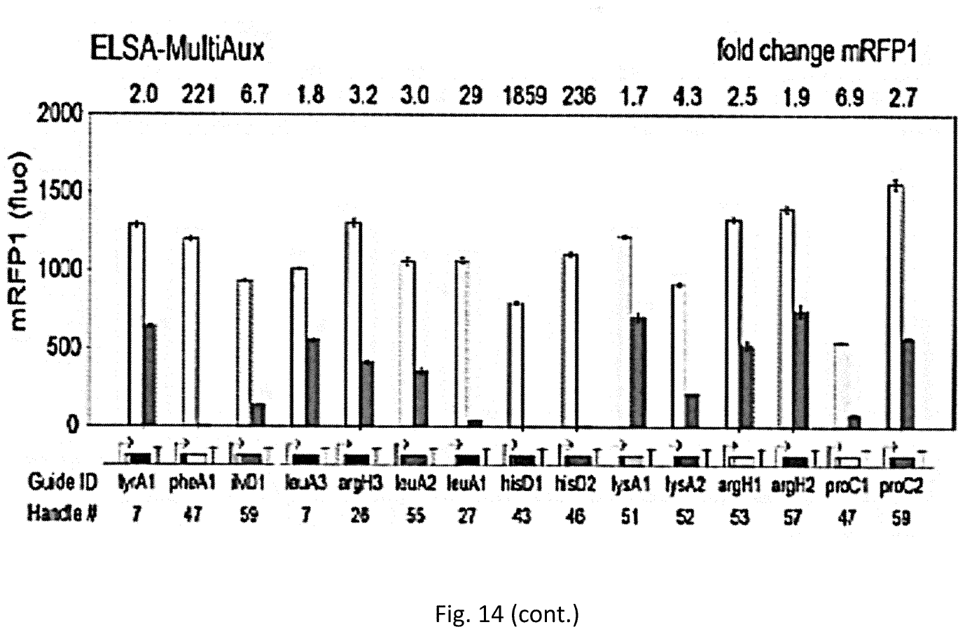

Overall, the ELSAs successfully repressed 85% of the targeted genes, binding to 57 distinct genomic sites and collectively utilizing 20 non-repetitive sgRNA handles. However, it was unclear how each sgRNA contributed to the gene-level knock-downs when co-expressed within an ELSA as most genes were targeted by 2 or 3 sgRNAs. Therefore, dCas9sp-mediated transcriptional repression levels were measured from all individual sgRNAs when they were co-expressed within their respective ELSAs. To do this, 57 reporter plasmids were constructed that utilize the corresponding sgRNA binding sites to regulate mRFP1 expression. They were transformed into the control E. coli SJ_XTL219-RBS1 strain and E. coli SJ_XTL219-RBS1 strains carrying either ELSA-Succinate, ELSA-MultiAux, or ELSA-Stress as genomic integrations. Overall, 81% of the individual sgRNAs were able to knock-down mRFP1 expression by at least 2-fold, though interestingly, sgRNAs using the same handle, but different guides, achieved greatly different knock-down levels (FIG. 14). For example, sgRNAs utilizing non-repetitive handle #46 knocked-down mRFP1 expression by either 236, 35, 2.1, or 1.6-fold, when using either the hisD2, poxB1, yncH2, or ic1R2 guide RNA sequences, respectively.

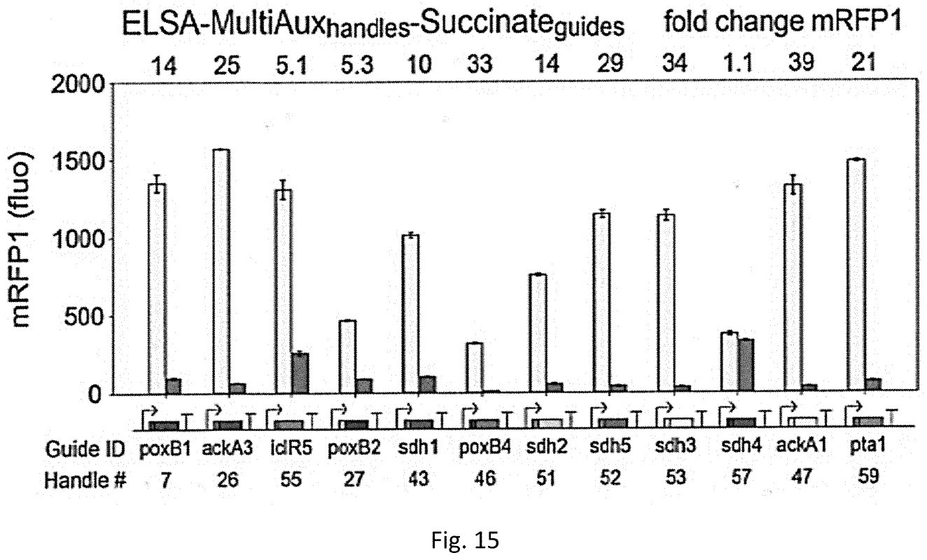

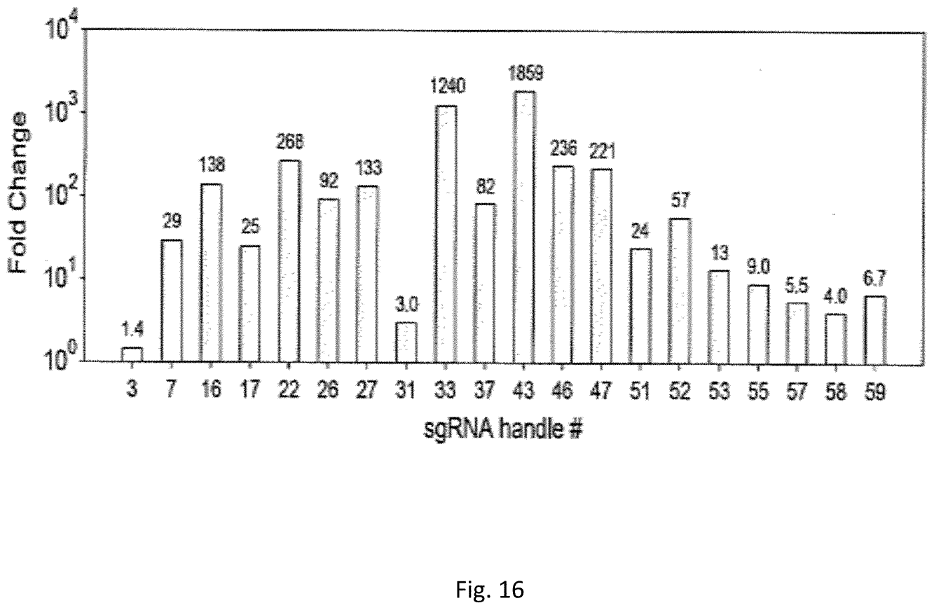

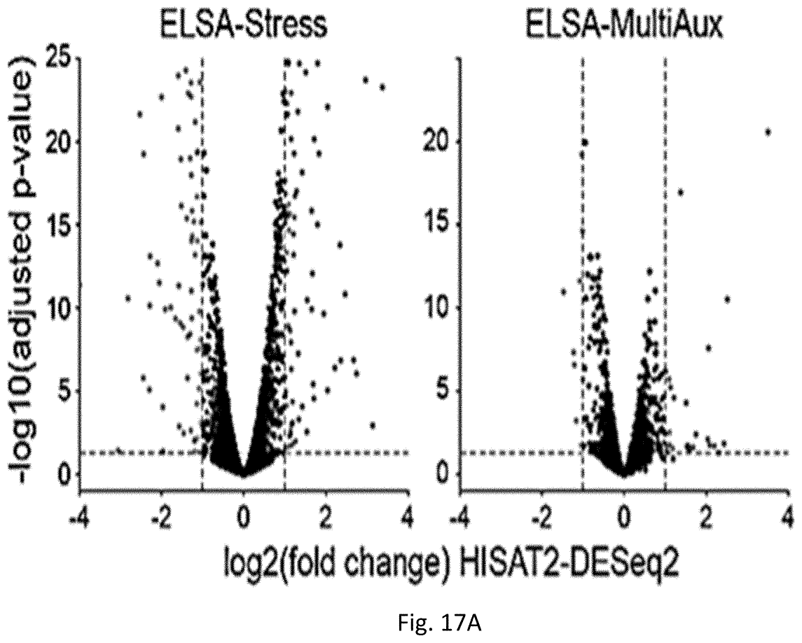

The potential for guide-handle interactions was intriguing, and therefore non-repetitive handles that exhibited such guide dependence were identified. The importance of guide-handle pairing was tested by constructing a new ELSA that combines these guide-dependent handles with guide RNAs from ELSA-Succinate, previously shown to support high knock-down levels, while scrambling sgRNA ordering within the ELSA to test position effects. Using reporter plasmids for characterization, it was found that 11 out of 12 sgRNAs knocked down mRFP1 expression by more than 5-fold, confirming the importance of guide RNA design on overall sgRNA activity (FIG. 15). Overall, across the 69 guide-handle pairings co-expressed within many-sgRNA ELSAs, 95% of the non-repetitive handles supported successful knock-downs (FIG. 16).

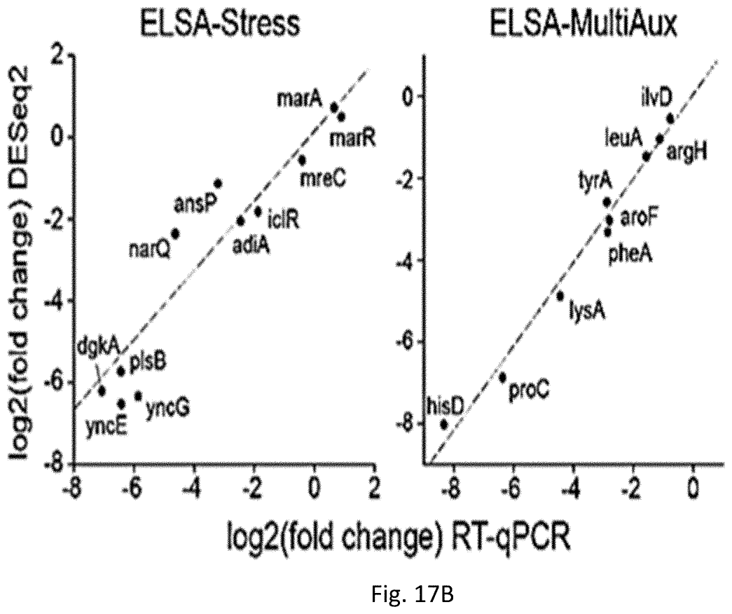

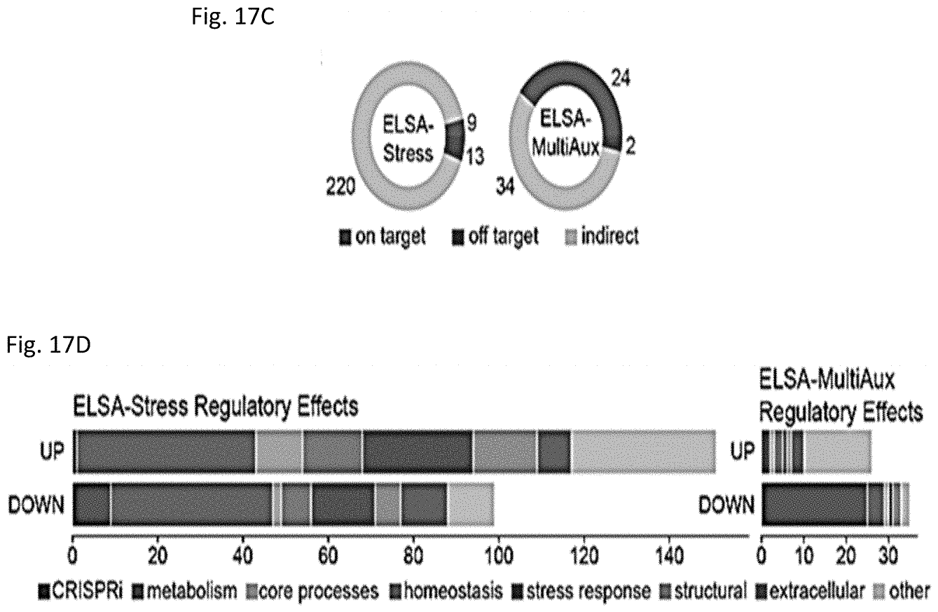

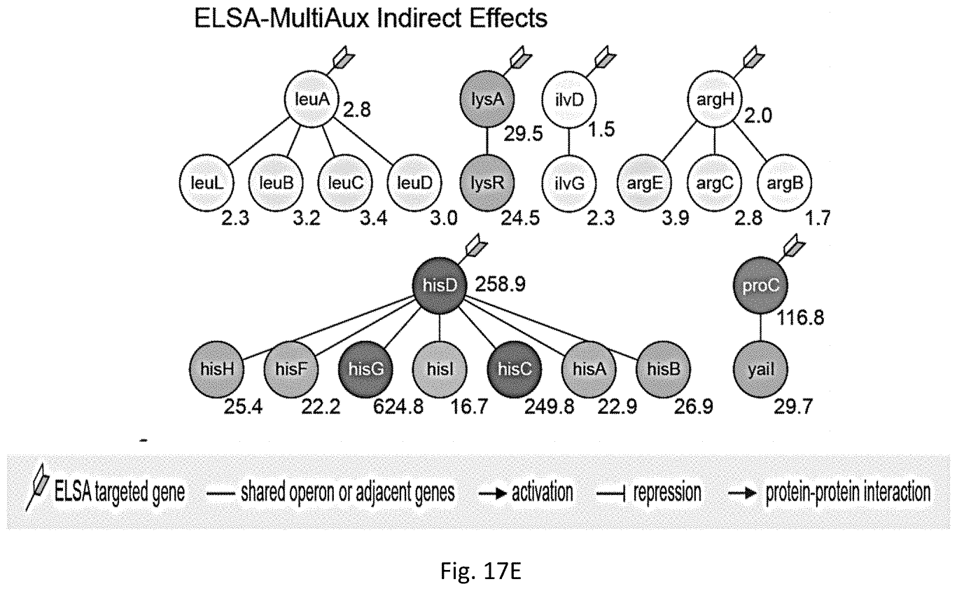

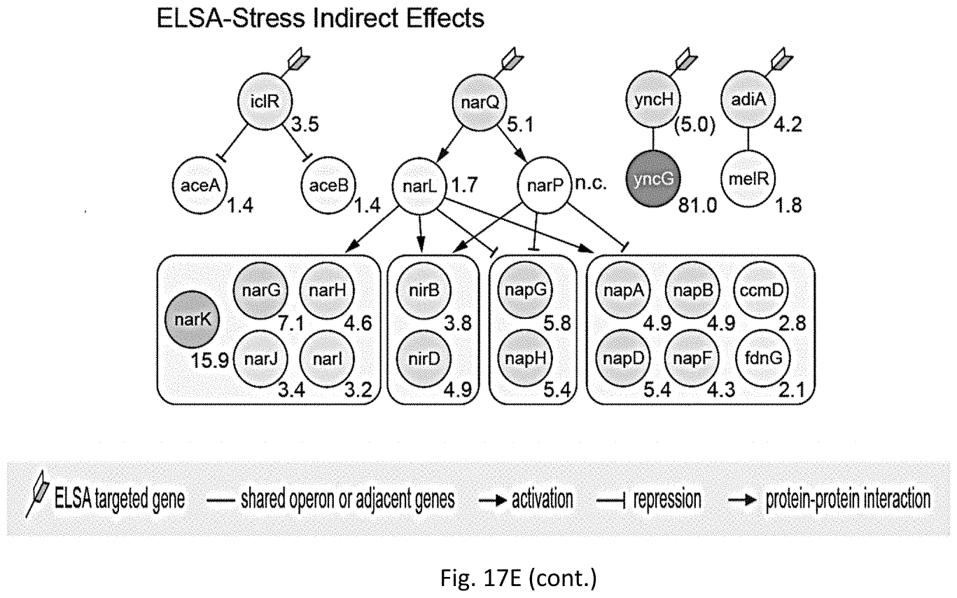

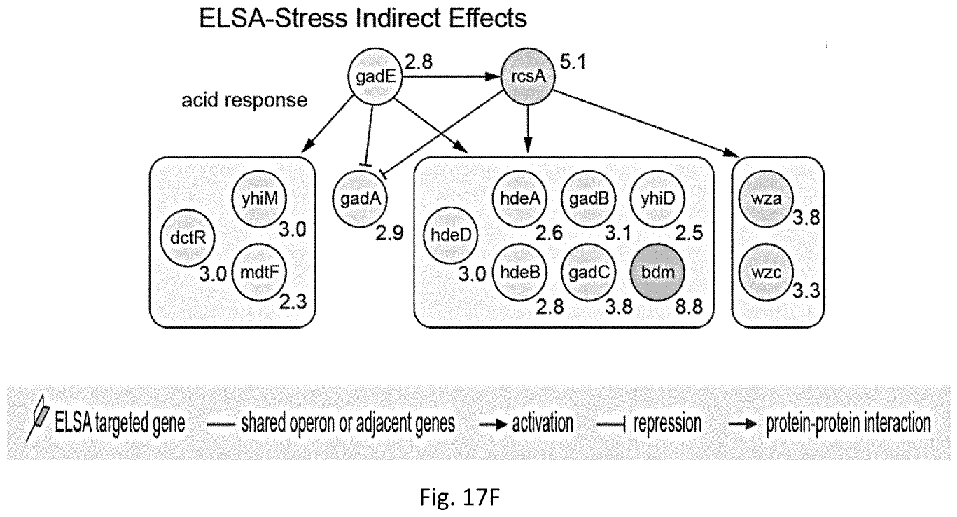

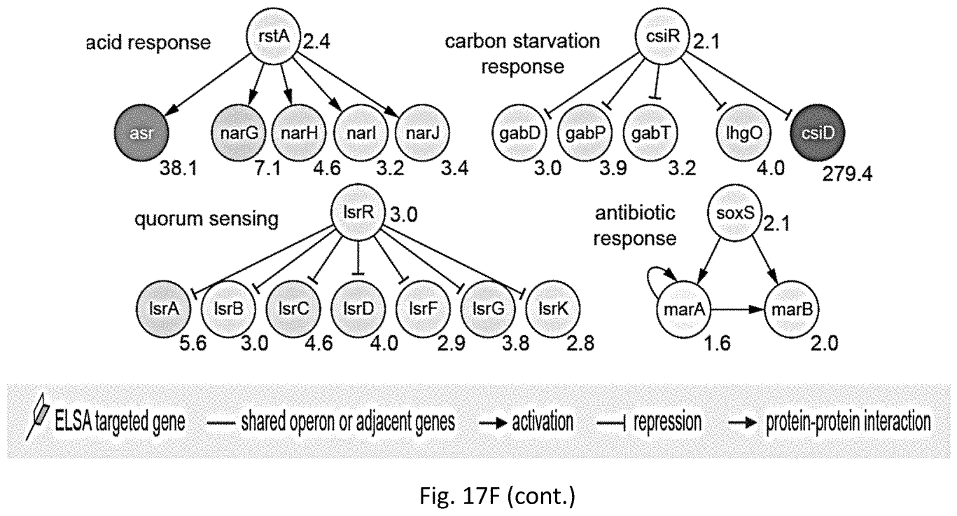

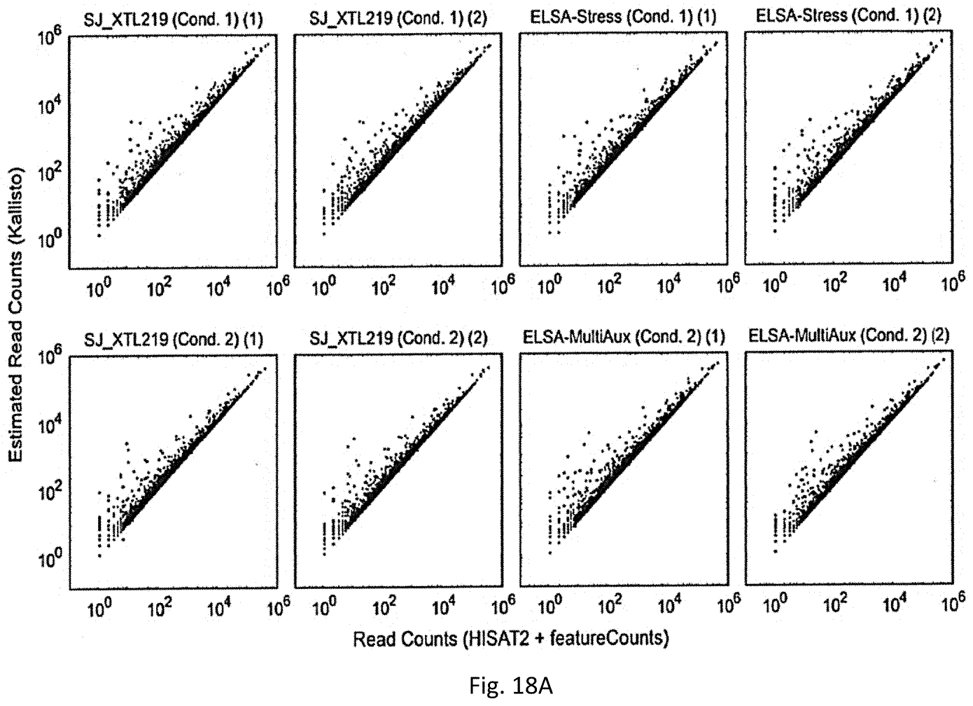

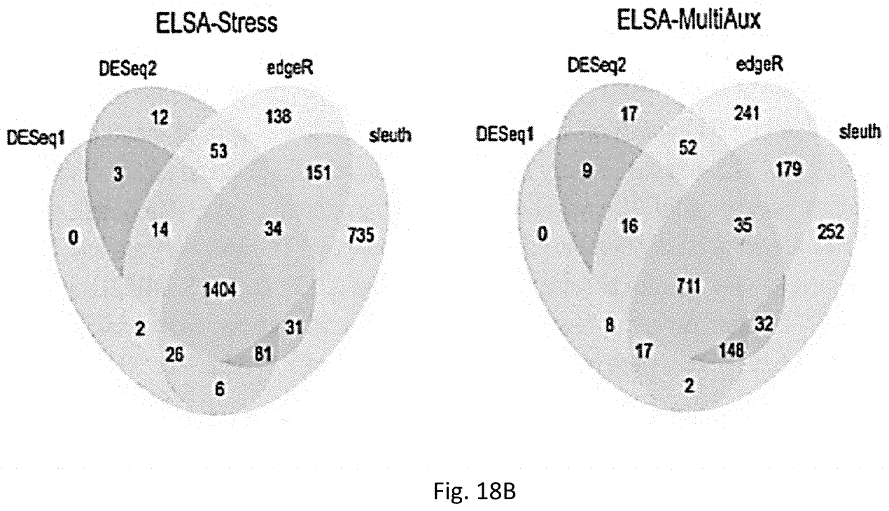

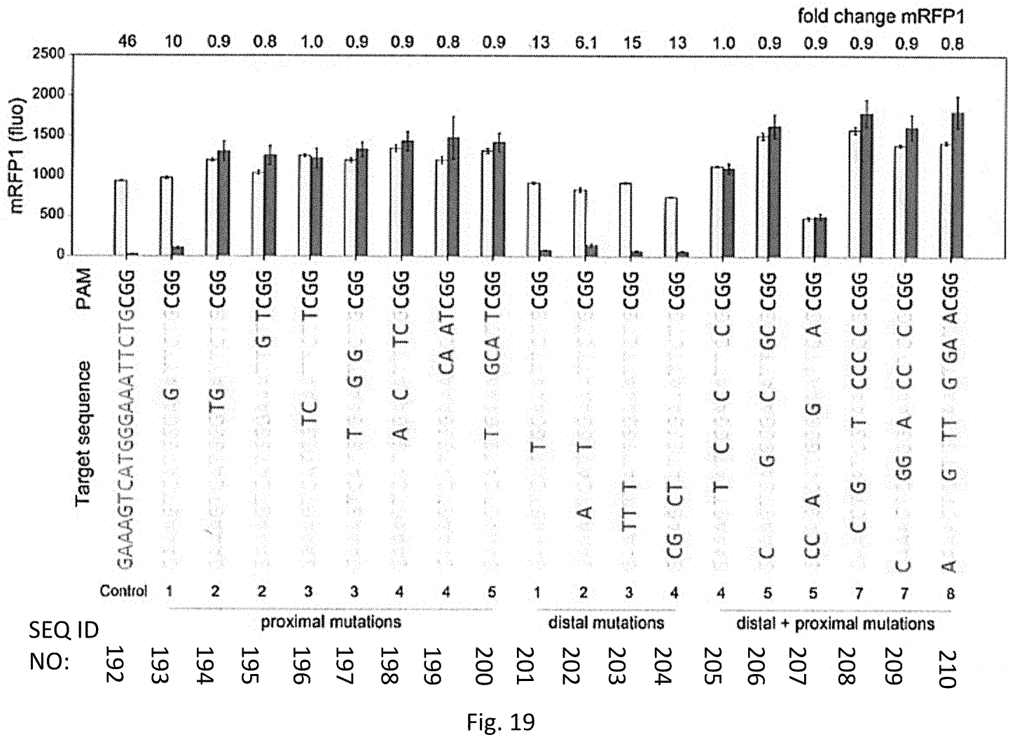

Next, the potential for off-target CRISPRi activity from the ELSAs was evaluated. RNA-Seq experiments were performed to measure how either ELSA-Stress or ELSA-MultiAux affected the transcriptome-wide mRNA levels of E. coli SJ_XTL219 in induced growth conditions. Differentially expressed genes were identified (FIG. 17A) using a consensus approach across two biological replicates and 4 RNA-Seq differential expression analysis pipelines (FIG. 18A and FIG. 18B). Both the RNA-Seq and RT-qPCR measurements yielded highly similar knock-down levels for the ELSAs' on-target genes (FIG. 17B).

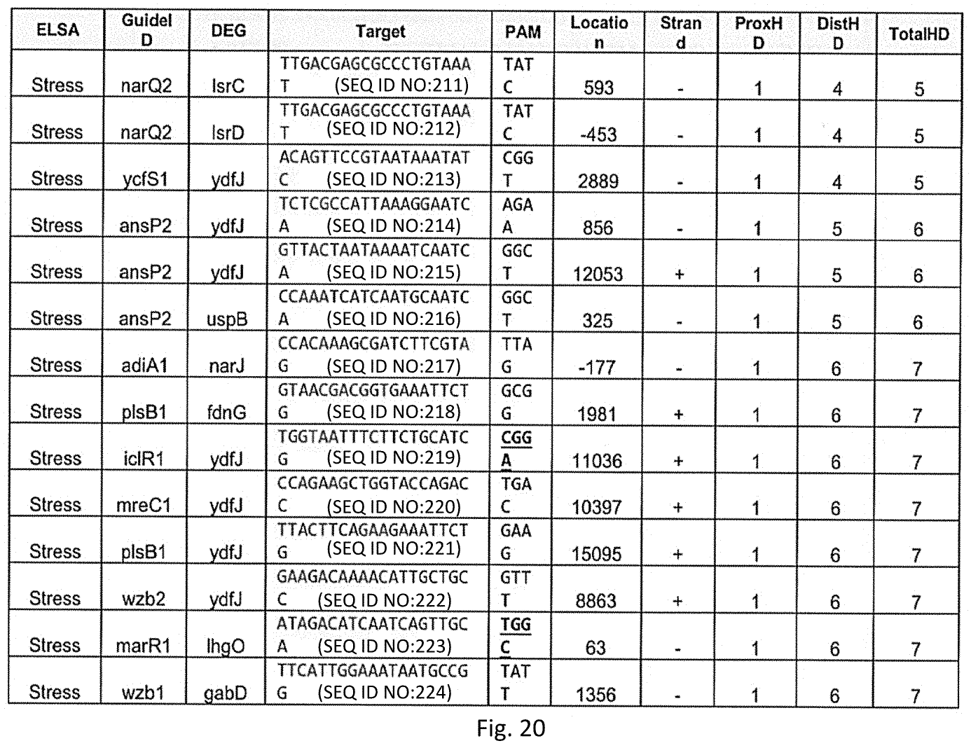

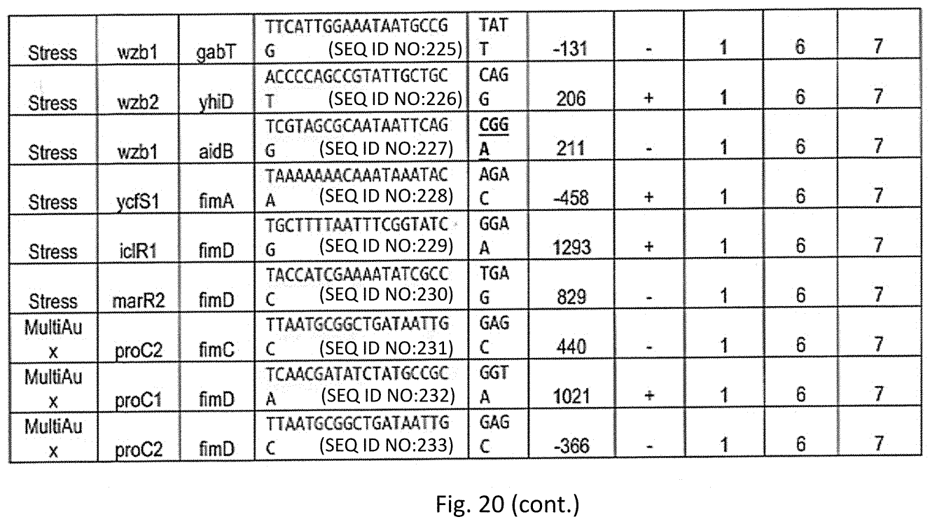

Surprisingly, the 22-sgRNA ELSA-Stress differentially regulated 242 genes of diverse function (FIG. 17C), including genes responsible for metabolism, stress response, and for producing structural proteins (FIG. 17D). With so many affected genes, without being bound by theory, it was speculated that many of them were affected through multiple layers of cascading regulatory interactions and necessarily not through off-target CRISPRi activity. The first step to testing this hypothesis was to determine how off-target sgRNA binding sites interacted with sgRNAs co-expressed within the non-repetitive ELSAs. To do this, 18 reporter plasmids were constructed containing off-target sgRNA binding sites with between 1 to 5 PAM-proximal mismatches, 1 to 4 PAM-distal mismatches, and mismatch combinations. As expected, it was found that introducing 2 or more PAM-proximal mismatches completely eliminated CRISPRi activity, while introducing PAM-distal mismatches had a more step-wise effect (FIG. 19), similar to previous models of guide RNA activity (Farasat et al., 2016, PLoS Computational Biology 12, e1004724; Doench et al., 2016, Nature biotechnology). These rules were then applied to examine the sequences surrounding the 242 differentially expressed genes, including regulatory regions. Only 13 repressed genes with candidate off-target sgRNA binding sites were found (FIG. 20).

In contrast, many indirect regulatory effects were identified that could explain how the remaining genes were differentially expressed (FIG. 17E). For example, ELSA-Stress directly repressed narQ, a two-component sensor kinase, by 5.1-fold, which led to a 1.7-fold repression of narL, a response regulator, that in turn activated 15 genes responsible for nitrate-dependent anaerobic respiration and electron transport. Gene regulatory cascades indirectly affected the cell's response to acid stress, carbon starvation, quorum sensing, and antibiotics (FIG. 17F). Gene regulatory feedback loops can also mitigate on-target CRISPRi activity. For example, ELSA-Stress successfully targeted the wzb site for knock-down, achieving reporter plasmid knock-down levels of 82-fold (FIG. 14), however the wzb endogenous mRNA levels actually increased by 2.5-fold (FIG. 7E). Wzb is a signaling protein, part of an activated kinase cascade, that is activated by the response regulators RcsA and RcsB. rcsA mRNA levels were activated by 5-fold (FIG. 17F), suggesting that transcriptional activation of wzb is confounding CRISPRi-mediated repression.

Similarly, the 15-sgRNA ELSA-MultiAux had a regulatory effect on 60 genes (FIG. 17C), but most of the down-regulated genes were either directly targeted by ELSA-MultiAux or located within the same operon as an on-target gene (FIG. 17D). For example, when ELSA-MultiAux knocked-down expression of hisD by 259-fold, genes within the same his operon were also repressed by 22 to 625-fold (FIG. 17E). Neighboring genes could also be similarly affected; for example, when ELSA-MultiAux repressed proC by 117-fold, yaiL was also repressed by 30-fold because their promoters share the same regions. Notably, only 2 repressed genes were identified with candidate off-target sgRNA binding sites (FIG. 20). Overall, these results show that the ELSAs significantly rewired transcriptome-wide mRNA levels, though mainly due to on-target CRISPRi activity and systems-level effects that depended on operon architecture as well as pre-existing signaling and gene regulatory networks.

Non-Repetitive Extra Long sgRNA Arrays

SEQ ID NO:119—ELSA-Succinate

SEQ ID NO:120—ELSA-MultiAux

SEQ ID NO:121—ELSA-MultiAux

SEQ ID NO:122—ELSA-stress

SEQ ID NO:123—ELSA-stress

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A nucleic acid molecule comprising an extra long sgRNA array (ELSA) for expression of at least two sgRNA sequences comprising:
nucleotide sequences encoding two or more sgRNA sequence, wherein each sgRNA encoding nucleotide sequence is under the control of a sgRNA promoter and operably linked to a sgRNA handle sequence; wherein the ELSA comprises a maximum shared repeat length of 20 nucleotides or less.

2. The composition of claim 1, wherein the ELSA comprises a maximum shared repeat length 12 nucleotides or less.

3. The composition of claim 1, wherein the ELSA comprises nucleotide sequences for expression of at least 5 sgRNAs.

4. The composition of claim 1, wherein the ELSA comprises at least two sgRNA promoter sequences selected from the group consisting of SEQ ID NO:1-64.

5. The composition of claim 1, wherein the ELSA comprises at least two sequences selected from the group consisting of SEQ ID NO:65-118.

6. A system comprising at least one ELSA of claim 1 and a RNA-guided enzyme or a nucleotide sequence encoding a RNA-guided enzyme.

7. The system of claim 6, wherein the ELSA comprises a maximum shared repeat length of 12 nucleotides or less.

8. The system of claim 6, wherein the ELSA comprises nucleotide sequences for expression of at least 5 sgRNA.

9. The system of claim 6, wherein the ELSA comprises at least two promoter sequences selected from the group consisting of SEQ ID NO:1-64.

10. The system of claim 6, wherein the ELSA comprises at least two sgRNA sequences selected from the group consisting of SEQ ID NO:65-118.

11. The system of claim 6, wherein the nucleotide sequence encoding a RNA-guided enzyme encodes an enzyme selected from the group consisting of a Cas9 enzyme and a catalytically dead Cas9.

12. A modified cell, wherein the cell comprises a system of claim 6.

13. A method of modulating the level or activity of one or more target gene comprising contacting a sample with the system of claim 6.

14. The method of claim 13, wherein the one or more target gene are associated with a biological pathway or process.

15. The method of claim 14, wherein the biological pathway or process is selected from the group consisting of cellular sugar catabolism, glycolysis, pentose phosphate pathway, pyruvate metabolism, citrate cycle, glyoxylate cycle, propanoate metabolism, butanoate metabolism, inositol phosphate metabolism, amino acid biosynthesis, nucleotide biosynthesis, fatty acid biosynthesis, terpenoid biosynthesis, steroid biosynthesis, glycan biosynthesis, riboflavin biosynthesis, thiamine biosynthesis, biotin biosynthesis, folate biosynthesis, retinol biosynthesis, polyketide biosynthesis, oxidative phosphorylation, methane metabolism, sulfur metabolism, nitrogen metabolism, photosynthesis, nitrogen fixation, carbon dioxide fixation, immune response, and the inflammatory response pathway.

16. The method of claim 13, wherein the one or more target gene are associated with a disease or disorder.

17. A method of treating a disease or disorder in a subject in need thereof, comprising administering to the subject a CRISPR/Cas9 system of claim 6, wherein the ELSA comprises nucleotide sequence for expression of two or more sgRNA specific for genes associated with the disease or disorder.

18. A nucleic acid molecule encoding an sgRNA, comprising a targeting sequence and an sgRNA handle sequence, wherein the sequence encoding the sgRNA handle comprises a variant of SEQ ID NO:65, comprising at least 80% identity to SEQ ID NO:65.

19. The nucleic acid molecule of claim 18, wherein the sequence encoding the sgRNA handle is selected from the group consisting of SEQ ID NO:66-SEQ ID NO:118.

20. An sgRNA encoded by the nucleic acid molecule of claim 18.

21. A nucleic acid molecule for expression of at least one sgRNA, comprising a promoter sequence selected from the group consisting of SEQ ID NO:1-64, or a variant or fragment thereof, operably linked to a sequence encoding an sgRNA.


SEQUENCE LISTING (233 sequences)

SEQ ID NO: 1 - DNA, 36 bp, Artificial Sequence
  Chemically Synthesized
tttataggttcactgtagaatcatacaatggactaa

SEQ ID NO: 2 - DNA, 36 bp, Artificial Sequence
  Chemically Synthesized
tttatgagagtattcctccgatttacaatgagacta

SEQ ID NO: 3 - DNA, 36 bp, Artificial Sequence
  Chemically Synthesized
tttatacggttcttacgaaataatacaatggcttta

SEQ ID NO: 4 - DNA, 36 bp, Artificial Sequence
  Chemically Synthesized
tttatagactccagtagtgtggatacaatgctagcg

SEQ ID NO: 5 - DNA, 36 bp, Artificial Sequence
  Chemically Synthesized
ttgacatgttcccaataagagcagactatgcttagc

SEQ ID NO: 6 - DNA, 36 bp, Artificial Sequence
  Chemically Synthesized
tttatgggacggtttatcaatactacaatgcttagc

SEQ ID NO: 7 - DNA, 36 bp, Artificial Sequence
  Chemically Synthesized
tttataactttactacagggagatacaatgactagc

SEQ ID NO: 8 - DNA, 36 bp, Artificial Sequence
  Chemically Synthesized
ttgacaacaatctgtagcagttcgactatgctctag

SEQ ID NO: 9 - DNA, 36 bp, Artificial Sequence
  Chemically Synthesized
tttatacaataagttcgttgtcgtacaatgatcata

SEQ ID NO: 10 - DNA, 36 bp, Artificial Sequence
  Chemically Synthesized
tttatatgacttaccactattggtacaatggcctag

SEQ ID NO: 11 - DNA, 36 bp, Artificial Sequence
  Chemically Synthesized
tttatggattttaccaaccgaggtacaatgccctaa

SEQ ID NO: 12 - DNA, 36 bp, Artificial Sequence
  Chemically Synthesized
tttatgactcgtagcgttcagtatacaatgcctgag

SEQ ID NO: 13 - DNA, 36 bp, Artificial Sequence
  Chemically Synthesized
ttgacaaagagattttcactcgggactatgctaggg

SEQ ID NO: 14 - DNA, 36 bp, Artificial Sequence
  Chemically Synthesized
tttatgttgaatagtatccacgctacaatgcggata

SEQ ID NO: 15 - DNA, 36 bp, Artificial Sequence
  Chemically Synthesized
tttatatcgtcacactgaagagttacaatgtctcag

SEQ ID NO: 16 - DNA, 36 bp, Artificial Sequence
  Chemically Synthesized
ttgacagggcaataaatcgttacgactatgtctagc

SEQ ID NO: 17 - DNA, 36 bp, Artificial Sequence
  Chemically Synthesized
tttatatagatagcagattgacctacaatgcatgta

SEQ ID NO: 18 - DNA, 36 bp, Artificial Sequence
  Chemically Synthesized
ttgacatgcgttgaaacagtaacgactatgcaatag

SEQ ID NO: 19 - DNA, 36 bp, Artificial Sequence
  Chemically Synthesized
ttgacacctgtgagattcatagagactatgtcctta

SEQ ID NO: 20 - DNA, 36 bp, Artificial Sequence
  Chemically Synthesized
tttatgcgactgataacctgttgtacaatgctcagc

SEQ ID NO: 21 - DNA, 36 bp, Artificial Sequence
  Chemically Synthesized
tttatactcaatacggtgtctgatacaatgtcgtag

SEQ ID NO: 22 - DNA, 36 bp, Artificial Sequence
  Chemically Synthesized
tttatgccacgataagtgttacttacaatgctgcta

SEQ ID NO: 23 - DNA, 36 bp, Artificial Sequence
  Chemically Synthesized
ttgacagagtcagaaactttaccgactatgatctag

SEQ ID NO: 24 - DNA, 36 bp, Artificial Sequence
  Chemically Synthesized
ttgacagactcgcagtttcaatagactatgcctagc

SEQ ID NO: 25 - DNA, 36 bp, Artificial Sequence
  Chemically Synthesized
ttgacatattacaactctgctgagactatgcgtagc

SEQ ID NO: 26 - DNA, 35 bp, Artificial Sequence
  Chemically Synthesized
tttatgaagttctctgaaacagatacaatgctagc

SEQ ID NO: 27 - DNA, 35 bp, Artificial Sequence
  Chemically Synthesized
tttatattcagactcggtataggtacaatgctagc

SEQ ID NO: 28 - DNA, 35 bp, Artificial Sequence
  Chemically Synthesized
ttgacactgtaactgcgaatagagactatgctagc

SEQ ID NO: 29 - DNA, 35 bp, Artificial Sequence
  Chemically Synthesized
tttacgccgtgaagtaatacagatactatgctagc

SEQ ID NO: 30 - DNA, 35 bp, Artificial Sequence
  Chemically Synthesized
tttacgaaggaactgtctataggtacaatgctagc

SEQ ID NO: 31 - DNA, 35 bp, Artificial Sequence
  Chemically Synthesized
tttatgactttcgtaggcatagatacaatgctagc

SEQ ID NO: 32 - DNA, 35 bp, Artificial Sequence
  Chemically Synthesized
tttataagcaacttcggtataggtacaatgctagc

SEQ ID NO: 33 - DNA, 35 bp, Artificial Sequence
  Chemically Synthesized
ttgacatggctgtatcacatagggactatgctagc

SEQ ID NO: 34 - DNA, 35 bp, Artificial Sequence
  Chemically Synthesized
tttacgtcgttatcagcgaccgatactatgctagc

SEQ ID NO: 35 - DNA, 35 bp, Artificial Sequence
  Chemically Synthesized
tttacgtactggtgaactataggtacaatgctagc

SEQ ID NO: 36 - DNA, 35 bp, Artificial Sequence
  Chemically Synthesized
ttgacatgactctccagctgtgctataattgtact

SEQ ID NO: 37 - DNA, 35 bp, Artificial Sequence
  Chemically Synthesized
ttgacatttcgtcaagagtcgactataatatcgcg

SEQ ID NO: 38 - DNA, 34 bp, Artificial Sequence
  Chemically Synthesized
ttgacatgagctcgtcgtcaggatatatagcttt

SEQ ID NO: 39 - DNA, 35 bp, Artificial Sequence
  Chemically Synthesized
ttgacatgaagtgttagacgtcatataatcgtggt

SEQ ID NO: 40 - DNA, 35 bp, Artificial Sequence
  Chemically Synthesized
ttgacataggcaagccagtatagtataatcacata

SEQ ID NO: 41 - DNA, 35 bp, Artificial Sequence
  Chemically Synthesized
ttgacagtcctcgaacacctctatataatagtgtc

SEQ ID NO: 42 - DNA, 35 bp, Artificial Sequence
  Chemically Synthesized
ttgacagtagatcagagggttgctataatcgacag

SEQ ID NO: 43 - DNA, 35 bp, Artificial Sequence
  Chemically Synthesized
ttgacactaccgagacagtgacatataataggacc

SEQ ID NO: 44 - DNA, 35 bp, Artificial Sequence
  Chemically Synthesized
ttgacacgatgcttgctgctacctataataacata

SEQ ID NO: 45 - DNA, 35 bp, Artificial Sequence
  Chemically Synthesized
ttgacaactgctcagcgaaatactataatgactac

SEQ ID NO: 46 - DNA, 35 bp, Artificial Sequence
  Chemically Synthesized
ttgacaggtgaacgctcagctcttataatgcctat

SEQ ID NO: 47 - DNA, 35 bp, Artificial Sequence
  Chemically Synthesized
ttgacactggcctgacaagtccatataatgatgtc

SEQ ID NO: 48 - DNA, 35 bp, Artificial Sequence
  Chemically Synthesized
ttgacactatggtccgcaagcattataatgctctg

SEQ ID NO: 49 - DNA, 35 bp, Artificial Sequence
  Chemically Synthesized
ttgacaaagtactactgtattagtataattgtcat

SEQ ID NO: 50 - DNA, 35 bp, Artificial Sequence
  Chemically Synthesized
ttgacatgcgtgatttaacattctataattgcaca

SEQ ID NO: 51 - DNA, 35 bp, Artificial Sequence
  Chemically Synthesized
ttgacataagtcgtattcaaagatataatataggt

SEQ ID NO: 52 - DNA, 35 bp, Artificial Sequence
  Chemically Synthesized
ttgacagttgtgttatccggccatataatatctct

SEQ ID NO: 53 - DNA, 35 bp, Artificial Sequence
  Chemically Synthesized
ttgacagtgtgctaaaatttgtctataatgagtac

SEQ ID NO: 54 - DNA, 35 bp, Artificial Sequence
  Chemically Synthesized
ttgacagcatctgctttgtcacctataattcaatg

SEQ ID NO: 55 - DNA, 35 bp, Artificial Sequence
  Chemically Synthesized
ttgacagaccttatctacatggttataatctgaat

SEQ ID NO: 56 - DNA, 35 bp, Artificial Sequence
  Chemically Synthesized
ttgacactttgcacatgtcccgttataatcatgat

SEQ ID NO: 57 - DNA, 35 bp, Artificial Sequence
  Chemically Synthesized
ttgacacggatcttcgctgaacgtataatgagaaa

SEQ ID NO: 58 - DNA, 34 bp, Artificial Sequence
  Chemically Synthesized
ttgacacagcccagccggagagtataatcctatt

SEQ ID NO: 59 - DNA, 35 bp, Artificial Sequence
  Chemically Synthesized
ttgacaatcgctgtctacgtgaatataatgaattt

SEQ ID NO: 60 - DNA, 35 bp, Artificial Sequence
  Chemically Synthesized
ttgacattagcacttgagctgattataatgggccg

SEQ ID NO: 61 - DNA, 35 bp, Artificial Sequence
  Chemically Synthesized
ttgacagaggcagtactaccgtttataattcggac

SEQ ID NO: 62 - DNA, 35 bp, Artificial Sequence
  Chemically Synthesized
ttgacacctcatcttatagttcctataatttctat

SEQ ID NO: 63 - DNA, 35 bp, Artificial Sequence
  Chemically Synthesized
ttgacaccgggttgaatactatctataatgtacgg

SEQ ID NO: 64 - DNA, 35 bp, Artificial Sequence
  Chemically Synthesized
ttgacacattaggatggacgtattataatatgccc

SEQ ID NO: 65 - DNA, 61 bp, Artificial Sequence
  Chemically Synthesized
gttttagagctagaaatagcaagttaaaataaggctagtccgttatcaacttgaaaaagtg

SEQ ID NO: 66 - DNA, 61 bp, Artificial Sequence
  Chemically Synthesized
gttctagagctcgaaagagcaacttagaataagcctaatccctgatcaacttgaaaaagtc

SEQ ID NO: 67 - DNA, 61 bp, Artificial Sequence
  Chemically Synthesized
gttctagagctggtaacagcaagttagaataagtctagtccattatcaactggaaacagtg

SEQ ID NO: 68 - DNA, 61 bp, Artificial Sequence
  Chemically Synthesized
gtttttgagcgagaaatcgcaagtaaaaataaggctcgtccgttaacaagttgaaaaactg

SEQ ID NO: 69 - DNA, 61 bp, Artificial Sequence
  Chemically Synthesized
gttttatagctagaaatagcaagataaaataaggctagtccattatcaacttgaaaaagtg

SEQ ID NO: 70 - DNA, 61 bp, Artificial Sequence
  Chemically Synthesized
gttttgcagctagaaatagcaaggcaaaataatgctagtccgttcccaacttgaaaaagtg

SEQ ID NO: 71 - DNA, 61 bp, Artificial Sequence
  Chemically Synthesized
gttttagatcacgaaagtgaaagttaaaataagcctagcccgttaccaactggaaacagtg

SEQ ID NO: 72 - DNA, 61 bp, Artificial Sequence
  Chemically Synthesized
gttttggagctagaaatagcaagtcaaaataaggctagtccgttctcaacttgaaaaagtg

SEQ ID NO: 73 - DNA, 61 bp, Artificial Sequence
  Chemically Synthesized
gttttagagatggaaacatcaagttaaaataaggcaagtccgttaacaactcgaaagagtg

SEQ ID NO: 74 - DNA, 61 bp, Artificial Sequence
  Chemically Synthesized
gtgttagagttggaaacaacaagttaacataaggctactcggatttcaacgtgaaaacgtc

SEQ ID NO: 75 - DNA, 61 bp, Artificial Sequence
  Chemically Synthesized
gttttagagctagcaatagcaagctaaaataatgctagtccgttattaacttgaaaaagtg

SEQ ID NO: 76 - DNA, 61 bp, Artificial Sequence
  Chemically Synthesized
ggtttagagttagaaataacaagttaaactaaggctagtccgttataaacttgaaaaagtc

SEQ ID NO: 77 - DNA, 61 bp, Artificial Sequence
  Chemically Synthesized
gttttagagcttgaaaaagcaagttaaaattaggctagtccgttaacaacttgaaaaagtg

SEQ ID NO: 78 - DNA, 61 bp, Artificial Sequence
  Chemically Synthesized
gtattagagctagaaatagcaacttaatataaggctagtcggttatcaccttgaaaaaggg

SEQ ID NO: 79 - DNA, 61 bp, Artificial Sequence
  Chemically Synthesized
gttttcgagctagtaatagcaagtgaaaatgaagttagtccgttagcaaactgaaaagtta

SEQ ID NO: 80 - DNA, 61 bp, Artificial Sequence
  Chemically Synthesized
gttgtagatctagaaatagaatgttacaattaggctagtccgttatgaacatgaaaatgtg

SEQ ID NO: 81 - DNA, 61 bp, Artificial Sequence
  Chemically Synthesized
gtttgagagatcgaaagatcaagttcaaacaagtctagtccgttgtgaacctgaaaaggtg

SEQ ID NO: 82 - DNA, 61 bp, Artificial Sequence
  Chemically Synthesized
gttttagagctacacatagcaagttaaaataaaggtagtccgttatcagtttgaaaaaacg

SEQ ID NO: 83 - DNA, 61 bp, Artificial Sequence
  Chemically Synthesized
gttgtagagctagaaatagcgagttacaataaggctagtccgttatgaacttgaaaaagtg

SEQ ID NO: 84 - DNA, 61 bp, Artificial Sequence
  Chemically Synthesized
gttttagagtgagaaatcacaagttaaaataaggctagaccgttatcaactagaaatagtg

SEQ ID NO: 85 - DNA, 61 bp, Artificial Sequence
  Chemically Synthesized
gtttaagggttagaaataacaagtttaaataaggcaagtccgttatcaagtggcaacactc

SEQ ID NO: 86 - DNA, 61 bp, Artificial Sequence
  Chemically Synthesized
gctttagaccttgaaaaaggaagttaaagtaaggctagtccgttatgaccttgaaaaaggg

SEQ ID NO: 87 - DNA, 61 bp, Artificial Sequence
  Chemically Synthesized
gttttacacctagaaataggaaggtaaaataaggctggtccgttatcacctcgaaagaggg

SEQ ID NO: 88 - DNA, 61 bp, Artificial Sequence
  Chemically Synthesized
gttgtagagctagcaatagcaggttacaataaggctcgtccgttataaacatgaaaatgtg

SEQ ID NO: 89 - DNA, 61 bp, Artificial Sequence
  Chemically Synthesized
gatttcgagctaggcatagcaagtgaaattaaggctggtccattaacaccttgaaaaaggg

SEQ ID NO: 90 - DNA, 61 bp, Artificial Sequence
  Chemically Synthesized
gctttacagctagaaatagcagggtaaagtaaggctagtccgtaataaacgtgaaaacgtg

SEQ ID NO: 91 - DNA, 61 bp, Artificial Sequence
  Chemically Synthesized
gtttcagagcaagaaattgcaagttgaaataaggctagtccgttaaaaacttgaaaaagtg

SEQ ID NO: 92 - DNA, 61 bp, Artificial Sequence
  Chemically Synthesized
gtatctgaactcgacagagtaagtagatataaggccagtccgttagcaacttgaaaaagtc

SEQ ID NO: 93 - DNA, 61 bp, Artificial Sequence
  Chemically Synthesized
gttttagacctagaaataggaagttaaaataaggctagttcgttatcatcttgaaaaagag

SEQ ID NO: 94 - DNA, 61 bp, Artificial Sequence
  Chemically Synthesized
cttttagagatagaaatatcaagttaaaagaaggctagtccgttaccaacttgaaaaagtg

SEQ ID NO: 95 - DNA, 61 bp, Artificial Sequence
  Chemically Synthesized
gatttagagctggaaacagcaagttaaattaaggctagtccgttatcagcttgaaaaagcg

SEQ ID NO: 96 - DNA, 61 bp, Artificial Sequence
  Chemically Synthesized
gttgtagaggaagaaattccaagttacaatgaggctagtccgtgatgaacttgaaaaagtg

SEQ ID NO: 97 - DNA, 61 bp, Artificial Sequence
  Chemically Synthesized
gctttatatctagaaatagagagataaagtaaggcaagtccgttatcatctggaaacagac

SEQ ID NO: 98 - DNA, 61 bp, Artificial Sequence
  Chemically Synthesized
gtttaagagctagaaatagcacgtttaaataaggctagtccgttttcaacttgaaaaagtg

SEQ ID NO: 99 - DNA, 61 bp, Artificial Sequence
  Chemically Synthesized
gttttacagctagtgatagcaaggtaaaataaggctagtcccaaatcaacttgaaaaagtg

SEQ ID NO: 100 - DNA, 61 bp, Artificial Sequence
  Chemically Synthesized
gctttagagctagaaatagcaggttaaagtaaggccagtccgtaataaactggaaacagtg

SEQ ID NO: 101 - DNA, 61 bp, Artificial Sequence
  Chemically Synthesized
gtgttagagtcagatatgacatgttaacattaggctagtccggggtgaagttgaaaaactg

SEQ ID NO: 102 - DNA, 61 bp, Artificial Sequence
  Chemically Synthesized
ttactagagtgacaaatcacaagttagtaaaaggctagaccgttataatcccgaacgggag

SEQ ID NO: 103 - DNA, 61 bp, Artificial Sequence
  Chemically Synthesized
ttttcagatttggaaacaaaacgttgaaaaaaggcaagtccgttatgaacgcgaaagcgtg

SEQ ID NO: 104 - DNA, 61 bp, Artificial Sequence
  Chemically Synthesized
gtatgcgaggtagaaatacccagtgcatatcaggctagtccgatatcatgttgaagaacag

SEQ ID NO: 105 - DNA, 61 bp, Artificial Sequence
  Chemically Synthesized
cggttaggataagaaattataagttaaccgtaggctagcccgttataaactggaaacagtg

SEQ ID NO: 106 - DNA, 61 bp, Artificial Sequence
  Chemically Synthesized
gatgtagatgtagaaatacaaggttacattaaggcccgtccgtaatcaacttgaagaagtg

SEQ ID NO: 107 - DNA, 61 bp, Artificial Sequence
  Chemically Synthesized
gttttggacctagaaataggaagtcaaaataaggctggaccgacatgtaatcgaaagattt

SEQ ID NO: 108 - DNA, 61 bp, Artificial Sequence
  Chemically Synthesized
ccttaagagctagcaatagcaagtttaaggaaggcaagcccgttatcatcctgaataggac

SEQ ID NO: 109 - DNA, 61 bp, Artificial Sequence
  Chemically Synthesized
gtaatagagatggatacatcaagttattataaggctcgaccgttaacagtctgaaaagacg

SEQ ID NO: 110 - DNA, 61 bp, Artificial Sequence
  Chemically Synthesized
gttggagagcaagacattgcaagttccaataaggcgtgtccgataaaagcttgagaaagca

SEQ ID NO: 111 - DNA, 61 bp, Artificial Sequence
  Chemically Synthesized
atctgagagccaaaaatggcaagttcagataaggccagaccgttaccagcttaaataagcg

SEQ ID NO: 112 - DNA, 61 bp, Artificial Sequence
  Chemically Synthesized
gcttcagatccagaaatggaaagttgaagtgaggcaggtccggtagcaactcgaaagagtg

SEQ ID NO: 113 - DNA, 61 bp, Artificial Sequence
  Chemically Synthesized
agtttagagaatgcaaattcaagttaaactaaggcgagtccggtataatcgtgtaaacgag

SEQ ID NO: 114 - DNA, 61 bp, Artificial Sequence
  Chemically Synthesized
ggaatagaaaacaaaagtttaagttattctaaggccagtccggaatcatcctaaaaaggag

SEQ ID NO: 115 - DNA, 61 bp, Artificial Sequence
  Chemically Synthesized
gtgctagagtcgtaaacgacaagttagcattaggcttgtccgcaatgaacctgaaaaggtg

SEQ ID NO: 116 - DNA, 61 bp, Artificial Sequence
  Chemically Synthesized
cattttggcgtcgaaagacgaagtaaaatgaaggcgagaccgatatcaactggaagcagtg

SEQ ID NO: 117 - DNA, 61 bp, Artificial Sequence
  Chemically Synthesized
tttttagaggaaggaattccaagttaaaaaaaggcaggaccgggaacatgttgaaaaacag

SEQ ID NO: 118 - DNA, 61 bp, Artificial Sequence
  Chemically Synthesized
cttaccgaactaggaatagtaagtggtaagaaggcctgaccgtaataagcctgaaaaggcg

SEQ ID NO: 119 - DNA, 4186 bp, Artificial Sequence
  Chemically Synthesized, ELSA Succinate
agttacacttaccctactttatcggattctgaggaacaggagactgattattgacattagcacttgagctgattataatgggccgctctttcgttaccgccgattgttctagagctggtaacagcaagttagaataagtctagtccattatcaactggaaacagtggccagaaagggtcctgaatttcagggccctttttttacatttcacgccgattgtccttcaggttttacagaataagataactacggatagttgacatgcgtgatttaacattctataattgcacataaggttaagacgcttaacggatgtagatgtagaaatacaaggttacattaaggcccgtccgtaatcaacttgaagaagtgttccatcgggtccgaattttcggaccttttctccgcatactgataatagttgattgtctgaagtgtaaaccctccaccgaaaggcttttgacagaccttatctacatggttataatctgaataccatttactgcatcgatgagttgtagatctagaaatagaatgttacaattaggctagtccgttatgaacatgaaaatgtgagaaaagaggccgcgaaagcggccttttttcgtttatactgagagcgttgaccaataaggattacactatcttctgttgtgacacttgacacggatcttcgctgaacgtataatgagaaagaaaccctgctgttgcatcggttttagagtgagaaatcacaagttaaaataaggctagaccgttatcaactagaaatagtgttattgaacacccaaatcgggtgtttttttgtttgattacactgtgatacttctgacgcaggttttccaacgagttacgaataattgacaggtgaacgctcagctcttataatgcctatagccgtttcctgccggagtacttttagagatagaaatatcaagttaaaagaaggctagtccgttaccaacttgaaaaagtgagaaaaaaggcacgtcatctgacgtgccttttttatttaagtgaggtgactacttctctgaaatcgtttacaacccttcggaataagatttgacaactgctcagcgaaatactataatgactactttatcctgaacagtgatccgctttagagctagaaatagcaggttaaagtaaggccagtccgtaataaactggaaacagtgagaaaagagacgctttcgagcgtcttttttcgtttgaaccagtctcgtagttgttacagcgataagaataggtgttgaaatactcttgacagcatctgctttgtcacctataattcaatgagctgcaaccgtttgtttcagttttggacctagaaataggaagtcaaaataaggctggaccgacatgtaatcgaaagatttagtcaaaagcctccggtcggaggcttttgactttctgtaagacagagtagggtattatcactattcgctggaacttcaccaatctttgacacctcatcttatagttcctataatttctatcggagacctggcggcagtatgttggagagcaagacattgcaagttccaataaggcgtgtccgataaaagcttgagaaagcaaagtaatacaaaacaggcccaggcggcctgttttgtctttttaatgtaataatcccagactcagaataggaatcttacctgtcgtgttgcgaagttttgacataagtcgtattcaaagatataatataggtagccaaaatccatcatcatgatctgagagccaaaaatggcaagttcagataaggccagaccgttaccagcttaaataagcgatcctaaagccccgaattttttataaattcggggcttttttactagtagtttattcgctctattgaggtagtcgtcagaacccttatcaggaaaagttgacatcctctcgtaggactcatataatactcagagtctcttttttctgtatcgtttttagaggaaggaattccaagttaaaaaaaggcaggaccgggaacatgttgaaaaacaggcaaaaaagcgcctttagggcgcttttttacattagatagtgcgaagtcttatttggatccctacacgagagtgatttcaaccgtattgacaccgggttgaatactatctataatgtacggtacgtggctaaaaaaacgtcggaatagaaaacaaaagtttaagttattctaaggccagtccggaatcatcctaaaaaggagttattgaacacccgaaagggtgtttttttgtttaggcagttatctcttaccgagttttacttcagtgtgcgaatagacaacaattgacatttcgtcaagagtcgactataatatcgcggtctgtaggtccagattaacgatttcgagctaggcatagcaagtgaaattaaggctggtccattaacaccttgaaaaagggaacaataaggcctccctttagggggggccttttttattgaaaagttcaccgttattatcctgtaggtagtattttcagccaccagagtagttgacagtcctcgaacacctctatataatagtgtcgaagaagctgcccataatcgttttcagatttggaaacaaaacgttgaaaaaaggcaagtccgttatgaacgcgaaagcgtgcgaaaaaacccgcttcggcgggtttttttatagtttccagaggaccttcacggataaaatagattacagttctcgtcgtagtattgacatgagctcgtcgtcaggatatatagcttttgctaaaggatacctgataggttgtagagctagcaatagcaggttacaataaggctcgtccgttataaacatgaaaatgtgactaaaaaggccgctctgcggccttttttcttttttgattgaaggaccgtagcagtcacagagtgtaactttattcccagtaatttgacacattaggatggacgtattataatatgcccagacagttcacgaaccattggttttagatcacgaaagtgaaagttaaaataagcctagcccgttaccaactggaaacagtgacttaagaccgccggtcttgtccactaccttgcagtaatgcggtggacaggatcggcggttttcttttcttccgtagataatagaataaggtgccctcagattgttggaagcgacttttattgacactatggtccgcaagcattataatgctctgaacaagtttagttcatctgagtatctgaactcgacagagtaagtagatataaggccagtccgttagcaacttgaaaaagtcggagataaaaccgaccacggcaccaggcagtgaccatgtggtttcttcatcctctcacttctctgttggcacgaaaagggcaataagatttacggattactatcttgacatgaagtgttagacgtcatataatcgtggtgcgggtggacatccactcgagcttcagatccagaaatggaaagttgaagtgaggcaggtccggtagcaactcgaaagagtgagaaaagaggggagcgggaaaccgctccccttttttcgtttctgaataagcactgttgataatcgcaatctgtctcttcgtgaaaagtagcttgacaatcgctgtctacgtgaatataatgaatttcaatggcagtgtggcactcacattttggcgtcgaaagacgaagtaaaatgaaggcgagaccgatatcaactggaagcagtgtctggtagtcctggtaagacgcgaacagcgtcgcatcaggcatattgccaactagagactactattgtcgtttattatcgcaacagagggaagttcactgacctattgacatgactctccagctgtgctataattgtactatttgccgaaacgtgcagccgtttaagagctagaaatagcacgtttaaataaggctagtccgttttcaacttgaaaaagtgataacaaagccgggtaattcccggctttgttgtatcgtgaacgacactactatttcttacgagatacttattctggaagcaacggtttgacataggcaagccagtatagtataatcacatacaccgcaccagcgactggaccttaccgaactaggaatagtaagtggtaagaaggcctgaccgtaataagcctgaaaaggcgaccaaaaaggggggattttatctcccctttaatttttcagactccctgtatcgttgaaaagttggaacactgtgaatcctattactga

SEQ ID NO: 120 - DNA, 676 bp, Artificial Sequence
  Chemically Synthesized, ELSA MultiAux
ataggatcctagtttattcgctctattgaggtagtcgtcagaacccttatcttgacatttcgtcaagagtcgactataatatcgcggcgatagttgatcctcagcggttttagatcacgaaagtgaaagttaaaataagcctagcccgttaccaactggaagcagtgtctggtagtcctggtaagacgcgaacagcgtcgcatcaggcatattgccaactagctgaataagcactgttgataatcgcaatctgtctcttcgtgaaaagtagcttgacacggatcttcgctgaacgtataatgagaaatactgtactaaagtcacttagttttggacctagaaataggaagtcaaaataaggctggaccgacatgtaatcgaaagatttagtcaaaagcctccggtcggaggcttttgactttcgtgaacgacactactatttcttacgagatacttattctggaagcaacggtttgacacagcccagccggagagtataatcctattattaaacgcatcataaaaatcttaccgaactaggaatagtaagtggtaagaaggcctgaccgtaataagcctgaaaaggcgaccaaaaaggggggattttatctcccctttaatttttcaaaggtggtatttattacgcagacaactccctgagaacggttttcaatct

SEQ ID NO: 121 - DNA, 2551 bp, Artificial Sequence
  Chemically Synthesized, ELSA MultiAux
ataggatcctagtttattcgctctattgaggtagtcgtcagaacccttatcttgacatttcgtcaagagtcgactataatatcgcgattttttttgatattgatttgttttagatcacgaaagtgaaagttaaaataagcctagcccgttaccaactggaaacagtgacttaagaccgccggtcttgtccactaccttgcagtaatgcggtggacaggatcggcggttttcttttctgaaccagtctcgtagttgttacagcgataagaataggtgttgaaatactcttgacatgagctcgtcgtcaggatatatagctttgtgggtgccagcggctacgcgttgtagagctagcaatagcaggttacaataaggctcgtccgttataaacatgaaaatgtgttcacaaatgccgccactcaaacagagcggcatttttcttccccatctcttaccgagttttacttcagtgtgcgaatagacaacaattgacagaggcagtactaccgtttataattcggacagagttactggatacaaaaaggaatagaaaacaaaagtttaagttattctaaggccagtccggaatcatcctaaaaaggagttattgaacacccgaaagggtgtttttttgttttgtgagacttatttatcccgaaactattgtgttactgaagcaaccgcagattgacatgcgtgatttaacattctataattgcacaatttttgcatctaatcaacggatttcgagctaggcatagcaagtgaaattaaggctggtccattaacaccttgaaaaagggaacaataaggcctccctttagggggggccttttttattgatgaaaagcaatccctcgtgaagtaactcaatagtgttctctggtatcgtattgacataagtcgtattcaaagatataatataggtactttcacgttagaaagcaattttcagatttggaaacaaaacgttgaaaaaaggcaagtccgttatgaacgcgaaagcgtgcgaaaaaacccgcttcggcgggtttttttatagtttccagaggaccttcacggataaaatagattacagttctcgtcgtagtattgacagttgtgttatccggccatataatatctctcactttcacgttagaaagcagatgtagatgtagaaatacaaggttacattaaggcccgtccgtaatcaacttgaagaagtgttccatcgggtccgaattttcggaccttttctccgcattacaatcagcagtcagaacttttacgaagaatagtggtcgctcaaccttttgacagtgtgctaaaatttgtctataatgagtactcggtgctgaacagtgaatggttggagagcaagacattgcaagttccaataaggcgtgtccgataaaagcttgagaaagcaaagtaatacaaaacaggcccaggcggcctgttttgtctttttaatgtccgtagataatagaataaggtgccctcagattgttggaagcgacttttattgacagcatctgctttgtcacctataattcaatggcaaaaatgatatggattacatctgagagccaaaaatggcaagttcagataaggccagaccgttaccagcttaaataagcgatcctaaagccccgaattttttataaattcggggcttttttactagagtatcgtgaaaacctttattaccacactctgaactgtaggacgggatttttgacagaccttatctacatggttataatctgaatattgatactatcatgaccaggcttcagatccagaaatggaaagttgaagtgaggcaggtccggtagcaactcgaaagagtgagaaaagaggggagcgggaaaccgctccccttttttcgttttatcgtattcgtcacaccagattggcgtaagaagtcgctattgaaactatttgacactttgcacatgtcccgttataatcatgatacattcaccttacggctggtcattttggcgtcgaaagacgaagtaaaatgaaggcgagaccgatatcaactggaagcagtgtctggtagtcctggtaagacgcgaacagcgtcgcatcaggcatattgccaactagctgaataagcactgttgataatcgcaatctgtctcttcgtgaaaagtagcttgacacggatcttcgctgaacgtataatgagaaaataaacagaactatgccggagttttggacctagaaataggaagtcaaaataaggctggaccgacatgtaatcgaaagatttagtcaaaagcctccggtcggaggcttttgactttcgtgaacgacactactatttcttacgagatacttattctggaagcaacggtttgacacagcccagccggagagtataatcctattttcattgttttgataatcgccttaccgaactaggaatagtaagtggtaagaaggcctgaccgtaataagcctgaaaaggcgaccaaaaaggggggattttatctcccctttaatttttcaaaggtggtatttattacgcagacaactccctgagaacggttttcaatct

SEQ ID NO: 122 - DNA, 2551 bp, Artificial Sequence
  Chemically Synthesized, ELSA Stress
ataggatcctagtttattcgctctattgaggtagtcgtcagaacccttatcttgacatttcgtcaagagtcgactataatatcgcgttatgaaggaatcttcgttggttttagatcacgaaagtgaaagttaaaataagcctagcccgttaccaactggaaacagtgacttaagaccgccggtcttgtccactaccttgcagtaatgcggtggacaggatcggcggttttcttttctgaaccagtctcgtagttgttacagcgataagaataggtgttgaaatactcttgacatgagctcgtcgtcaggatatatagcttttgcaggggataatattgcccgttgtagagctagcaatagcaggttacaataaggctcgtccgttataaacatgaaaatgtgttcacaaatgccgccactcaaacagagcggcatttttcttccccatctcttaccgagttttacttcagtgtgcgaatagacaacaattgacagaggcagtactaccgtttataattcggacgcgtattctcgtatcagaccggaatagaaaacaaaagtttaagttattctaaggccagtccggaatcatcctaaaaaggagttattgaacacccgaaagggtgtttttttgttttgtgagacttatttatcccgaaactattgtgttactgaagcaaccgcagattgacatgcgtgatttaacattctataattgcacattgccgattcccctgtaagtgatttcgagctaggcatagcaagtgaaattaaggctggtccattaacaccttgaaaaagggaacaataaggcctccctttagggggggccttttttattgatgaaaagcaatccctcgtgaagtaactcaatagtgttctctggtatcgtattgacataagtcgtattcaaagatataatataggtgaaagtcatgggaaattctgttttcagatttggaaacaaaacgttgaaaaaaggcaagtccgttatgaacgcgaaagcgtgcgaaaaaacccgcttcggcgggtttttttatagtttccagaggaccttcacggataaaatagattacagttctcgtcgtagtattgacagttgtgttatccggccatataatatctctttaagtgtaggacagtacacgatgtagatgtagaaatacaaggttacattaaggcccgtccgtaatcaacttgaagaagtgttccatcgggtccgaattttcggaccttttctccgcattacaatcagcagtcagaacttttacgaagaatagtggtcgctcaaccttttgacagtgtgctaaaatttgtctataatgagtactaatctttaaaagattgtgagttggagagcaagacattgcaagttccaataaggcgtgtccgataaaagcttgagaaagcaaagtaatacaaaacaggcccaggcggcctgttttgtctttttaatgtccgtagataatagaataaggtgccctcagattgttggaagcgacttttattgacagcatctgctttgtcacctataattcaatggctacgcaaacccgaatcatatctgagagccaaaaatggcaagttcagataaggccagaccgttaccagcttaaataagcgatcctaaagccccgaattttttataaattcggggcttttttactagagtatcgtgaaaacctttattaccacactctgaactgtaggacgggatttttgacagaccttatctacatggttataatctgaatttgttattacccgacttacagcttcagatccagaaatggaaagttgaagtgaggcaggtccggtagcaactcgaaagagtgagaaaagaggggagcgggaaaccgctccccttttttcgttttatcgtattcgtcacaccagattggcgtaagaagtcgctattgaaactatttgacactttgcacatgtcccgttataatcatgatttctcgtatcagaccaggcacattttggcgtcgaaagacgaagtaaaatgaaggcgagaccgatatcaactggaagcagtgtctggtagtcctggtaagacgcgaacagcgtcgcatcaggcatattgccaactagctgaataagcactgttgataatcgcaatctgtctcttcgtgaaaagtagcttgacacggatcttcgctgaacgtataatgagaaactggcaagtaattagttgcagttttggacctagaaataggaagtcaaaataaggctggaccgacatgtaatcgaaagatttagtcaaaagcctccggtcggaggcttttgactttcgtgaacgacactactatttcttacgagatacttattctggaagcaacggtttgacacagcccagccggagagtataatcctattacgaagattccttcataacccttaccgaactaggaatagtaagtggtaagaaggcctgaccgtaataagcctgaaaaggcgaccaaaaaggggggattttatctcccctttaatttttcaaaggtggtatttattacgcagacaactccctgagaacggttttcaatct

SEQ ID NO: 123 - DNA, 2121 bp, Artificial Sequence
  Chemically Synthesized, ELSA Stress
ataggatccatatctcttagcggccgcagttttacttcagtgtgcgaatagacaacaattgacagaggcagtactaccgtttataattcggacagtctcttttttctgtatcgggaatagaaaacaaaagtttaagttattctaaggccagtccggaatcatcctaaaaaggagttattgaacacccgaaagggtgtttttttgttttgtgagacttatttatcccgaaactattgtgttactgaagcaaccgcagattgacatgcgtgatttaacattctataattgcacatggctttccaataatgcagggatttcgagctaggcatagcaagtgaaattaaggctggtccattaacaccttgaaaaagggaacaataaggcctccctttagggggggccttttttattgatgaaaagcaatccctcgtgaagtaactcaatagtgttctctggtatcgtattgacataagtcgtattcaaagatataatataggttgaacaatgtttattcatccttttcagatttggaaacaaaacgttgaaaaaaggcaagtccgttatgaacgcgaaagcgtgcgaaaaaacccgcttcggcgggtttttttataggagattactttacagaagactcacttatttcacggaactggtgctgacaattgacagttgtgttatccggccatataatatctctgcacccattcccgcgaaacggatgtagatgtagaaatacaaggttacattaaggcccgtccgtaatcaacttgaagaagtgttccatcgggtccgaattttcggaccttttctccgcattacaatcagcagtcagaacttttacgaagaatagtggtcgctcaaccttttgacagtgtgctaaaatttgtctataatgagtacatcgtcgacatattgctgccgttggagagcaagacattgcaagttccaataaggcgtgtccgataaaagcttgagaaagcaaagtaatacaaaacaggcccaggcggcctgttttgtctttttaatgtccgtagataatagaataaggtgccctcagattgttggaagcgacttttattgacagcatctgctttgtcacctataattcaatgaaaaatccataataaatataatctgagagccaaaaatggcaagttcagataaggccagaccgttaccagcttaaataagcgatcctaaagccccgaattttttataaattcggggcttttttactagagtatcgtgaaaacctttattaccacactctgaactgtaggacgggatttttgacagaccttatctacatggttataatctgaatgctcttgttataaatgggaagcttcagatccagaaatggaaagttgaagtgaggcaggtccggtagcaactcgaaagagtgagaaaagaggggagcgggaaaccgctccccttttttcgttttatcgtattcgtcacaccagattggcgtaagaagtcgctattgaaactatttgacactttgcacatgtcccgttataatcatgataaacagataagcctgaattacattttggcgtcgaaagacgaagtaaaatgaaggcgagaccgatatcaactggaagcagtgtctggtagtcctggtaagacgcgaacagcgtcgcatcaggcatattgccaactagctgaataagcactgttgataatcgcaatctgtctcttcgtgaaaagtagcttgacacggatcttcgctgaacgtataatgagaaactttttcattaaagcaatcagttttggacctagaaataggaagtcaaaataaggctggaccgacatgtaatcgaaagatttagtcaaaagcctccggtcggaggcttttgactttcgtgaacgacactactatttcttacgagatacttattctggaagcaacggtttgacacagcccagccggagagtataatcctattaatcattattttctcaaatgcttaccgaactaggaatagtaagtggtaagaaggcctgaccgtaataagcctgaaaaggcgaccaaaaaggggggattttatctcccctttaatttttcaaaggtggtatttattacgcagacaactccctgagaacggttttcaatct

SEQ ID NO: 124 - DNA, 20 bp, Artificial Sequence
  Chemically Synthesized
gattcgatcgattcgggaca

SEQ ID NO: 125 - DNA, 20 bp, Artificial Sequence
  Chemically Synthesized
ggagccccaggcgattatct

SEQ ID NO: 126 - DNA, 20 bp, Artificial Sequence
  Chemically Synthesized
aatcctctatctagggctta

SEQ ID NO: 127 - DNA, 20 bp, Artificial Sequence
  Chemically Synthesized
aggcgcagcgagatatctat

SEQ ID NO: 128 - DNA, 20 bp, Artificial Sequence
  Chemically Synthesized
attattgaatttccaaccag

SEQ ID NO: 129 - RNA, 61 nt, Artificial Sequence
  Chemically Synthesized
guuuuagagcuagaaauagcaaguuaaaauaaggcuaguccguuaucaacuugaaaaagug

SEQ ID NO: 130 - RNA, 61 nt, Artificial Sequence
  Chemically Synthesized
guuuuggagcuagaaauagcaagucaaaauaaggcuaguccguucucaacuugaaaaagug

SEQ ID NO: 131 - RNA, 61 nt, Artificial Sequence
  Chemically Synthesized
guuuuagaucacgaaagugaaaguuaaaauaagccuagcccguuaccaacuggaaacagug

SEQ ID NO: 132 - RNA, 61 nt, Artificial Sequence
  Chemically Synthesized
gguuuagaguuagaaauaacaaguuaaacuaaggcuaguccguuauaaacuugaaaaaguc

SEQ ID NO: 133 - RNA, 61 nt, Artificial Sequence
  Chemically Synthesized
guuguagaucuagaaauagaauguuacaauuaggcuaguccguuaugaacaugaaaaugug

SEQ ID NO: 134 - RNA, 61 nt, Artificial Sequence
  Chemically Synthesized
guuuuauagcuagaaauagcaagauaaaauaaggcuaguccauuaucaacuugaaaaagug

SEQ ID NO: 135 - RNA, 61 nt, Artificial Sequence
  Chemically Synthesized
guucuagagcugguaacagcaaguuagaauaagucuaguccauuaucaacuggaaacagug

SEQ ID NO: 136 - RNA, 61 nt, Artificial Sequence
  Chemically Synthesized
guuuuagagcuugaaaaagcaaguuaaaauuaggcuaguccguuaacaacuugaaaaagug

SEQ ID NO: 137 - RNA, 61 nt, Artificial Sequence
  Chemically Synthesized
guuuuugagcgagaaaucgcaaguaaaaauaaggcucguccguuaacaaguugaaaaacug

SEQ ID NO: 138 - RNA, 61 nt, Artificial Sequence
  Chemically Synthesized
guuuuagagauggaaacaucaaguuaaaauaaggcaaguccguuaacaacucgaaagagug

SEQ ID NO: 139 - RNA, 61 nt, Artificial Sequence
  Chemically Synthesized
guuugagagaucgaaagaucaaguucaaacaagucuaguccguugugaaccugaaaaggug

SEQ ID NO: 140 - RNA, 61 nt, Artificial Sequence
  Chemically Synthesized
guucuagagcucgaaagagcaacuuagaauaagccuaaucccugaucaacuugaaaaaguc

SEQ ID NO: 141 - RNA, 61 nt, Artificial Sequence
  Chemically Synthesized
guuuucgagcuaguaauagcaagugaaaaugaaguuaguccguuagcaaacugaaaaguua

SEQ ID NO: 142 - RNA, 61 nt, Artificial Sequence
  Chemically Synthesized
guguuagaguuggaaacaacaaguuaacauaaggcuacucggauuucaacgugaaaacguc

SEQ ID NO: 143 - RNA, 61 nt, Artificial Sequence
  Chemically Synthesized
guuuuagagcuagcaauagcaagcuaaaauaaugcuaguccguuauuaacuugaaaaagug

SEQ ID NO: 144 - RNA, 61 nt, Artificial Sequence
  Chemically Synthesized
guuuugcagcuagaaauagcaaggcaaaauaaugcuaguccguucccaacuugaaaaagug

SEQ ID NO: 145 - RNA, 61 nt, Artificial Sequence
  Chemically Synthesized
guuuuagagcuacacauagcaaguuaaaauaaagguaguccguuaucaguuugaaaaaacg

SEQ ID NO: 146 - RNA, 61 nt, Artificial Sequence
  Chemically Synthesized
guauuagagcuagaaauagcaacuuaauauaaggcuagucgguuaucaccuugaaaaaggg

SEQ ID NO: 147 - RNA, 61 nt, Artificial Sequence
  Chemically Synthesized
guaucugaacucgacagaguaaguagauauaaggccaguccguuagcaacuugaaaaaguc

SEQ ID NO: 148 - RNA, 61 nt, Artificial Sequence
  Chemically Synthesized
guuguagaggaagaaauuccaaguuacaaugaggcuaguccgugaugaacuugaaaaagug

SEQ ID NO: 149 - RNA, 61 nt, Artificial Sequence
  Chemically Synthesized
cuuuuagagauagaaauaucaaguuaaaagaaggcuaguccguuaccaacuugaaaaagug

SEQ ID NO: 150 - RNA, 61 nt, Artificial Sequence
  Chemically Synthesized
guuuaagagcuagaaauagcacguuuaaauaaggcuaguccguuuucaacuugaaaaagug

SEQ ID NO: 151 - RNA, 61 nt, Artificial Sequence
  Chemically Synthesized
guuuuagagugagaaaucacaaguuaaaauaaggcuagaccguuaucaacuagaaauagug

SEQ ID NO: 152 - RNA, 61 nt, Artificial Sequence
  Chemically Synthesized
gauuuagagcuggaaacagcaaguuaaauuaaggcuaguccguuaucagcuugaaaaagcg

SEQ ID NO: 153 - RNA, 61 nt, Artificial Sequence
  Chemically Synthesized
gcuuuagaccuugaaaaaggaaguuaaaguaaggcuaguccguuaugaccuugaaaaaggg

SEQ ID NO: 154 - RNA, 61 nt, Artificial Sequence
  Chemically Synthesized
guuuuagaccuagaaauaggaaguuaaaauaaggcuaguucguuaucaucuugaaaaagag

SEQ ID NO: 155 - RNA, 61 nt, Artificial Sequence
  Chemically Synthesized
gcuuuagagcuagaaauagcagguuaaaguaaggccaguccguaauaaacuggaaacagug

SEQ ID NO: 156 - RNA, 61 nt, Artificial Sequence
  Chemically Synthesized
gauuucgagcuaggcauagcaagugaaauuaaggcugguccauuaacaccuugaaaaaggg

SEQ ID NO: 157 - RNA, 61 nt, Artificial Sequence
  Chemically Synthesized
guuguagagcuagcaauagcagguuacaauaaggcucguccguuauaaacaugaaaaugug

SEQ ID NO: 158 - RNA, 61 nt, Artificial Sequence
  Chemically Synthesized
guuuaaggguuagaaauaacaaguuuaaauaaggcaaguccguuaucaaguggcaacacuc

SEQ ID NO: 159 - RNA, 61 nt, Artificial Sequence
  Chemically Synthesized
guuguagagcuagaaauagcgaguuacaauaaggcuaguccguuaugaacuugaaaaagug

SEQ ID NO: 160 - RNA, 61 nt, Artificial Sequence
  Chemically Synthesized
gcuuuauaucuagaaauagagagauaaaguaaggcaaguccguuaucaucuggaaacagac

SEQ ID NO: 161 - RNA, 61 nt, Artificial Sequence
  Chemically Synthesized
guuuuacaccuagaaauaggaagguaaaauaaggcugguccguuaucaccucgaaagaggg

SEQ ID NO: 162 - RNA, 61 nt, Artificial Sequence
  Chemically Synthesized
gcuuuacagcuagaaauagcaggguaaaguaaggcuaguccguaauaaacgugaaaacgug

SEQ ID NO: 163 - RNA, 61 nt, Artificial Sequence
  Chemically Synthesized
guuuuacagcuagugauagcaagguaaaauaaggcuagucccaaaucaacuugaaaaagug

SEQ ID NO: 164 - RNA, 61 nt, Artificial Sequence
  Chemically Synthesized
ggaauagaaaacaaaaguuuaaguuauucuaaggccaguccggaaucauccuaaaaaggag

SEQ ID NO: 165 - RNA, 61 nt, Artificial Sequence
  Chemically Synthesized
guuggagagcaagacauugcaaguuccaauaaggcguguccgauaaaagcuugagaaagca

SEQ ID NO: 166 - RNA, 61 nt, Artificial Sequence
  Chemically Synthesized
uuuuuagaggaaggaauuccaaguuaaaaaaaggcaggaccgggaacauguugaaaaacag

SEQ ID NO: 167 - RNA, 61 nt, Artificial Sequence
  Chemically Synthesized
cauuuuggcgucgaaagacgaaguaaaaugaaggcgagaccgauaucaacuggaagcagug

SEQ ID NO: 168 - RNA, 61 nt, Artificial Sequence
  Chemically Synthesized
gcuucagauccagaaauggaaaguugaagugaggcagguccgguagcaacucgaaagagug

SEQ ID NO: 169 - RNA, 61 nt, Artificial Sequence
  Chemically Synthesized
aucugagagccaaaaauggcaaguucagauaaggccagaccguuaccagcuuaaauaagcg

SEQ ID NO: 170 - RNA, 61 nt, Artificial Sequence
  Chemically Synthesized
gauguagauguagaaauacaagguuacauuaaggcccguccguaaucaacuugaagaagug

SEQ ID NO: 171 - RNA, 61 nt, Artificial Sequence
  Chemically Synthesized
uuuucagauuuggaaacaaaacguugaaaaaaggcaaguccguuaugaacgcgaaagcgug

SEQ ID NO: 172 - RNA, 61 nt, Artificial Sequence
  Chemically Synthesized
cuuaccgaacuaggaauaguaagugguaagaaggccugaccguaauaagccugaaaaggcg

SEQ ID NO: 173 - RNA, 61 nt, Artificial Sequence
  Chemically Synthesized
guuuuggaccuagaaauaggaagucaaaauaaggcuggaccgacauguaaucgaaagauuu

SEQ ID NO: 174 - RNA, 61 nt, Artificial Sequence
  Chemically Synthesized
aguuuagagaaugcaaauucaaguuaaacuaaggcgaguccgguauaaucguguaaacgag

SEQ ID NO: 175 - RNA, 61 nt, Artificial Sequence
  Chemically Synthesized
guaauagagauggauacaucaaguuauuauaaggcucgaccguuaacagucugaaaagacg

SEQ ID NO: 176 - RNA, 61 nt, Artificial Sequence
  Chemically Synthesized
guaugcgagguagaaauacccagugcauaucaggcuaguccgauaucauguugaagaacag

SEQ ID NO: 177 - RNA, 61 nt, Artificial Sequence
  Chemically Synthesized
gugcuagagucguaaacgacaaguuagcauuaggcuuguccgcaaugaaccugaaaaggug

SEQ ID NO: 178 - RNA, 61 nt, Artificial Sequence
  Chemically Synthesized
uuacuagagugacaaaucacaaguuaguaaaaggcuagaccguuauaaucccgaacgggag

SEQ ID NO: 179 - RNA, 61 nt, Artificial Sequence
  Chemically Synthesized
ccuuaagagcuagcaauagcaaguuuaaggaaggcaagcccguuaucauccugaauaggac

SEQ ID NO: 180 - RNA, 61 nt, Artificial Sequence
  Chemically Synthesized
cgguuaggauaagaaauuauaaguuaaccguaggcuagcccguuauaaacuggaaacagug

SEQ ID NO: 181 - RNA, 61 nt, Artificial Sequence
  Chemically Synthesized
guguuagagucagauaugacauguuaacauuaggcuaguccggggugaaguugaaaaacug

SEQ ID NO: 182 - DNA, 29 bp, Artificial Sequence
  Chemically Synthesized
cccgtttttttggataggaggatgaaacg

SEQ ID NO: 183 - DNA, 29 bp, Artificial Sequence
  Chemically Synthesized
cccgtttttttggataggaggaggtmkrg

SEQ ID NO: 184 - DNA, 29 bp, Artificial Sequence
  Chemically Synthesized
cccgtttttttggataggaggaggtatag

SEQ ID NO: 185 - DNA, 29 bp, Artificial Sequence
  Chemically Synthesized
cccgtttttttggataggaggaggtatgg

SEQ ID NO: 186 - DNA, 29 bp, Artificial Sequence
  Chemically Synthesized
cccgtttttttggataggaggaggtaggg

SEQ ID NO: 187 - DNA, 29 bp, Artificial Sequence
  Chemically Synthesized
cccgtttttttggataggaggaggtagag

SEQ ID NO: 188 - DNA, 29 bp, Artificial Sequence
  Chemically Synthesized
cccgtttttttggataggaggaggtcgag

SEQ ID NO: 189 - DNA, 29 bp, Artificial Sequence
  Chemically Synthesized
cccgtttttttggataggaggaggtcggg

SEQ ID NO: 190 - DNA, 29 bp, Artificial Sequence
  Chemically Synthesized
cccgtttttttggataggaggaggtctgg

SEQ ID NO: 191 - DNA, 29 bp, Artificial Sequence
  Chemically Synthesized
cccgtttttttggataggaggaggtctag

SEQ ID NO: 192 - DNA, 23 bp, Artificial Sequence
  Chemically Synthesized
gaaagtcatcccaaattctgcgg

SEQ ID NO: 193 - DNA, 23 bp, Artificial Sequence
  Chemically Synthesized
gaaagtcatgggagattctgcgg

SEQ ID NO: 194 - DNA, 23 bp, Artificial Sequence
  Chemically Synthesized
gaaagtcatgggtgattctgcgg

SEQ ID NO: 195 - DNA, 23 bp, Artificial Sequence
  Chemically Synthesized
gaaagtcatgggaaattcgtcgg

SEQ ID NO: 196 - DNA, 23 bp, Artificial Sequence
  Chemically Synthesized
gaaagtcatggtcaattcttcgg

SEQ ID NO: 197 - DNA, 23 bp, Artificial Sequence
  Chemically Synthesized
gaaagtcatgtgaagtgctgcgg

SEQ ID NO: 198 - DNA, 23 bp, Artificial Sequence
  Chemically Synthesized
gaaagtcatgagacatttcgcgg

SEQ ID NO: 199 - DNA, 23 bp, Artificial Sequence
  Chemically Synthesized
gaaagtcatgggaaacacatcgg

SEQ ID NO: 200 - DNA, 23 bp, Artificial Sequence
  Chemically Synthesized
gaaagtcatgtgaaagcattcgg

SEQ ID NO: 201 - DNA, 23 bp, Artificial Sequence
  Chemically Synthesized
gaaagtcattggaaattctgcgg

SEQ ID NO: 202 - DNA, 23 bp, Artificial Sequence
  Chemically Synthesized
gaaaatcattggaaattctgcgg

SEQ ID NO: 203 - DNA, 23 bp, Artificial Sequence
  Chemically Synthesized
gaattttatgggaaattctgcgg

SEQ ID NO: 204 - DNA, 23 bp, Artificial Sequence
  Chemically Synthesized
gcgagctatgggaaattctgcgg

SEQ ID NO: 205 - DNA, 23 bp, Artificial Sequence
  Chemically Synthesized
gaaagttatcggacattccgcgg

SEQ ID NO: 206 - DNA, 23 bp, Artificial Sequence
  Chemically Synthesized
gcaagtcaggggacattgcgcgg

SEQ ID NO: 207 - DNA, 23 bp, Artificial Sequence
  Chemically Synthesized
gccagacatggggaattcagcgg

SEQ ID NO: 208 - DNA, 23 bp, Artificial Sequence
  Chemically Synthesized
gaacgtgatggtaacccccgcgg

SEQ ID NO: 209 - DNA, 23 bp, Artificial Sequence
  Chemically Synthesized
caaagtcggggaaacctccgcgg

SEQ ID NO: 210 - DNA, 23 bp, Artificial Sequence
  Chemically Synthesized
aaaagtcgtgttaagtgatacgg

SEQ ID NO: 211 - DNA, 20 bp, Artificial Sequence
  Chemically Synthesized
ttgacgagcgccctgtaaat

SEQ ID NO: 212 - DNA, 20 bp, Artificial Sequence
  Chemically Synthesized
ttgacgagcgccctgtaaat

SEQ ID NO: 213 - DNA, 20 bp, Artificial Sequence
  Chemically Synthesized
acagttccgtaataaatatc

SEQ ID NO: 214 - DNA, 20 bp, Artificial Sequence
  Chemically Synthesized
tctcgccattaaaggaatca

SEQ ID NO: 215 - DNA, 20 bp, Artificial Sequence
  Chemically Synthesized
gttactaataaaatcaatca

SEQ ID NO: 216 - DNA, 20 bp, Artificial Sequence
  Chemically Synthesized
ccaaatcatcaatgcaatca

SEQ ID NO: 217 - DNA, 20 bp, Artificial Sequence
  Chemically Synthesized
ccacaaagcgatcttcgtag

SEQ ID NO: 218 - DNA, 20 bp, Artificial Sequence
  Chemically Synthesized
gtaacgacggtgaaattctg

SEQ ID NO: 219 - DNA, 20 bp, Artificial Sequence
  Chemically Synthesized
tggtaatttcttctgcatcg

SEQ ID NO: 220 - DNA, 20 bp, Artificial Sequence
  Chemically Synthesized
ccagaagctggtaccagacc

SEQ ID NO: 221 - DNA, 21 bp, Artificial Sequence
  Chemically Synthesized
ttaacttcagaagaaattctg

SEQ ID NO: 222 - DNA, 20 bp, Artificial Sequence
  Chemically Synthesized
gaagacaaaacattgctgcc

SEQ ID NO: 223 - DNA, 20 bp, Artificial Sequence
  Chemically Synthesized
atagacatcaatcagttgca

SEQ ID NO: 224 - DNA, 20 bp, Artificial Sequence
  Chemically Synthesized
ttcattggaaataatgccgg

SEQ ID NO: 225 - DNA, 20 bp, Artificial Sequence
  Chemically Synthesized
ttcattggaaataatgccgg

SEQ ID NO: 226 - DNA, 20 bp, Artificial Sequence
  Chemically Synthesized
accccagccgtattgctgct

SEQ ID NO: 227 - DNA, 20 bp, Artificial Sequence
  Chemically Synthesized
tcgtagcgcaataattcagg

SEQ ID NO: 228 - DNA, 20 bp, Artificial Sequence
  Chemically Synthesized
taaaaaaacaaataaataca

SEQ ID NO: 229 - DNA, 20 bp, Artificial Sequence
  Chemically Synthesized
tgcttttaatttcggtatcg

SEQ ID NO: 230 - DNA, 20 bp, Artificial Sequence
  Chemically Synthesized
taccatcgaaaatatcgccc

SEQ ID NO: 231 - DNA, 20 bp, Artificial Sequence
  Chemically Synthesized
ttaatgcggctgataattgc

SEQ ID NO: 232 - DNA, 20 bp, Artificial Sequence
  Chemically Synthesized
tcaacgatatctatgccgca

SEQ ID NO: 233 - DNA, 20 bp, Artificial Sequence
  Chemically Synthesized
ttaatgcggctgataattgc